(19) United States
(12) Patent Application Publication
(10) Pub. No.: US 2026/0285856 A1
(43) Pub. Date: Sep. 24, 2026

(54) A 1,5-DIHYDRO-4H-PYRROLO[3,2-C] PYRIDIN-4-ONE FOR USE IN THE TREATMENT OF CANCER

(71) Applicant: ANTARES THERAPEUTICS, INC., Boston, MA (US)

(72) Inventors: Benjamin C. MILGRAM, Weston, MA (US); Ryan D. WHITE, Somerville, MA (US); David ST. JEAN, Natick, MA (US); Angel GUZMAN-PEREZ, Belmont, MA (US)

(73) Assignee: ANTARES THERAPEUTICS, INC., Boston, MA (US)

(21) Appl. No.: 19/490,934

(22) PCT Filed: Jun. 6, 2024
(86) PCT No.: PCT/US2024/032793

§ 371 Date: Dec. 7, 2025

Related U.S. Application Data
(60) Provisional application No. 63/471,901, filed on Jun. 8, 2023.

(51) Int. Cl. C07D 471/04; A61K 31/444

(52) U.S. Cl. CPC C07D 471/04; A61K 31/444

(57) ABSTRACT

This present application discloses a chemical entity of formula (I), a pharmaceutically acceptable salt and/or hydrate and/or cocrystal and/or drug combination thereof that inhibits epidermal growth factor receptor (EGFR, ERBB 1) and/or Human epidermal growth factor receptor 2 (HER2, ERBB2). The chemical entity of formula (I) is useful, e.g., for treating a condition, disease or disorder in which increased (e.g., excessive) EGFR and/or HER2 activation contributes to the pathology and/or symptoms and/or progression of the condition, disease or disorder (e.g., cancer) in a subject (e.g., a human). This application also provides compositions containing the same as well as methods of using and making the same.

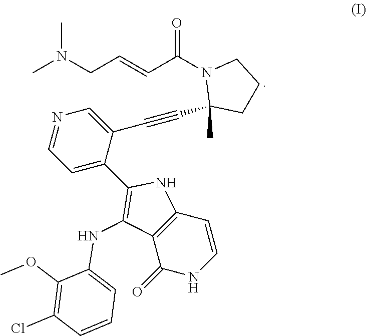 [SMILES: COc1c(Cl)cccc1Nc1c(-c2ccncc2C#C[C@@]2(C)CCCN2C(=O)/C=C/CN(C)C)[nH]c2cc[nH]c(=O)c12]

### CROSS-REFERENCE TO RELATED APPLICATIONS

This application claims the benefit of the filing date of U.S. Provisional Application No. 63/471,901, filed Jun. 8, 2023. The content of the application is incorporated by reference herein in its entirety.

### SEQUENCE LISTING

This application contains a Sequence Listing that has been submitted electronically as an XML file named “50006-0014W01_ST26_SL.XML.” The XML file, created on Jun. 4, 2024, is 5,182 bytes in size. The material in the XML file is hereby incorporated by reference in its entirety.

### TECHNICAL FIELD

This disclosure features chemical entities (e.g., a compound or a pharmaceutically acceptable salt, and/or hydrate, and/or cocrystal, and/or drug combination of the compound) that inhibits epidermal growth factor receptor (EGFR, ERBB1) and/or Human epidermal growth factor receptor 2 (HER2, ERBB2). The chemical entities are useful, e.g., for treating a condition, disease or disorder in which increased (e.g., excessive) EGFR and/or HER2 activation contributes to the pathology and/or symptoms and/or progression of the condition, disease or disorder (e.g., cancer) in a subject (e.g., a human). This disclosure also provides compositions containing the same as well as methods of using and making the same.

### BACKGROUND

Epidermal growth factor receptor (EGFR, ERBB1) and Human epidermal growth factor receptor 2 (HER2, ERBB2) are members of a family of proteins which regulate cellular processes implicated in tumor growth, including proliferation and differentiation. Several investigators have demonstrated the role of EGFR and HER2 in development and cancer (Reviewed in Salomon, et al., Crit. Rev. Oncol. Hematol. (1995) 19:183-232, Klapper, et al., Adv. Cancer Res. (2000) 77, 25-79 and Hynes and Stern, Biochim. Biophys. Acta (1994) 1198:165-184). EGFR overexpression is present in at least 70% of human cancers, such as non-small cell lung carcinoma (NSCLC), breast cancer, glioma, and prostate cancer. HER2 overexpression occurs in approximately 30% of all breast cancer. It has also been implicated in other human cancers including colon, ovary, bladder, stomach, esophagus, lung, uterus and prostate. HER2 overexpression has also been correlated with poor prognosis in human cancer, including metastasis, and early relapse.

EGFR and HER2 are, therefore, widely recognized as targets for the design and development of therapies that can specifically bind and inhibit tyrosine kinase activity and its signal transduction pathway in cancer cells, and thus can serve as diagnostic or therapeutic agents. For example, EGFR tyrosine kinase inhibitors (TKIs) are effective clinical therapies for EGFR mutant advanced non-small cell lung cancer (NSCLC) patients. However, the vast majority of patients develop disease progression following successful treatment with an EGFR TKI. Common mechanisms of resistance include acquired, secondary mutation T790M, C797S, and EGFR exon 20 insertion mutations. For example, NSCLC tumors can have EGFR exon 20 insertion mutations that are intrinsically resistant to current EGFR TKIs.

Overexpression of another protein, BUB1 (Budding uninhibited by benzimidazole, BUB1) kinase, is often associated with proliferating cells, including cancer cells, and tissues (Bolanos-Garcia VM and Blundell T L, Trends Biochem. Sci. 36, 141, 2010). This protein is an essential part of the complex network of proteins that form the mitotic checkpoint. The major function of an unsatisfied mitotic checkpoint is to keep the anaphase-promoting complex/cyclosome (APC/C) in an inactive state. As soon as the checkpoint gets satisfied the APC/C ubiquitin-ligase targets cyclin B and securin for proteolytic degradation leading to separation of the paired chromosomes and exit from mitosis.

Incomplete mitotic checkpoint function has been linked with aneuploidy and tumourigenesis (see Weaver B A and Cleveland D W, Cancer Res. 67, 10103, 2007; King R W, Biochim Biophys Acta 1786, 4, 2008). In contrast, complete inhibition of the mitotic checkpoint has been recognized to result in severe chromosome missegregation and induction of apoptosis in tumour cells (see Kops G J et al., Nature Rev. Cancer 5, 773, 2005; Schmidt M and Medema R H, Cell Cycle 5, 159, 2006; Schmidt M and Bastians H, Drug Res. Updates 10, 162, 2007). Thus, mitotic checkpoint inhibition through inhibition of BUB1 kinase represents an approach for the treatment of proliferative disorders, including solid tumors such as carcinomas, sarcomas, leukemias and lymphoid malignancies or other disorders, associated with uncontrolled cellular proliferation.

WO 2022/066734, which is incorporated herein by reference in its entirety, discloses compounds having the following formula:

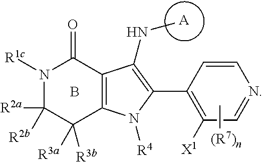

Said compounds are described as having activity as inhibitors of EGFR and/or HER2.

### SUMMARY

This disclosure features chemical entities (e.g., a compound or a pharmaceutically acceptable salt, and/or hydrate, and/or cocrystal, and/or drug combination of the compound) that inhibits epidermal growth factor receptor (EGFR, ERBB1) and/or Human epidermal growth factor receptor 2 (HER2, ERBB2). The chemical entities are useful, e.g., for treating a condition, disease or disorder in which increased (e.g., excessive) EGFR and/or HER2 activation contributes to the pathology and/or symptoms and/or progression of the condition, disease or disorder (e.g., cancer) in a subject (e.g., a human). This disclosure also provides compositions containing the same as well as methods of using and making the same.

In one aspect, the disclosure provides a compound of Formula (I):

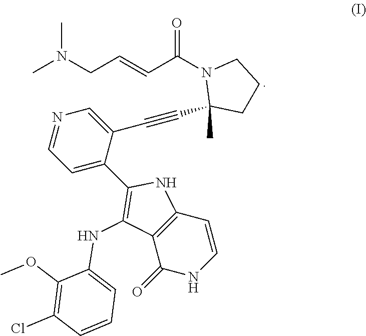 [SMILES: COc1c(Cl)cccc1Nc1c(-c2ccncc2C#C[C@@]2(C)CCCN2C(=O)/C=C/CN(C)C)[nH]c2cc[nH]c(=O)c12]

or a pharmaceutically acceptable salt or solvate thereof.

In another aspect, the disclosure provides a compound of Formula (I):

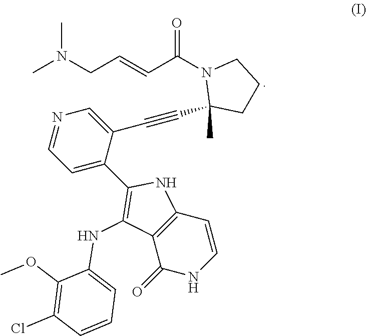 [SMILES: COc1c(Cl)cccc1Nc1c(-c2ccncc2C#C[C@@]2(C)CCCN2C(=O)/C=C/CN(C)C)[nH]c2cc[nH]c(=O)c12]

or a pharmaceutically acceptable salt thereof.

This disclosure is based, in part, on the finding that the compound of Formula (I) is generated, e.g., by oxidation of compound 362 in WO 2022/066734 or a stereoisomeric mixture thereof:

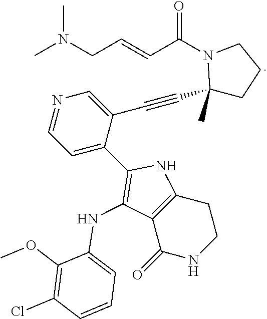 [SMILES: COc1c(Cl)cccc1Nc1c(-c2ccncc2C#C[C@@]2(C)CCCN2C(=O)/C=C/CN(C)C)[nH]c2c1C(=O)NCC2]

Compound 362 in WO 2022/066734 exhibits potent and selective inhibition of EGFR, e.g., exhibiting greater inhibition of EGFR containing one or more mutations relative to inhibition of wild type EGFR. For example, Compound 362 in WO 2022/066734 exhibits greater inhibition of EGFR containing an EGFR kinase protein insertion, e.g., an exon 20 insertion, relative to inhibition of wild type EGFR. In some embodiments, the exon 20 insertion is selected from the group consisting of: V769_D770insX, D770_N771insX, N771_P772insX, P772_H773insX, and H773_V774insX. For example, the exon 20 insertion is selected from the group consisting of: A767_V769dupASV, V769_D770insASV, D770_N771insNPG, D770_N771insNPY, D770_N771insSVD, D770_N771insGL, N771_H773dupNPH, N771_P772insN, N771_P772insH, N771_P772insV, P772_H773insDNP, P772_H773insPNP, H773_V774insNPH, H773_V774insH, H773_V774insPH, H773_V774insAH, and P772_H773insPNP; or any combination thereof; e.g., any two or more independently selected exon 20 insertions; e.g., any two independently selected exon 20 insertions (e.g., V769_D770insASV and D770_N771insSVD).

Compound 362 in WO 2022/066734 is useful, e.g., for the treatment of a cancer (e.g., lung cancer, e.g., non-small cell lung cancer) exhibiting any of the exon 20 insertions described above, e.g., V769_D770insASV or D770_N771insSVD.

In certain embodiments, the cancer is lung cancer, and the exon 20 insertion is V769_D770insASV.

In certain embodiments, the cancer is lung cancer, and the exon 20 insertion is D770 N771insSVD.

In certain embodiments, the cancer is non-small cell lung cancer, and the exon 20 insertion is V769_D770insASV.

In certain embodiments, the cancer is non-small cell lung cancer, and the exon 20 insertion is D770_N771insSVD.

The compound of Formula (I) can be generated, e.g., in vivo, e.g., from compound 362 in WO 2022/066734 or a stereoisomeric mixture thereof. For example, the compound described herein can be generated, e.g., in vivo in a mammal (e.g., a human) from compound 362 in WO 2022/066734 or a stereoisomeric mixture thereof.

The compound of Formula (I) also exhibits potent and selective inhibition of EGFR, e.g., exhibiting greater inhibition of EGFR containing one or more mutations relative to inhibition of wild type EGFR. For example, the compound of Formula (I) exhibits greater inhibition of EGFR containing an EGFR kinase protein insertion, e.g., an exon 20 insertion, relative to inhibition of wild type EGFR. In some embodiments, the exon 20 insertion is selected from the group consisting of: V769_D770insX, D770_N771insX, N771_P772insX, P772_H773insX, and H773_V774insX. For example, the exon 20 insertion selected from the group consisting of: A767_V769dupASV, V769_D770insASV, D770_N771insNPG, D770_N771insNPY, D770_N771insSVD, D770_N771insGL, N771_H773dupNPH, N771_P772insN, N771_P772insH, N771_P772insV, P772_H773insDNP, P772_H773insPNP, H773_V774insNPH, H773_V774insH, H773_V774insPH, H773_V774insAH, and P772_H773insPNP; or any combination thereof; e.g., any two or more independently selected exon 20 insertions; e.g., any two independently selected exon 20 insertions (e.g., V769_D770insASV and D770_N771insSVD).

Accordingly, the compound of Formula (I) is useful, e.g., for the treatment of a cancer (e.g., lung cancer, e.g., non-small cell lung cancer) exhibiting any one of the exon 20 insertions described above, e.g., V769_D770insASV and D770_N771insSVD. In certain embodiments, the cancer is lung cancer, and the exon 20 insertion is V769_D770insASV.

In certain embodiments, the cancer is lung cancer, and the exon 20 insertion is D770 N771insSVD.

In certain embodiments, the cancer is non-small cell lung cancer, and the exon 20 insertion is V769_D770insASV.

In certain embodiments, the cancer is non-small cell lung cancer, and the exon 20 insertion is D770_N771insSVD.

Advantageously, the compound of Formula (I) can also differentiate in various drug-like properties including, but not limited to, in vitro metabolic stability in human and/or pre-clinical species, membrane permeability, transporter active efflux, aqueous solubility, cytochrome P450 inhibition, cytochrome P450 induction, pharmacokinetics in pre-clinical species, binding kinetics, HERG inhibition, in vivo pharmacodynamics and/or efficacy, kinome selectivity, genetic toxicology, metabolite profile, and/or plasma and/or in vitro protein binding.

Further, and surprisingly, the compound of Formula (I) exhibits EGFR mutant potency and selectivity against EGFR WT that is greater than the potency and selectivity exhibited by its enantiomer (compound 635 in WO 2022/066734).

Also provided herein is a pharmaceutical composition comprising a compound of Formula (I), or a pharmaceutically acceptable salt thereof, and a pharmaceutically acceptable carrier.

Provided herein is a method for treating cancer in a subject in need thereof, the method comprising administering to the subject a therapeutically effective amount of a compound of Formula (I), or a pharmaceutically acceptable salt thereof, or a pharmaceutical composition as provided herein.

Also provided herein is a method for treating cancer in a subject in need thereof, the method comprising (a) determining that the cancer is associated with a dysregulation of an EGFR gene, an EGFR kinase, or expression or activity or level of any of the same; and (b) administering to the subject a therapeutically effective amount of a compound of Formula (I), or a pharmaceutically acceptable salt thereof, or a pharmaceutical composition as provided herein.

Provided herein is a method of treating an EGFR-associated disease or disorder in a subject, the method comprising administering to a subject identified or diagnosed as having an EGFR-associated disease or disorder a therapeutically effective amount of a compound of Formula (I), or a pharmaceutically acceptable salt thereof, or a pharmaceutical composition as provided herein.

This disclosure also provides a method of treating an EGFR-associated disease or disorder in a subject, the method comprising: determining that the cancer in the subject is an EGFR-associated disease or disorder; and administering to the subject a therapeutically effective amount of a compound of Formula (I), or a pharmaceutically acceptable salt thereof, or a pharmaceutical composition as provided herein.

Further provided herein is a method of treating an EGFR-associated cancer in a subject, the method comprising administering to a subject identified or diagnosed as having an EGFR-associated cancer a therapeutically effective amount of a compound of Formula (I), or a pharmaceutically acceptable salt thereof, or a pharmaceutical composition as provided herein.

This disclosure also provides a method of treating an EGFR-associated cancer in a subject, the method comprising: determining that the cancer in the subject is an EGFR-associated cancer; and administering to the subject a therapeutically effective amount of a compound of Formula (I), or a pharmaceutically acceptable salt thereof, or a pharmaceutical composition as provided herein.

Provided herein is a method of treating a subject, the method comprising administering a therapeutically effective amount of a compound of Formula (I), or a pharmaceutically acceptable salt thereof, or a pharmaceutical composition as provided herein, to a subject having a clinical record that indicates that the subject has a dysregulation of an EGFR gene, an EGFR kinase, or expression or activity or level of any of the same.

Also provided herein is a method of treating a subject having a cancer, wherein the method includes:
(a) administering one or more doses of a first EGFR inhibitor to the subject for a period of time;
(b) after (a), determining whether a cancer cell in a sample obtained from the subject has at least one EGFR inhibitor resistance mutation that confers increased resistance to a cancer cell or tumor to treatment with the first EGFR inhibitor of step (a); and
(c) administering a therapeutically effective amount of a compound of Formula (I), or a pharmaceutically acceptable salt thereof, as a monotherapy or in conjunction with another anticancer agent to the subject if the subject has been determined to have a cancer cell that has at least one EGFR inhibitor resistance mutation that confers increased resistance to a cancer cell or tumor to treatment with the first EGFR inhibitor of step (a); or
(d) administering additional doses of the first EGFR inhibitor of step (a) to the subject if the subject has not been determined to have a cancer cell that has at least one EGFR inhibitor resistance mutation that confers increased resistance to a cancer cell or tumor to treatment with the first EGFR inhibitor of step (a).

Further provided herein is a method of treating a subject having a cancer, wherein the method includes:
(a) determining whether a cancer cell in a sample obtained from a subject having a cancer and previously administered one or more doses of a first EGFR inhibitor has one or more EGFR inhibitor resistance mutations that confer increased resistance to a cancer cell or tumor to treatment with the first EGFR inhibitor that was previously administered to the subject; and
(b) administering a therapeutically effective amount of a compound of Formula (I), or a pharmaceutically acceptable salt thereof, as a monotherapy or in conjunction with another anticancer agent to the subject if the subject has been determined to have a cancer cell that has at least one EGFR inhibitor resistance mutation that confers increased resistance to a cancer cell or tumor to treatment with the first EGFR inhibitor that was previously administered to the subject; or
(c) administering additional doses of the first EGFR inhibitor to the subject if the subject has not been determined to have a cancer cell that has at least one EGFR inhibitor resistance mutation that confers increased resistance to a cancer cell or tumor to treatment with the first EGFR inhibitor previously administered to the subject.

Also provided herein is a method of treating a subject having a cancer, wherein the method comprises:
(a) determining that a cancer cell in a sample obtained from a subject having a cancer and previously administered one or more doses of a first EGFR inhibitor has one or more EGFR inhibitor resistance mutations that confer increased resistance to a cancer cell or tumor to treatment with the first EGFR inhibitor that was previously administered to the subject; and
(b) administering a therapeutically effective amount of a compound of Formula (I), or a pharmaceutically acceptable salt thereof, as a monotherapy or in conjunction with another anticancer agent to the subject.

Further provided herein is a method of treating a subject having a cancer, wherein the method comprises:
(a) determining that a cancer cell in a sample obtained from a subject having a cancer and previously administered one or more doses of a first EGFR inhibitor does not have one or more EGFR inhibitor resistance mutations that confer increased resistance to a cancer cell or tumor to treatment with the first EGFR inhibitor that was previously administered to the subject; and
(b) administering additional doses of the first EGFR inhibitor to the subject.

This disclosure also provides a method for inhibiting EGFR in a mammalian cell, the method comprising contacting the mammalian cell with an effective amount of a compound of Formula (I), or a pharmaceutically acceptable salt thereof.

Also provided herein is a method for treating cancer in a subject in need thereof, the method comprising (a) determining that the cancer is associated with a dysregulation of a HER2 gene, a HER2 kinase, or expression or activity or level of any of the same; and (b) administering to the subject a therapeutically effective amount of a compound of Formula (I), or a pharmaceutically acceptable salt thereof, or a pharmaceutical composition as provided herein.

Further provided herein is a method of treating a HER2-associated cancer in a subject, the method comprising administering to a subject identified or diagnosed as having a HER2-associated cancer a therapeutically effective amount of a compound of Formula (I), or a pharmaceutically acceptable salt thereof, or a pharmaceutical composition as provided herein.

This disclosure also provides a method of treating a HER2-associated cancer in a subject, the method comprising: determining that the cancer in the subject is a HER2-associated cancer; and administering to the subject a therapeutically effective amount of a compound of Formula (I), or a pharmaceutically acceptable salt thereof, or a pharmaceutical composition as provided herein.

Provided herein is a method of treating a subject having a cancer, the method comprising administering a therapeutically effective amount of a compound of Formula (I), or a pharmaceutically acceptable salt thereof, or a pharmaceutical composition as provided herein, to a subject having a clinical record that indicates that the subject has a dysregulation of a HER2 gene, a HER2 kinase, or expression or activity or level of any of the same.

Also provided herein is a method of treating a subject having a cancer, wherein the method comprises:
(a) administering one or more doses of a first HER2 inhibitor to the subject for a period of time;
(b) after (a), determining whether a cancer cell in a sample obtained from the subject has at least one HER2 inhibitor resistance mutation that confers increased resistance to a cancer cell or tumor to treatment with the first HER2 inhibitor of step (a); and
(c) administering a therapeutically effective amount of a compound of Formula (I), or a pharmaceutically acceptable salt thereof, as a monotherapy or in conjunction with another anticancer agent to the subject if the subject has been determined to have a cancer cell that has at least one HER2 inhibitor resistance mutation that confers increased resistance to a cancer cell or tumor to treatment with the first HER2 inhibitor of step (a); or
(d) administering additional doses of the first HER2 inhibitor of step (a) to the subject if the subject has not been determined to have a cancer cell that has at least one HER2 inhibitor resistance mutation that confers increased resistance to a cancer cell or tumor to treatment with the first HER2 inhibitor of step (a).

Further provided herein is a method of treating a subject having a cancer, wherein the method comprises:
(a) determining whether a cancer cell in a sample obtained from a subject having a cancer and previously administered one or more doses of a first HER2 inhibitor has one or more HER2 inhibitor resistance mutations that confer increased resistance to a cancer cell or tumor to treatment with the first HER2 inhibitor that was previously administered to the subject; and
(b) administering a therapeutically effective amount of a compound of Formula (I), or a pharmaceutically acceptable salt thereof, as a monotherapy or in conjunction with another anticancer agent to the subject if the subject has been determined to have a cancer cell that has at least one HER2 inhibitor resistance mutation that confers increased resistance to a cancer cell or tumor to treatment with the first HER2 inhibitor that was previously administered to the subject; or
(c) administering additional doses of the first HER2 inhibitor to the subject if the subject has not been determined to have a cancer cell that has at least one HER2 inhibitor resistance mutation that confers increased resistance to a cancer cell or tumor to treatment with the first HER2 inhibitor previously administered to the subject.

Also provided herein is a method of treating a subject having a cancer, wherein the method comprises:
(a) determining that a cancer cell in a sample obtained from a subject having a cancer and previously administered one or more doses of a first HER2 inhibitor has one or more HER2 inhibitor resistance mutations that confer increased resistance to a cancer cell or tumor to treatment with the first HER2 inhibitor that was previously administered to the subject; and
(b) administering a therapeutically effective amount of a compound of Formula (I), or a pharmaceutically acceptable salt thereof, as a monotherapy or in conjunction with another anticancer agent to the subject.

Further provided herein is a method of treating a subject having a cancer, wherein the method comprises:
(a) determining that a cancer cell in a sample obtained from a subject having a cancer and previously administered one or more doses of a first HER2 inhibitor does not have one or more HER2 inhibitor resistance mutations that confer increased resistance to a cancer cell or tumor to treatment with the first HER2 inhibitor that was previously administered to the subject; and
(b) administering additional doses of the first HER2 inhibitor to the subject.

This disclosure also provides a method for inhibiting HER2 in a mammalian cell, the method comprising contacting the mammalian cell with an effective amount of a compound of Formula (I), or a pharmaceutically acceptable salt thereof.

Also provided herein is a method for treating cancer in a subject in need thereof, the method comprising (a) determining that the cancer is associated with a dysregulation of an EGFR gene, an EGFR kinase, or expression or activity or level of any of the same and that the cancer is associated with a dysregulation of a HER2 gene, a HER2 kinase, or expression or activity or level of any of the same; and (b) administering to the subject a therapeutically effective amount of a compound of Formula (I), or a pharmaceutically acceptable salt thereof, or a pharmaceutical composition as provided herein.

Further provided herein is a method of treating an EGFR-associated and HER2-associated cancer in a subject, the method comprising administering to a subject identified or diagnosed as having an EGFR-associated and a HER2-associated cancer a therapeutically effective amount of a compound of Formula (I), or a pharmaceutically acceptable salt thereof, or a pharmaceutical composition as provided herein.

This disclosure also provides a method of treating an EGFR-associated and HER2-associated cancer in a subject, the method comprising: determining that the cancer in the subject is an EGFR-associated and a HER2-associated cancer; and administering to the subject a therapeutically effective amount of a compound of Formula (I), or a pharmaceutically acceptable salt thereof, or a pharmaceutical composition as provided herein.

Provided herein is a method of treating a subject, the method comprising administering a therapeutically effective amount of a compound of Formula (I), or a pharmaceutically acceptable salt thereof, or a pharmaceutical composition as provided herein, to a subject having a clinical record that indicates that the subject has a dysregulation of an EGFR gene, an EGFR kinase, or expression or activity or level of any of the same and a dysregulation of a HER2 gene, a HER2 kinase, or expression or activity or level of any of the same.

This disclosure also provides a method for inhibiting EGFR and HER2 in a mammalian cell, the method comprising contacting the mammalian cell with an effective amount of a compound of Formula (I), or a pharmaceutically acceptable salt thereof.

In addition to the above, provided herein is a method for inhibiting a BUB (budding uninhibited by benzimidazole, BUB1-3) kinase. In some embodiments, the methods provided herein include methods for inhibiting BUB11. For example, a method for inhibiting BUB1 in a mammalian cell, the method comprising contacting the mammalian cell with an effective amount of a compound of Formula (I), or a pharmaceutically acceptable salt thereof.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

The term “acceptable” with respect to a formulation, composition or ingredient, as used herein, means having no persistent detrimental effect on the general health of the subject being treated.

The terms “effective amount” or “therapeutically effective amount,” as used herein, refer to a sufficient amount of a chemical entity being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt is not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid; organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the terms “subject,” “individual,” or “patient,” are used interchangeably, refer to any animal, including mammals and primates (e.g., human), such as mice, rats, other rodents, rabbits, dogs, cats, swine, pig, cow, goat, cattle, sheep, horses, primates, and humans. In some embodiments, the subject is a human. In some embodiments, the subject has experienced and/or exhibited at least one symptom of the disease or disorder to be treated.

The details of one or more embodiments of the invention are set forth in the accompanying drawings and the description below. Other features and advantages of the invention will be apparent from the description and from the claims.

### DETAILED DESCRIPTION

This disclosure features chemical entities (e.g., a compound or a pharmaceutically acceptable salt, and/or hydrate, and/or cocrystal, and/or drug combination of the compound) that inhibits epidermal growth factor receptor (EGFR, ERBB1) and/or Human epidermal growth factor receptor 2 (HER2, ERBB2). The chemical entities are useful, e.g., for treating a condition, disease or disorder in which increased (e.g., excessive) EGFR and/or HER2 activation contributes to the pathology and/or symptoms and/or progression of the condition, disease or disorder (e.g., cancer) in a subject (e.g., a human). This disclosure also provides compositions containing the same as well as methods of using and making the same.

The compound of Formula (I) is the enantiomer of compound 635 in WO 2022/066734.

### General

In some embodiments, a chemical entity (e.g., a compound that inhibits EGFR and/or HER2, or a pharmaceutically acceptable salt, and/or hydrate, and/or cocrystal, and/or drug combination thereof) is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} a Edition (Pharmaceutical Press, London, U K. 2012).

### Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradable poly(D,L-lactic-co-glycolic acid) [PLGA]-based or poly anhydride-based nanoparticles or microparticles, and nanoporous particle-supported lipid bilayers.

### Dosages

The dosages may be varied depending on the requirement of the patient, the severity of the condition being treated and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/Kg to about 500 mg/Kg (e.g., from about 0.001 mg/Kg to about 200 mg/Kg; from about 0.01 mg/Kg to about 200 mg/Kg; from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg).

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 1 1 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 1 1 months, 12 months, or more. In a further embodiment, a period of during which administration is stopped is for 1 day, 2 days, 3 days, 4 days, 5 days, 6 days, 7 days, 8 days, 9 days, 10 days, 1 1 days, 12 days, 13 days, 14 days, 3 weeks, 4 weeks, 5 weeks, 6 weeks, 7 weeks, 8 weeks, 9 weeks, 10 weeks, 1 1 weeks, 12 weeks, 4 months, 5 months, 6 months, 7 months, 8 months, 9 months, 10 months, 1 1 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

### Methods of Treatment

### Indications

Provided herein are methods for inhibiting epidermal growth factor receptor tyrosine kinase (EGFR) and/or human epidermal growth factor receptor 2 (HER2). For example, provided herein are inhibitors of EGFR useful for treating or preventing diseases or disorders associated with dysregulation of an EGFR gene, an EGFR kinase, or the expression or activity or level of any of the same (i.e., an EGFR-associated disease or disorder), such as a central nervous system diseases, a pulmonary disorder, cardiovascular disease, ischemia, liver disease, a gastrointestinal disorder, a viral or bacterial infection, an inflammatory and/or autoimmune disease, or cancer (e.g., EGFR-associated cancer). In some embodiments, provided herein are inhibitors of HER2 useful for treating or preventing diseases or disorders associated with dysregulation of a HER2 gene, a HER2 kinase, or expression or activity or level of any of the same, such as cancer (e.g., HER2-associated cancer). In some embodiments, provided herein are inhibitors of EGFR and HER2.

An “EGFR inhibitor” as used herein includes any compound exhibiting EGFR inactivation activity (e.g., inhibiting or decreasing). In some embodiments, an EGFR inhibitor can be selective for an EGFR kinase having one or more mutations. For example, an EGFR inhibitor can bind to the adenosine triphosphate (ATP)-binding site in the tyrosine kinase domain. In some embodiments, an EGFR inhibitor is an allosteric inhibitor.

The compound provided herein can inhibit EGFR. In some embodiments, the compound can bind to the EGFR adenosine triphosphate (ATP)-binding site in the tyrosine kinase domain.

The ability of test compounds to act as inhibitors of EGFR may be demonstrated by assays known in the art. The activity of the compounds and compositions provided herein as EGFR inhibitors can be assayed in vitro, in vivo, or in a cell line. In vitro assays include assays that determine inhibition of the kinase and/or ATPase activity. Alternate in vitro assays quantitate the ability of the inhibitor to bind to the protein kinase and can be measured either by radio labelling the compound prior to binding, isolating the compound/kinase complex and determining the amount of radio label bound, or by running a competition experiment where new compounds are incubated with the kinase bound to known radioligands. In some cases, an EGFR inhibitor can be evaluated by its effect on the initial velocity of EGFR tyrosine kinase catalyzed peptide phosphorylation (e.g., Yun et al. Cancer Cell. 2007; 11(3):217-227). In some embodiments, the binding constant of an EGFR inhibitor can be determined using fluorescence kinetics (e.g., Yun et al. Cancer Cell. 2007; 11(3):217-227). Examples of surface plasmon resonance (SPR) binding assays include those disclosed in Li, Shiqing, et al. Cancer cell 7.4 (2005): 301-311. Additional EGFR inhibitor assays can be found, for example, in WO 2019/246541 and WO 2019/165358 both of which are incorporated by reference in their entireties).

Assays can include, for example, proliferation inhibition assays such as those that measure cell growth inhibition, such as an MTS assay or by Cell Titer Glo Luminescent Cell viability assay (Promega®). To perform such an assay, cells are seeded and grown in cell culture plates before being exposed to a test compound for varying durations. Assessment of the viability of the cells following this exposure is then performed. Data are normalized with respect to untreated cells and can be displayed graphically. Growth curves can be fitted using a nonlinear regression model with sigmoidal dose response. As another example, a Western Blot analysis can be used. In such assays cells are seeded and grown in culture plates and then treated with a test compound the following day for varying durations. Cells are washed with PBS and lysed. SDS-PAGE gels are used to separate the lysates which are transferred to nitrocellulose membranes, and probed with appropriate antibodies (e.g., phospho-EGFR(Tyrl 068)(3777), total EGFR (2232), p-Akt(Ser473) (4060), total Akt (9272), p-ERK(Thr202/Tyr204)(4370), total ERK (9102), and HSP90 (SC-7947)).

Additional assays can include, for example, assays based on ALPHALISA TECHNOLOGY® (e.g., see the ALPHALISA® EGF/EGFR binding kit from Promega). Such assays use a luminescent oxygen-channeling chemistry to detect molecules of interest in, for example, buffer, cell culture media, serum, and plasma. For example, a biotinylated EGF is bound to streptavidin-coated Alpha donor beads, and EGFR-Fc is captured by anti-human IgG Fc-specific AlphaLISA acceptor beads. When EGF is bound to EGFR, donor beads and acceptor beads come into close proximity, and the excitation of the donor beads provokes the release of singlet oxygen molecules that triggers a cascade of energy transfers in the acceptor beads. This results in a sharp peak of light emission at 615 nm. Such assays can be used, for example, in competitive binding experiments.

Further examples of assays can include assays based on Sox technology (e.g., see the PHOSPHOSENS® Sox-based Homogeneous, Kinetic or Endpoint/Red Fluorescence-based Assays from ASSAYQUANT®). Such assays utilize chelation-enhanced fluorescence (CHEF) using a sulfonamido-oxine (Sox) chromophore in peptide or protein substrates to create real-time sensors of phosphorylation. See, e.g., U.S. Pat. Nos. 8,586,570 and 6,906,194.

Potency of an EGFR inhibitor as provided herein can be determined by EC₅₀ value. A compound with a lower EC₅₀ value, as determined under substantially similar conditions, is a more potent inhibitor relative to a compound with a higher EC₅₀ value. In some embodiments, the substantially similar conditions comprise determining an EGFR-dependent phosphorylation level, in vitro or in vivo (e.g., in tumor cells, A431 cells, Ba/F3 cells, or 3T3 cells cells expressing a wild type EGFR, a mutant EGFR, or a fragment of any thereof).

Potency of an EGFR inhibitor as provided herein can also be determined by IC₅₀ value. A compound with a lower IC₅₀ value, as determined under substantially similar conditions, is a more potent inhibitor relative to a compound with a higher IC₅₀ value. In some embodiments, the substantially similar conditions comprise determining an EGFR-dependent phosphorylation level, in vitro or in vivo (e.g., in tumor cells, A431 cells, Ba/F3 cells, or 3T3 cells expressing a wild type EGFR, a mutant EGFR, or a fragment of any thereof).

The selectivity between wild type EGFR and EGFR containing one or more mutations as described herein can also be measured using cellular proliferation assays where cell proliferation is dependent on kinase activity. For example, murine Ba/F3 cells transfected with a suitable version of wild type EGFR (such as VIII; containing a wild type EGFR kinase domain), or Ba/F3 cells transfected with L858R/T790M, Del/T790M/L718Q, L858R/T790M/L718Q, L858R/T790M/C797S, Del/T790M/C797S, L858R/T790M/I941R, exon 19 deletion/T790M, or an exon 20 insertion such as V769_D770insX, D770_N771insX, N771_P772insX, P772_H773insX, or H773_V774insX (e.g., A767_V769dupASV, V769_D770insASV, D770_N771insNPG, D770_N771insNPY, D770_N771insSVD, D770_N771insGL, N771_H773dupNPH, N771_P772insN, N771_P772insH, N771_P772insV, P772_H773insDNP, P772_H773insPNP, H773_V774insNPH, H773_V774insH, H773_V774insPH, H773_V774insAH, or P772_H773insPNP) can be used. Proliferation assays are performed at a range of inhibitor concentrations (e.g., 10 μM, 3 μM, 1.1 μM, 330 nM, 110 nM, 33 nM, 11 nM, 3 nM, 1 nM) and an EC₅₀ is calculated.

An alternative method to measure effects on EGFR activity is to assay EGFR phosphorylation. Wildtype or mutant (L858R/T790M, Del/T790M, Del/T790M/L718Q, L858R/T790M/C797S, Del/T790M/C797S, L858R/T790M/I941R, or L858R/T790M/L718Q) EGFR can be transfected into cells which do not normally express endogenous EGFR and the ability of the inhibitor (e.g., using concentrations as above) to inhibit EGFR phosphorylation can be assayed. Cells are exposed to increasing concentrations of inhibitor and stimulated with EGF. The effects on EGFR phosphorylation are assayed by Western Blotting using phospho-specific EGFR antibodies.

In some embodiments, the compounds provided herein can exhibit potent and selective inhibition of EGFR. For example, the compounds provided herein can bind to the EGFR adenosine triphosphate (ATP)-binding site in the tyrosine kinase domain. In some embodiments, the compounds provided herein can exhibit nanomolar potency against an EGFR kinase including an activating mutation or an EGFR inhibitor resistance mutation, including, for example, the resistance mutations in Table 2a and Table 2b (e.g., L747S, D761Y, T790M, and T854A), with minimal activity against related kinases (e.g., wild type EGFR). Inhibition of wild type EGFR can cause undesirable side effects (e.g., diarrhea and skin rashes) that can impact quality of life and compliance. In some cases, the inhibition of wild type EGFR can lead to dose limiting toxicities. See, e.g., Morphy. J. Med. Chem. 2010, 53, 4, 1413-1437 and Peters. J. Med. Chem. 2013, 56, 22, 8955-8971.

In some embodiments, the compound of Formula (I), or a pharmaceutically acceptable salt thereof, can selectively target an EGFR kinase. For example, a compound of Formula (I), or a pharmaceutically acceptable salt thereof, can selectively target an EGFR kinase over another kinase or non-kinase target.

In some embodiments, the compound of Formula (I), or a pharmaceutically acceptable salt thereof, can exhibit greater inhibition of EGFR containing one or more mutations as described herein (e.g., one or more mutations as described in Table 1a and Table 1b) relative to inhibition of wild type EGFR. In some embodiments, the compound of Formula (I), or a pharmaceutically acceptable salt thereof can exhibit at least 2-fold, 3-fold, 5-fold, 10-fold, 25-fold, 50-fold or 100-fold greater inhibition of EGFR containing one or more mutations as described herein relative to inhibition of wild type EGFR. In some embodiments, the compound of Formula (I), or a pharmaceutically acceptable salt thereof, can exhibit up to 1000-fold greater inhibition of EGFR containing one or more mutations as described herein relative to inhibition of wild type EGFR. In some embodiments, the compound of Formula (I), or a pharmaceutically acceptable salt thereof, can exhibit up to 10000-fold greater inhibition of EGFR having a combination of mutations described herein relative to inhibition of wild type EGFR.

In some embodiments, the compound of Formula (I)), or a pharmaceutically acceptable salt thereof, can exhibit from about 2-fold to about 10-fold greater inhibition of EGFR containing one or more mutations as described herein relative to inhibition of wild type EGFR. In some embodiments, the compound of Formula (I), or a pharmaceutically acceptable salt thereof, can exhibit from about 10-fold to about 100-fold greater inhibition of EGFR containing one or more mutations as described herein relative to inhibition of wild type EGFR. In some embodiments, the compound of Formula (I), or a pharmaceutically acceptable salt thereof, can exhibit from about 100-fold to about 1000-fold greater inhibition of EGFR containing one or more mutations as described herein relative to inhibition of wild type EGFR. In some embodiments, the compound of Formula (I), or a pharmaceutically acceptable salt thereof, can exhibit from about 1000-fold to about 10000-fold greater inhibition of EGFR containing one or more mutations as described herein relative to inhibition of wild type EGFR.

In other embodiments, the compound of Formula (I), or a pharmaceutically acceptable salt thereof, in combination with a second EGFR inhibitor can exhibit greater inhibition of EGFR containing one or more mutations as described herein (e.g., one or more mutations as described in Table 1a and Table 1b) relative to inhibition of wild type EGFR. In some embodiments, the compound of Formula (I), or a pharmaceutically acceptable salt thereof, in combination with a second EGFR inhibitor can exhibit at least 2-fold, 3-fold, 5-fold, 10-fold, 25-fold, 50-fold or 100-fold greater inhibition of EGFR containing one or more mutations as described herein relative to inhibition of wild type EGFR. In some embodiments, the compound of Formula (I), or a pharmaceutically acceptable salt thereof, in combination with a second EGFR inhibitor can exhibit up to 1000-fold greater inhibition of EGFR containing one or more mutations as described herein relative to inhibition of wild type EGFR. In some embodiments, the compound of Formula (I), or a pharmaceutically acceptable salt thereof, in combination with a second EGFR inhibitor can exhibit up to 10000-fold greater inhibition of EGFR having a combination of mutations described herein relative to inhibition of wild type EGFR.

In other embodiments, the compound of Formula (I), or a pharmaceutically acceptable salt thereof, in combination with a second EGFR inhibitor can exhibit from about 2-fold to about 10-fold greater inhibition of EGFR containing one or more mutations as described herein relative to inhibition of wild type EGFR. In some embodiments, the compound of Formula (I), or a pharmaceutically acceptable salt thereof, in combination with a second EGFR inhibitor can exhibit from about 10-fold to about 100-fold greater inhibition of EGFR containing one or more mutations as described herein relative to inhibition of wild type EGFR. In some embodiments, the compound of Formula (I), or a pharmaceutically acceptable salt thereof, in combination with a second EGFR inhibitor can exhibit from about 100-fold to about 1000-fold greater inhibition of EGFR containing one or more mutations as described herein relative to inhibition of wild type EGFR. In some embodiments, the compound of Formula (I), or a pharmaceutically acceptable salt thereof, in combination with a second EGFR inhibitor can exhibit from about 1000-fold to about 10000-fold greater inhibition of EGFR containing one or more mutations as described herein relative to inhibition of wild type EGFR.

The compound of Formula (I), or a pharmaceutically acceptable salt or solvate thereof, are useful for treating diseases and disorders which can be treated with an EGFR inhibitor, such as EGFR-associated diseases and disorders, e.g., central nervous system diseases (e.g., neurodegenerative diseases), pulmonary disorders, cardiovascular disease, ischemia, liver disease, gastrointestinal disorders, viral or bacterial infections, inflammatory and/or autoimmune diseases (e.g., psoriasis and atopic dermatitis), and proliferative disorders such as cancers, including hematological cancers and solid tumors (e.g., advanced solid tumors).

A “HER2 inhibitor” as used herein includes any compound exhibiting HER2 inactivation activity (e.g., inhibiting or decreasing). In some embodiments, a HER2 inhibitor can be selective for a HER2 kinase having one or more mutations. In some embodiments, a HER2 inhibitor can bind to the HER2 adenosine triphosphate (ATP)-binding site in the tyrosine kinase domain.

The compound provided herein can inhibit HER2. For example, the compound can bind to the HER2 adenosine triphosphate (ATP)-binding site in the tyrosine kinase domain. In some embodiments, the compounds provided herein can inhibit wild type HER2. In some embodiments, the compounds provided herein can inhibit HER2 having one or more mutations as described herein.

The ability of test compounds to act as inhibitors of HER2 may be demonstrated by assays known in the art. The activity of the compounds or compositions provided herein as HER2 inhibitors can be assayed in vitro, in vivo, or in a cell line. In vitro assays include assays that determine inhibition of the kinase and/or ATPase activity. Alternate in vitro assays quantitate the ability of the inhibitor to bind to the protein kinase and can be measured either by radio labelling the compound prior to binding, isolating the compound/kinase complex and determining the amount of radio label bound, or by running a competition experiment where new compounds are incubated with the kinase bound to known radioligands. In some cases, a HER2 inhibitor can be evaluated by its effect on the initial velocity of HER2 tyrosine kinase catalyzed peptide phosphorylation (e.g., Yun et al. Cancer Cell. 2007; 11(3):217-227). For example, an assay that indirectly measures ADP formed from the HER2 kinase reaction can be used (see, e.g., ATP/NADH coupled assay systems and luminescent kinase assays such as ADP-GLO™ Kinase Assay from Promega). See, e.g., Hanker et al. Cancer Discov. 2017 June; 7(6):575-585; Robichaux et al. Nat Med. 2018 May; 24(5): 638-646; and Yun et al. Proc Natl Acad Sci USA. 2008 Feb. 12; 105(6):2070-5. In some embodiments, an assay that detects substrate phosphorylation using a labeled anti-phospho-tyrosine antibody can be used (see, e.g., Rabindran et al. Cancer Res. 2004 Jun. 1; 64(11):3958-65). In some embodiments, the binding constant of a HER2 inhibitor can be determined using fluorescence kinetics (e.g., Yun et al. Cancer Cell. 2007; 11(3):217-227). Examples of SPR binding assays include those disclosed in Li, Shiqing, et al. Cancer cell 7.4 (2005): 301-311. In some embodiments, covalent binding of a HER2 inhibitor to HER2 can be detected using mass spectrometry, see, e.g., Irie et al. Mol Cancer Ther. 2019 April; 18(4):733-742. Additional HER2 inhibitor assays can be found, for example, in U.S. Pat. No. 9,920,060, WO 2019/241715, and U.S. Publication No. 2017/0166598, each of which are incorporated by reference in their entireties.

Potency of a HER2 inhibitor as provided herein can be determined by EC₅₀ value. A compound with a lower EC₅₀ value, as determined under substantially similar conditions, is a more potent inhibitor relative to a compound with a higher EC₅₀ value. In some embodiments, the substantially similar conditions comprise determining an HER2-dependent phosphorylation level, in vitro or in vivo (e.g., in tumor cells or Ba/F3 cells expressing a wild type HER2, a mutant HER2, or a fragment of any thereof).

Potency of an HER2 inhibitor as provided herein can also be determined by IC₅₀ value. A compound with a lower IC₅₀ value, as determined under substantially similar conditions, is a more potent inhibitor relative to a compound with a higher IC₅₀ value. In some embodiments, the substantially similar conditions comprise determining an HER2-dependent phosphorylation level, in vitro or in vivo (e.g., in tumor cells or Ba/F3 cells expressing a wild type HER2, a mutant HER2, or a fragment of any thereof).

Assays can include, for example, proliferation inhibition assays such as those that measure cell growth inhibition, such as an MTS assay or by Cell Titer Glo Luminescent Cell viability assay (Promega®). To perform such an assay, cells are seeded and grown in cell culture plates before being exposed to a test compound for varying durations. Assessment of the viability of the cells following this exposure is then performed. Data are normalized with respect to untreated cells and can be displayed graphically. Growth curves can be fitted using a nonlinear regression model with sigmoidal dose response. As another example, a Western Blot analysis can be used. In such assays cells are seeded and grown in culture plates and then treated with a test compound the following day for varying durations. Cells are washed with PBS and lysed. SDS-PAGE gels are used to separate the lysates which are transferred to nitrocellulose membranes, and probed with appropriate antibodies (e.g., phospho-HER2(Tyr1248)(2247), phospho-EGFR-Tyr1173 phospho-HER2-Tyr877, phospho-HER2-Tyr1221, total HER2, phospho-AKT-Thr308, phospho-AKT-Ser374, total AKT, phospho-p44/42 MAPK-Thr202/Tyr204, and p44/42 MAPK).

The selectivity between wild type HER2 and HER2 containing one or more mutations as described herein can also be measured using cellular proliferation assays where cell proliferation is dependent on kinase activity. For example, murine Ba/F3 cells transfected with a suitable version of wild type HER2, or Ba/F3 cells transfected with HER2 having one or more mutations such as S310F, S310Y, R678Q, R678W, R678P, I767M, V773M, V777L, V842I, M774AYVM, M774del insWLV, A775_G776insYVMA, A775_G776insAVMA, A775_G776insSVMA, A775_G776insVAG, A775insV G776C, A775_G776insI, G776del insVC2, G776del insVV, G776del insLC, G776C V777insC, G776C V777insV, V777 G778insCG, G778 S779insCPG, or P780_Y781insGSP can be used. Proliferation assays are performed at a range of inhibitor concentrations (e.g., 10 μM, 3 μM, 1.1 μM, 330 nM, 110 nM, 33 nM, 11 nM, 3 nM, 1 nM) and an EC₅₀ is calculated.

An alternative method to measure effects on HER2 activity is to assay HER2 phosphorylation. Wildtype or mutant (S310F, S310Y, R678Q, R678W, R678P, I767M, V773M, V777L, V842I, M774AYVM, M774del insWLV, A775_G776insYVMA, A775_G776insAVMA, A775_G776insSVMA, A775 G776insVAG, A775insV G776C, A775_G776insI, G776del insVC2, G776del insVV, G776del insLC, G776C V777insC, G776C V777insV, V777 G778insCG, G778 S779insCPG, or P780 Y781insGSP) HER2 can be transfected into cells which do not normally express endogenous HER2 and the ability of the inhibitor (e.g., using concentrations as above) to inhibit HER2 phosphorylation can be assayed. Cells are exposed to increasing concentrations of inhibitor and stimulated with EGF. The effects on HER2 phosphorylation are assayed by Western Blotting using phospho-specific HER2 antibodies.

In some embodiments, the compound provided herein can exhibit potent and selective inhibition of HER2. For example, the compounds provided herein can bind to the HER2 adenosine triphosphate (ATP)-binding site in the tyrosine kinase domain. In some embodiments, the compounds provided herein can exhibit nanomolar potency against a HER2 kinase including an activating mutation or a HER2 inhibitor resistance mutation, including, for example, exon 20 insertions and/or the resistance mutations in Table 5 (e.g., L755S, L755P, T798I, and T798M), with minimal activity against related kinases (e.g., wild type EGFR).

In some embodiments, the compound of Formula (I), or a pharmaceutically acceptable salt thereof, can selectively target a HER2 kinase. For example, the compound of Formula (I), or a pharmaceutically acceptable salt thereof, can selectively target a HER2 kinase over another kinase (e.g., wild type EGFR) or non-kinase target. It can be desirable to selectively target a HER2 kinase over a wild type EGFR kinase due to undesirable side effects (e.g., diarrhea and skin rashes) that can impact quality of life and compliance. See, e.g., Morphy. J. Med. Chem. 2010, 53, 4, 1413-1437 and Peters. J. Med. Chem. 2013, 56, 22, 8955-8971.

In some embodiments, the compound of Formula (I), or a pharmaceutically acceptable salt thereof, can exhibit greater inhibition of wild type HER2 or HER2 containing one or more mutations as described herein (e.g., one or more mutations as described in Table 3) relative to inhibition of another kinase (e.g., wild type EGFR) or non-kinase target. In some embodiments, the compound of Formula (I), or a pharmaceutically acceptable salt thereof can exhibit at least 2-fold, 3-fold, 5-fold, 10-fold, 25-fold, 50-fold or 100-fold greater inhibition of wild type HER2 or HER2 containing one or more mutations as described herein relative to inhibition of another kinase (e.g., wild type EGFR) or non-kinase target. In some embodiments, the compound of Formula (I), or a pharmaceutically acceptable salt thereof, can exhibit up to 1000-fold greater inhibition of wild type HER2 or HER2 containing one or more mutations as described herein relative to inhibition of another kinase (e.g., wild type EGFR) or non-kinase target. In some embodiments, the compound of Formula (I), or a pharmaceutically acceptable salt thereof, can exhibit up to 10000-fold greater inhibition of wild type HER2 or HER2 having a combination of mutations described herein relative to inhibition of another kinase (e.g., wild type EGFR) or non-kinase target.

In some embodiments, the compound of Formula (I), or a pharmaceutically acceptable salt thereof, can exhibit from about 2-fold to about 10-fold greater inhibition of wild type HER2 or HER2 containing one or more mutations as described herein relative to inhibition of another kinase (e.g., wild type EGFR) or non-kinase target. In some embodiments, the compound of Formula (I), or a pharmaceutically acceptable salt thereof, can exhibit from about 10-fold to about 100-fold greater inhibition of wild type HER2 or containing one or more mutations as described herein relative to inhibition of another kinase (e.g., wild type EGFR) or non-kinase target. In some embodiments, the compound of Formula (I), or a pharmaceutically acceptable salt thereof, can exhibit from about 100-fold to about 1000-fold greater inhibition of wild type HER2 or HER2 containing one or more mutations as described herein relative to inhibition of another kinase (e.g., wild type EGFR) or non-kinase target. In some embodiments, the compound of Formula (I), or a pharmaceutically acceptable salt thereof, can exhibit from about 1000-fold to about 10000-fold greater inhibition of wild type HER2 or HER2 containing one or more mutations as described herein relative to inhibition of another kinase (e.g., wild type EGFR) or non-kinase target.

In other embodiments, the compound of Formula (I), or a pharmaceutically acceptable salt thereof, in combination with a second HER2 inhibitor can exhibit greater inhibition of wild type HER2 or HER2 containing one or more mutations as described herein (e.g., one or more mutations as described in Table 3) relative to inhibition of another kinase (e.g., wild type EGFR) or non-kinase target. In some embodiments, the compound of Formula (I), or a pharmaceutically acceptable salt thereof, in combination with a second HER2 inhibitor can exhibit at least 2-fold, 3-fold, 5-fold, 10-fold, 25-fold, 50-fold or 100-fold greater inhibition of wild type HER2 or HER2 containing one or more mutations as described herein relative to inhibition of another kinase (e.g., wild type EGFR) or non-kinase target. In some embodiments, the compound of Formula (I), or a pharmaceutically acceptable salt thereof, in combination with a second HER2 inhibitor can exhibit up to 1000-fold greater inhibition of wild type HER2 or HER2 containing one or more mutations as described herein relative to inhibition of another kinase (e.g., wild type EGFR) or non-kinase target. In some embodiments, the compound of Formula (I), or a pharmaceutically acceptable salt thereof, in combination with a second HER2 inhibitor can exhibit up to 10000-fold greater inhibition of wild type HER2 or HER2 having a combination of mutations described herein relative to inhibition of another kinase (e.g., wild type EGFR) or non-kinase target.

In other embodiments, the compound of Formula (I), or a pharmaceutically acceptable salt thereof, in combination with a second HER2 inhibitor can exhibit from about 2-fold to about 10-fold greater inhibition of wild type HER2 or HER2 containing one or more mutations as described herein relative to inhibition of another kinase (e.g., wild type EGFR) or non-kinase target. In some embodiments, the compound of Formula (I), or a pharmaceutically acceptable salt thereof, in combination with a second HER2 inhibitor can exhibit from about 10-fold to about 100-fold greater inhibition of wild type HER2 or HER2 containing one or more mutations as described herein relative to inhibition of another kinase (e.g., wild type EGFR) or non-kinase target. In some embodiments, the compound of Formula (I), or a pharmaceutically acceptable salt thereof, in combination with a second HER2 inhibitor can exhibit from about 100-fold to about 1000-fold greater inhibition of wild type HER2 or HER2 containing one or more mutations as described herein relative to inhibition of another kinase (e.g., wild type EGFR) or non-kinase target. In some embodiments, the compound of Formula (I), or a pharmaceutically acceptable salt thereof, in combination with a second HER2 inhibitor can exhibit from about 1000-fold to about 10000-fold greater inhibition of wild type HER2 or HER2 containing one or more mutations as described herein relative to inhibition of another kinase (e.g., wild type EGFR) or non-kinase target.

The compounds of Formula (I), or pharmaceutically acceptable salt or solvate thereof, is useful for treating diseases and disorders which can be treated with a HER2 inhibitor, such as HER2-associated diseases and disorders, e.g., proliferative disorders such as cancers (e.g., a HER2-associated cancer), including hematological cancers and solid tumors (e.g., advanced solid tumors).

In some embodiments, the compound provided herein can also inhibit EGFR and HER2 as described herein.

In some embodiments, the compound provided herein can exhibit potent and selective inhibition of EGFR and HER2. In some embodiments, the compound provided herein can exhibit nanomolar potency against an EGFR kinase having one or more mutations, including, for example, one or more of the mutations in Tables 1a, 1b, 2a and 2b, and a HER2 kinase having one or more mutations, including, for example, the mutations in Table 3, with minimal activity against related kinases (e.g., wild type EGFR).

In some embodiments, the compound of Formula (I), or a pharmaceutically acceptable salt thereof, can selectively target an EGFR and a HER2 kinase. For example, the compound of Formula (I), or a pharmaceutically acceptable salt thereof, can selectively target an EGFR kinase and a HER2 kinase over another kinase or non-kinase target.

In some embodiments, the compound of Formula (I), or a pharmaceutically acceptable salt thereof, can exhibit greater inhibition of EGFR containing one or more mutations as described herein and wild type HER2 or HER2 containing one or more mutations as described herein (e.g., one or more mutations as described in Tables 3-5) relative to inhibition of another kinase (e.g., wild type EGFR) or non-kinase target. In some embodiments, the compound of Formula (I), or a pharmaceutically acceptable salt thereof can exhibit at least 2-fold, 3-fold, 5-fold, 10-fold, 25-fold, 50-fold or 100-fold greater inhibition of EGFR containing one or more mutations as described herein and wild type HER2 or HER2 containing one or more mutations as described herein relative to inhibition of another kinase (e.g., wild type EGFR) or non-kinase target. In some embodiments, the compound of Formula (I), or a pharmaceutically acceptable salt thereof, can exhibit up to 1000-fold greater inhibition of EGFR containing one or more mutations as described herein and wild type HER2 or HER2 containing one or more mutations as described herein relative to inhibition of another kinase (e.g., wild type EGFR) or non-kinase target. In some embodiments, the compound of Formula (I), or a pharmaceutically acceptable salt thereof, can exhibit up to 10000-fold greater inhibition of EGFR containing one or more mutations as described herein and wild type HER2 or HER2 having one or more mutations described herein relative to inhibition of another kinase (e.g., wild type EGFR) or non-kinase target.

In some embodiments, the compound of Formula (I)), or a pharmaceutically acceptable salt thereof, can exhibit from about 2-fold to about 10-fold greater inhibition of EGFR containing one or more mutations as described herein and wild type HER2 or HER2 containing one or more mutations as described herein relative to inhibition of another kinase (e.g., wild type EGFR) or non-kinase target. In some embodiments, the compound of Formula (I), or a pharmaceutically acceptable salt thereof, can exhibit from about 10-fold to about 100-fold greater inhibition of EGFR containing one or more mutations as described herein and wild type HER2 or HER2 containing one or more mutations as described herein relative to inhibition of another kinase (e.g., wild type EGFR) or non-kinase target. In some embodiments, the compound of Formula (I), or a pharmaceutically acceptable salt thereof, can exhibit from about 100-fold to about 1000-fold greater inhibition of EGFR containing one or more mutations as described herein and wild type HER2 or HER2 containing one or more mutations as described herein relative to inhibition of another kinase (e.g., wild type EGFR) or non-kinase target. In some embodiments, the compound of Formula (I), or a pharmaceutically acceptable salt thereof, can exhibit from about 1000-fold to about 10000-fold greater inhibition of EGFR containing one or more mutations as described herein and wild type HER2 or HER2 containing one or more mutations as described herein relative to inhibition of another kinase (e.g., wild type EGFR) or non-kinase target.

In other embodiments, the compound of Formula (I), or a pharmaceutically acceptable salt thereof, in combination with a second EGFR and/or second HER2 inhibitor can exhibit greater inhibition of EGFR containing one or more mutations as described herein and wild type HER2 or HER2 containing one or more mutations as described herein (e.g., one or more mutations as described in Table 3) relative to inhibition of another kinase (e.g., wild type EGFR) or non-kinase target. In some embodiments, the compound of Formula (I), or a pharmaceutically acceptable salt thereof, in combination with a second EGFR and/or second HER2 inhibitor can exhibit at least 2-fold, 3-fold, 5-fold, 10-fold, 25-fold, 50-fold or 100-fold greater inhibition of EGFR containing one or more mutations as described herein and wild type HER2 or HER2 containing one or more mutations as described herein relative to inhibition of another kinase (e.g., wild type EGFR) or non-kinase target. In some embodiments, the compound of Formula (I), or a pharmaceutically acceptable salt thereof, in combination with a second EGFR and/or second HER2 inhibitor can exhibit up to 1000-fold greater inhibition of EGFR containing one or more mutations as described herein and wild type HER2 or HER2 containing one or more mutations as described herein relative to inhibition of another kinase (e.g., wild type EGFR) or non-kinase target. In some embodiments, thea compound of Formula (I), or a pharmaceutically acceptable salt thereof, in combination with a second EGFR and/or HER2 inhibitor can exhibit up to 10000-fold greater inhibition of EGFR containing one or more mutations as described herein and wild type HER2 or HER2 having a combination of mutations described herein relative to inhibition of another kinase (e.g., wild type EGFR) or non-kinase target.

In other embodiments, the compound of Formula (I)), or a pharmaceutically acceptable salt thereof, in combination with a second EGFR and/or second HER2 inhibitor can exhibit from about 2-fold to about 10-fold greater inhibition of EGFR containing one or more mutations as described herein and HER2 containing one or more mutations as described herein relative to inhibition of another kinase (e.g., wild type EGFR) or non-kinase target. In some embodiments, the compound of Formula (I), or a pharmaceutically acceptable salt thereof, in combination with a second EGFR and/or second HER2 inhibitor can exhibit from about 10-fold to about 100-fold greater inhibition of EGFR containing one or more mutations as described herein and HER2 containing one or more mutations as described herein relative to inhibition of another kinase (e.g., wild type EGFR) or non-kinase target. In some embodiments, the compound of Formula (I), or a pharmaceutically acceptable salt thereof, in combination with a second EGFR and/or second HER2 inhibitor can exhibit from about 100-fold to about 1000-fold greater inhibition of EGFR containing one or more mutations as described herein and second HER2 containing one or more mutations as described herein relative to inhibition of another kinase (e.g., wild type EGFR) or non-kinase target. In some embodiments, the compound of Formula (I), or a pharmaceutically acceptable salt thereof, in combination with a second EGFR and/or second HER2 inhibitor can exhibit from about 1000-fold to about 10000-fold greater inhibition of EGFR containing one or more mutations as described herein and HER2 containing one or more mutations as described herein relative to inhibition of another kinase (e.g., wild type EGFR) or non-kinase target.

Also provided herein are methods for inhibiting a BUB (budding uninhibited by benzimidazole, BUB1-3) kinase. For example, provided herein are inhibitors of BUB1 kinase useful for treating or preventing diseases or disorders associated with enhanced uncontrolled proliferative cellular processes such as, for example, cancer, inflammation, arthritis, viral diseases, cardiovascular diseases, or fungal diseases. See, for example, WO 2013/050438, WO 2013/092512, WO 2013/167698, WO 2014/147203, WO 2014/147204, WO 2014/202590, WO 2014/202588, WO 2014/202584, WO 2014/202583, WO 2015/063003, WO2015/193339, WO 2016/202755, and WO 2017/021348. In some embodiments, the disease or disorder is cancer.

A “BUB1 inhibitor” as used herein includes any compound exhibiting BUB1 inactivation activity (e.g., inhibiting or decreasing). In some embodiments, a BUB1 inhibitor can be selective for BUB1 over other kinases (e.g., wildtype EGFR).

The compounds provided herein can inhibit a Bub kinase. In some embodiments, the compounds provided herein can inhibit BUB1 kinase.

The ability of test compounds to act as inhibitors of BUB1 may be demonstrated by assays known in the art. The activity of the compounds and compositions provided herein as BUB1 inhibitors can be assayed in vitro, in vivo, or in a cell line. In vitro assays include assays that determine inhibition of the kinase. For example, BUB1 inhibition of a compound provided herein can be determined using a time-resolved fluorescence energy transfer (TR-FRET) assay which measures phosphorylation of a synthetic peptide (e.g., Biotin-AHX-VLLPKKSFAEPG (C-terminus in amide form) by the (recombinant) catalytic domain of human BUB1 (amino acids 704-1085), expressed in Hi5 insect cells with an N-terminal His6-tag and purified by affinity- (Ni-NTA) and size exclusion chromatography. See, for example, WO 2017/021348. In addition, BUB1 activity can be determined at a high ATP concentration using a BUB1 TR-FRET high ATP kinase assay using similar methods as those described above. See, e.g. WO 2019/081486.

In some embodiments, the compounds provided herein exhibit central nervous system (CNS) penetrance. For example, such compounds can be capable of crossing the blood brain barrier (BBB) and inhibiting an EGFR and/or HER2 kinase in the brain and/or other CNS structures. In some embodiments, the compounds provided herein are capable of crossing the blood brain barrier in a therapeutically effective amount. For example, treatment of a patient with cancer (e.g., an EGFR-associated cancer or a HER2-associated cancer such as an EGFR- or HER2-associated brain or CNS cancer or an EGFR-associated or a HER2-associated cancer that has metastasized to the brain or CNS) can include administration (e.g., oral administration) of the compound to the patient.

The ability of the compounds described herein, to cross the BBB can be demonstrated by assays known in the art. Such assays include BBB models such as the transwell system, the hollow fiber (dynamic in vitro BBB) model, other microfluidic BBB systems, the BBB spheroid platform, and other cell aggregate-based BBB models. See, e.g., Cho et al. Nat Commun. 2017; 8: 15623; Bagchi et al. Drug Des Devel Ther. 2019; 13: 3591-3605; Gastfriend et al. Curr Opin Biomed Eng. 2018 March; 5: 6-12; and Wang et al. Biotechnol Bioeng. 2017 January; 114(1): 184-194. In some embodiments, the compounds described herein, are fluorescently labeled, and the fluorescent label can be detected using microscopy (e.g., confocal microscopy). In some such embodiments, the ability of the compound to penetrate the surface barrier of the model can be represented by the fluorescence intensity at a given depth below the surface. In some assays, such as a calcein-AM-based assay, the fluorescent label is non-fluorescent until it permeates live cells and is hydrolyzed by intracellular esterases to produce a fluorescent compound that is retained in the cell and can be quantified with a spectrophotometer. Non-limiting examples of fluorescent labels that can be used in the assays described herein include Cy5, rhodamine, infrared IRDye® CW-800 (LICOR #929-71012), far-red IRDye® 650 (LICOR #929-70020), sodium fluorescein (Na—F), lucifer yellow (LY), 5′carboxyfluorescein, and calcein-acetoxymethylester (calcein-AM). In some embodiments, the BBB model (e.g., the tissue or cell aggregate) can be sectioned, and a compound described herein can be detected in one or more sections using mass spectrometry (e.g., MALDI-MSI analyses). In some embodiments, the ability of a compound described herein to cross the BBB through a transcellular transport system, such as receptor-mediated transport (RMT), carrier-mediated transport (CMT), or active efflux transport (AET), can be demonstrated by assays known in the art. See, e.g., Wang et al. Drug Deliv. 2019; 26(1): 551-565. In some embodiments, assays to determine if compounds can be effluxed by the P-glycoprotein (Pgp) include monolayer efflux assays in which movement of compounds through Pgp is quantified by measuring movement of digoxin, a model Pgp substrate (see, e.g., Doan et al. 2002. J Pharmacol Exp Ther. 303(3):1029-1037). Alternative in vivo assays to identify compounds that pass through the blood-brain barriers include phage-based systems (see, e.g., Peng et al. 2019. ChemRxiv. Preprint doi.org/10.26434/chemrxiv.8242871.v1). In some embodiments, binding of the compounds described herein to brain tissue is quantified. For example, a brain tissue binding assay can be performed using equilibrium dialysis, and the fraction of a compound described herein unbound to brain tissue can be detected using LC-MS/MS (Cyprotex: Brain Tissue Binding Assay www.cyprotex.com/admepk/protein_binding/brain-tissue-binding).

The compound of Formula (I), or pharmaceutically acceptable salts or solvates thereof, are useful for treating diseases and disorders which can be treated with an EGFR inhibitor, a HER2 inhibitor, a dual EGFR and HER2 inhibitor, and/or a BUB1 inhibitor, such as those described herein, e.g., cancer. Accordingly, provided herein is a method for treating a disease or disorder as provided herein in a subject in need thereof, the method comprising administering to the subject a therapeutically effective amount of the compound of Formula (I), or a pharmaceutically acceptable salt or solvate thereof. In some embodiments, the disease or disorder is cancer.

In some embodiments, the subject has been identified or diagnosed as having a cancer with a dysregulation of an EGFR gene, an EGFR protein, or expression or activity, or level of any of the same (an EGFR-associated cancer) (e.g., as determined using a regulatory agency-approved, e.g., FDA-approved, assay or kit). In some embodiments, the subject has a tumor that is positive for a dysregulation of an EGFR gene, an EGFR protein, or expression or activity, or level of any of the same (e.g., as determined using a regulatory agency-approved assay or kit). For example, the subject has a tumor that is positive for a mutation as described in Table 1a and Table 1b. The subject can be a subject with a tumor(s) that is positive for a dysregulation of an EGFR gene, an EGFR protein, or expression or activity, or level of any of the same (e.g., identified as positive using a regulatory agency-approved, e.g., FDA-approved, assay or kit). The subject can be a subject whose tumors have a dysregulation of an EGFR gene, an EGFR protein, or expression or activity, or a level of the same (e.g., where the tumor is identified as such using a regulatory agency-approved, e.g., FDA-approved, kit or assay). In some embodiments, the subject is suspected of having an EGFR-associated cancer. In some embodiments, the subject has a clinical record indicating that the subject has a tumor that has a dysregulation of an EGFR gene, an EGFR protein, or expression or activity, or level of any of the same (and optionally the clinical record indicates that the subject should be treated with any of the compositions provided herein).

In some embodiments, the subject has been identified or diagnosed as having a cancer with a dysregulation of a HER2 gene, a HER2 protein, or expression or activity, or level of any of the same (a HER2-associated cancer) (e.g., as determined using a regulatory agency-approved, e.g., FDA-approved, assay or kit). In some embodiments, the subject has a tumor that is positive for a dysregulation of a HER2 gene, a HER2 protein, or expression or activity, or level of any of the same (e.g., as determined using a regulatory agency-approved assay or kit). For example, the subject has a tumor that is positive for a mutation as described in Table 3. The subject can be a subject with a tumor(s) that is positive for a dysregulation of a HER2 gene, a HER2 protein, or expression or activity, or level of any of the same (e.g., identified as positive using a regulatory agency-approved, e.g., FDA-approved, assay or kit). The subject can be a subject whose tumors have a dysregulation of a HER2 gene, a HER2 protein, or expression or activity, or a level of the same (e.g., where the tumor is identified as such using a regulatory agency-approved, e.g., FDA-approved, kit or assay). In some embodiments, the subject is suspected of having a HER2-associated cancer. In some embodiments, the subject has a clinical record indicating that the subject has a tumor that has a dysregulation of a HER2 gene, a HER2 protein, or expression or activity, or level of any of the same (and optionally the clinical record indicates that the subject should be treated with any of the compositions provided herein).

In some embodiments, the subject is a pediatric subject.

The term “pediatric subject” as used herein refers to a subject under the age of 21 years at the time of diagnosis or treatment. The term “pediatric” can be further divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric subject is from birth through the first 28 days of life, from 29 days of age to less than two years of age, from two years of age to less than 12 years of age, or 12 years of age through 21 years of age (up to, but not including, the twenty-second birthday). In some embodiments, a pediatric subject is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age.

In certain embodiments, the compound of Formula (I), or pharmaceutically acceptable salts or solvates thereof, are useful for preventing diseases and disorders as defined herein (for example, autoimmune diseases, inflammatory diseases, pulmonary disorders, cardiovascular disease, ischemia, liver disease, gastrointestinal disorders, viral or bacterial infections, central nervous system diseases (e.g., neurodegenerative diseases), and cancer). The term “preventing” as used herein means to delay the onset, recurrence or spread, in whole or in part, of the disease or condition as described herein, or a symptom thereof.

The term “EGFR-associated disease or disorder” as used herein refers to diseases or disorders associated with or having a dysregulation of an EGFR gene, an EGFR kinase (also called herein an EGFR kinase protein), or the expression or activity or level of any (e.g., one or more) of the same (e.g., any of the types of dysregulation of an EGFR gene, an EGFR kinase, an EGFR kinase domain, or the expression or activity or level of any of the same described herein). Non-limiting examples of an EGFR-associated disease or disorder include, for example, cancer, a central nervous system disease, a pulmonary disorder, cardiovascular disease, ischemia, liver disease, a gastrointestinal disorder, a viral or bacterial infection, and an inflammatory and/or autoimmune disease (e.g., psoriasis, eczema, atopic dermatitis, and atherosclerosis).

In some embodiments of any of the methods or uses described herein, the inflammatory and/or autoimmune disease is selected from arthritis, systemic lupus erythematosus, atherosclerosis, and skin related disorders such as psoriasis, eczema, and atopic dermatitis. See, e.g., Wang et al. Am J Transl Res. 2019; 11(2): 520-528; Starosyla et al. World J Pharmacol. Dec. 9, 2014; 3(4): 162-173; Choi et al. Biomed Res Int. 2018 May 15; 2018:9439182; and Wang et al. Sci Rep. 2017; 7: 45917.

In some embodiments of any of the methods or uses described herein, the central nervous system disease is a neurodegenerative disease. In some embodiments, the central nervous system disease is selected from Alzheimer's disease, Parkinson's disease, Huntington's disease, amyotrophic lateral sclerosis, spinal cord injury, peripheral neuropathy, brain ischemia, and a psychiatric disorder such as schizophrenia. See, e.g., Iwakura and Nawa. Front Cell Neurosci. 2013 Feb. 13; 7:4; and Chen et al. Sci Rep. 2019 Feb. 21; 9(1):2516.

The term “EGFR-associated cancer” as used herein refers to cancers associated with or having a dysregulation of an EGFR gene, an EGFR kinase (also called herein an EGFR kinase protein), or expression or activity, or level of any of the same. Non-limiting examples of an EGFR-associated cancer are described herein.

The phrase “dysregulation of an EGFR gene, an EGFR kinase, or the expression or activity or level of any of the same” refers to a genetic mutation (e.g., a mutation in an EGFR gene that results in the expression of an EGFR protein that includes a deletion of at least one amino acid as compared to a wild type EGFR protein, a mutation in an EGFR gene that results in the expression of an EGFR protein with one or more point mutations as compared to a wild type EGFR protein, a mutation in an EGFR gene that results in the expression of an EGFR protein with at least one inserted amino acid as compared to a wild type EGFR protein, a gene duplication that results in an increased level of EGFR protein in a cell, or a mutation in a regulatory sequence (e.g., a promoter and/or enhancer) that results in an increased level of EGFR protein in a cell), an alternative spliced version of an EGFR mRNA that results in an EGFR protein having a deletion of at least one amino acid in the EGFR protein as compared to the wild type EGFR protein), or increased expression (e.g., increased levels) of a wild type EGFR kinase in a mammalian cell due to aberrant cell signaling and/or dysregulated autocrine/paracrine signaling (e.g., as compared to a control non-cancerous cell). As another example, a dysregulation of an EGFR gene, an EGFR protein, or expression or activity, or level of any of the same, can be a mutation in an EGFR gene that encodes an EGFR protein that is constitutively active or has increased activity as compared to a protein encoded by an EGFR gene that does not include the mutation. Non-limiting examples of EGFR kinase protein point mutations/insertions/deletions are described in Table 1a and Table 1b. Additional examples of EGFR kinase protein mutations (e.g., point mutations) are EGFR inhibitor resistance mutations (e.g., EGFR inhibitor mutations). Non-limiting examples of EGFR inhibitor resistance mutations are described in Table 2a and Table 2b. For example, the one or more EGFR inhibitor resistance mutations can include a substitution at amino acid position 718, 747, 761, 790, 797, or 854 (e.g., L718Q, L747S, D761Y, T790M, C797S, or T854A). Such mutation and overexpression is associated with the development of a variety of cancers (Shan et al., Cell 2012, 149(4) 860-870).

In some embodiments, dysregulation of an EGFR gene, an EGFR kinase, or the expression or activity or level of any of the same can be caused by an activating mutation in an EGFR gene. In some embodiments, dysregulation of an EGFR gene, an EGFR kinase, or the expression or activity or level of any of the same can be caused by a genetic mutation that results in the expression of an EGFR kinase that has increased resistance to an EGFR inhibitor, a tyrosine kinase inhibitor (TKI), and/or a multi-kinase inhibitor (MKI), e.g., as compared to a wild type EGFR kinase (see, e.g., the amino acid substitutions in Table 2a and Table 2b). In some embodiments, dysregulation of an EGFR gene, an EGFR kinase, or the expression or activity or level of any of the same can be caused by a mutation in a nucleic acid encoding an altered EGFR protein (e.g., an EGFR protein having a mutation (e.g., a primary mutation)) that results in the expression of an altered EGFR protein that has increased resistance to inhibition by an EGFR inhibitor, a tyrosine kinase inhibitor (TKI), and/or a multi-kinase inhibitor (MKI), e.g., as compared to a wild type EGFR kinase (see, e.g., the amino acid substitutions in Table 2a and Table 2b). The exemplary EGFR kinase point mutations, insertions, and deletions shown in Tables 1a, 1b, 2a and 2b can be caused by an activating mutation and/or can result in the expression of an EGFR kinase that has increased resistance to an EGFR inhibitor), tyrosine kinase inhibitor (TKI), and/or a multi-kinase inhibitor (MKI).

In some embodiments, the individual has two or more EGFR inhibitor resistance mutations that increase resistance of the cancer to a first EGFR inhibitor. For example, the individual can have two EGFR inhibitor resistance mutations. In some embodiments, the two mutations occur in the same EGFR protein. In some embodiments, the two mutations occur in separate EGFR proteins. In some embodiments, the individual can have three EGFR inhibitor resistance mutations. In some embodiments, the three mutations occur in the same EGFR protein. In some embodiments, the three mutations occur in separate EGFR proteins. For example, the individual has two or more EGFR inhibitor resistance mutations selected from Del 19/L718Q, Del 19/T790M, Del 19/L844V, Del 19/T790M/L718Q, Del/T790M/C797S, Del 19/T790M/L844V, L858R/L718Q, L858R/L844V, L858R/T790M, L858R/T790M/L718Q, L858R/T790M/C797S, and L858R/T790M/I941R, or any combination thereof; e.g., any two of the aforementioned EGFR inhibitor resistance mutations.

The term “activating mutation” in reference to EGFR describes a mutation in an EGFR gene that results in the expression of an EGFR kinase that has an increased kinase activity, e.g., as compared to a wild type EGFR kinase, e.g., when assayed under identical conditions. For example, an activating mutation can be a mutation in an EGFR gene that results in the expression of an EGFR kinase that has one or more (e.g., two, three, four, five, six, seven, eight, nine, or ten) amino acid substitutions (e.g., any combination of any of the amino acid substitutions described herein) that has increased kinase activity, e.g., as compared to a wild type EGFR kinase, e.g., when assayed under identical conditions. In another example, an activating mutation can be a mutation in an EGFR gene that results in the expression of an EGFR kinase that has one or more (e.g., two, three, four, five, six, seven, eight, nine, or ten) amino acids deleted, e.g., as compared to a wild type EGFR kinase, e.g., when assayed under identical conditions. In another example, an activating mutation can be a mutation in an EGFR gene that results in the expression of an EGFR kinase that has at least one (e.g., at least 2, at least 3, at least 4, at least 5, at least 6, at least 7, at least 8, at least 9, at least 10, at least 12, at least 14, at least 16, at least 18, or at least 20) amino acid inserted as compared to a wild type EGFR kinase, e.g., the exemplary wild type EGFR kinase described herein, e.g., when assayed under identical conditions. Additional examples of activating mutations are known in the art.

The term “wild type” or “wild-type” describes a nucleic acid (e.g., an EGFR gene or an EGFR mRNA) or protein (e.g., an EGFR protein) sequence that is typically found in a subject that does not have a disease or disorder related to the reference nucleic acid or protein.

The term “wild type EGFR” or “wild-type EGFR” describes an EGFR nucleic acid (e.g., an EGFR gene or an EGFR mRNA) or protein (e.g., an EGFR protein) that is found in a subject that does not have an EGFR-associated disease, e.g., an EGFR-associated cancer (and optionally also does not have an increased risk of developing an EGFR-associated disease and/or is not suspected of having an EGFR-associated disease), or is found in a cell or tissue from a subject that does not have an EGFR-associated disease, e.g., an EGFR-associated cancer (and optionally also does not have an increased risk of developing an EGFR-associated disease and/or is not suspected of having an EGFR-associated disease).

Provided herein is a method of treating cancer (e.g., an EGFR-associated cancer) in a subject in need of such treatment, the method comprising administering to the subject a therapeutically effective amount of the compound of Formula (I), or a pharmaceutically acceptable salt thereof, or a pharmaceutical composition thereof. For example, provided herein are methods for treating an EGFR-associated cancer in a subject in need of such treatment, the method comprising a) detecting a dysregulation of an EGFR gene, an EGFR kinase, or the expression or activity or level of any of the same in a sample from the subject; and b) administering a therapeutically effective amount of the compound of Formula (I), or a pharmaceutically acceptable salt thereof.

In some embodiments, the dysregulation of an EGFR gene, an EGFR kinase, or the expression or activity or level of any of the same includes one or more EGFR kinase protein point mutations/insertions. Non-limiting examples of EGFR kinase protein point mutations/insertions/deletions are described in Table 1a and 1b.

In some embodiments, the EGFR kinase protein point mutations/insertions/deletions are selected from the group consisting of G719S, G719C, G719A, L747S, D761Y, T790M, T854A, L858R, L861Q, a deletion in exon 19 (e.g., L747_A750del), and an insertion in exon 20 (e.g., V769_D770insX, D770_N771insX, N771_P772insX, P772_H773insX, or H773_V774insX). In some embodiments, the EGFR kinase protein point mutations/insertions/deletions are selected from the group consisting of L858R, deletions in exon 19 (e.g., L747_A750del), L747S, D761Y, T790M, and T854A. In some embodiments, the EGFR kinase protein insertion is an exon 20 insertion. In some embodiments, the EGFR kinase protein insertion is an exon 20 insertion selected from the group consisting of: V769_D770insX, D770_N771insX, N771_P772insX, P772_H773insX, and H773_V774insX. For example, the EGFR kinase protein insertion is an exon 20 insertion selected from the group consisting of: A767_V769dupASV, V769_D770insASV, D770_N771insNPG, D770_N771insNPY, D770_N771insSVD, D770_N771insGL, N771_H773dupNPH, N771_P772insN, N771_P772insH, N771_P772insV, P772_H773insDNP, P772_H773insPNP, H773_V774insNPH, H773_V774insH, H773_V774insPH, H773_V774insAH, and P772_H773insPNP; or any combination thereof; e.g., any two or more independently selected exon 20 insertions; e.g., any two independently selected exon 20 insertions (e.g., V769 D770insASV and D770_N771insSVD).

In some embodiments of any of the methods or uses described herein, the cancer (e.g., EGFR-associated cancer) is selected from a hematological cancer (e.g., acute lymphocytic cancer, Hodgkin lymphoma, non-Hodgkin lymphoma, and leukemia such as acute-myelogenous leukemia (AML), chronic-myelogenous leukemia (CML), acute-promyelocytic leukemia, and acute lymphocytic leukemia (ALL)), central or peripheral nervous system tissue cancer, an endocrine or neuroendocrine cancer including multiple neuroendocrine type I and type II tumors, Li-Fraumeni tumors, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, oral cancer, oropharyngeal cancer, nasopharyngeal cancer, respiratory cancer, urogenital cancer, cancer of the vulva, colon cancer, esophageal cancer, tracheal cancer, cervical cancer, gastrointestinal carcinoid tumor, hypopharynx cancer, kidney cancer, larynx cancer, liver cancer, lung cancer, malignant mesothelioma, melanoma, multiple myeloma, nasopharynx cancer, ovarian cancer, pancreatic cancer including pancreatic islet cell cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, parathyroid cancer, pituitary tumors, adrenal gland tumors, ureter cancer, biliary cancer, and urinary bladder cancer. In some embodiments, the cancer is selected from the group consisting of: head and neck, ovarian, cervical, bladder and oesophageal cancers, pancreatic, gastrointestinal cancer, gastric, breast, endometrial and colorectal cancers, hepatocellular carcinoma, glioblastoma, bladder, lung cancer, e.g., non-small cell lung cancer (NSCLC), bronchioloalveolar carcinoma. In some embodiments, the cancer is pancreatic cancer, head and neck cancer, melanoma, colon cancer, renal cancer, leukemia, lung cancer, or breast cancer. In some cases, the cancer is melanoma, colon cancer, renal cancer, leukemia, or breast cancer.

In some such embodiments, the compounds provided herein are useful for treating a primary brain tumor or metastatic brain tumor. For example, the compounds can be used in the treatment of one or more of gliomas such as glioblastoma (also known as glioblastoma multiforme), astrocytomas, oligodendrogliomas, ependymomas, and mixed gliomas, meningiomas, medulloblastomas, gangliogliomas, schwannomas (neurilemmomas), and craniopharyngiomas (see, for example, Liu et al. J Exp Clin Cancer Res. 2019 May 23; 38(1):219); and Ding et al. Cancer Res. 2003 Mar. 1; 63(5):1106-13). In some embodiments, the brain tumor is a primary brain tumor. In some embodiments, the brain tumor is a metastatic brain tumor, e.g., a metastatic brain tumor from lung cancer, melanoma, breast cancer, ovarian cancer, colorectal cancer, kidney cancer, bladder cancer, or undifferentiated carcinoma. In some embodiments, the brain tumor is a metastatic brain tumor from lung cancer (e.g., non-small cell lung cancer). In some embodiments, the compounds provided herein exhibit brain and/or central nervous system (CNS) penetrance. In some embodiments, the patient has previously been treated with another anticancer agent, e.g., another EGFR and/or HER2 inhibitor (e.g., a compound that is not a compound of Formula I) or a multi-kinase inhibitor.

In some embodiments, the cancer is a cancer of B cell origin. In some embodiments, the cancer is a lineage dependent cancer. In some embodiments, the cancer is a lineage dependent cancer where EGFR or the dysregulation of an EGFR gene, an EGFR kinase, or expression or activity or level of any of the same, plays a role in the initiation and/or development of the cancer.

In some embodiments, the cancer is an EGFR-associated cancer. Accordingly, also provided herein is a method for treating a subject diagnosed with or identified as having an EGFR-associated cancer, e.g., any of the exemplary EGFR-associated cancers disclosed herein, comprising administering to the subject a therapeutically effective amount of the compound of Formula (I), or a pharmaceutically acceptable salt thereof, or a pharmaceutical composition thereof, as defined herein.

In some embodiments, the dysregulation of an EGFR gene, an EGFR kinase, or expression or activity or level of any of the same, includes one or more deletions (e.g., deletion of an amino acid at position 4), insertions, or point mutation(s) in an EGFR kinase. In some embodiments, dysregulation of an EGFR gene, an EGFR kinase, or expression or activity or level of any of the same, includes at least one deletion, insertion, or point mutation in an EGFR gene that results in the production of an EGFR kinase that has one or more of the amino acid substitutions, insertions, or deletions in Table 1a and Table 1b. In some embodiments, the dysregulation of an EGFR gene, an EGFR kinase, or expression or activity or level of any of the same, includes a deletion of one or more residues from the EGFR kinase, resulting in constitutive activity of the EGFR kinase domain.

In some embodiments, the dysregulation of an EGFR gene, an EGFR kinase, or expression or activity or level of any of the same, includes at least one point mutation in an EGFR gene that results in the production of an EGFR kinase that has one or more amino acid substitutions, insertions, or deletions as compared to the wild type EGFR kinase (see, for example, the point mutations listed in Table 1a and Table 1b). In some embodiments, dysregulation of an EGFR gene, an EGFR kinase, or expression or activity or level of any of the same, includes at least one point mutation in an EGFR gene that results in the production of an EGFR kinase that has one or more of the amino acid substitutions, insertions, or deletions in Table 1a and Table 1b.

In some embodiments, the dysregulation of an EGFR gene, an EGFR kinase, or expression or activity or level of any of the same, includes an insertion of one or more residues in exon 20 of the EGFR gene (e.g., any of the exon 20 insertions described in Table 1a and Table 1b). Exon 20 of EGFR has two major regions, the c-helix (residues 762-766) and the loop following the c-helix (residues 767-774). Studies suggest that for some exon 20 insertions (e.g., insertions after residue 764), a stabilized and ridged active conformation induces resistance to first generation EGFR inhibitors. In some embodiments, the dysregulation of an EGFR gene, an EGFR kinase, or expression or activity or level of any of the same, includes an insertion of one or more residues in exon 20 selected from the group consisting of: V769_D770insX, D770_N771insX, N771_P772insX, P772_H773insX, and H773_V774insX. For example, the EGFR kinase protein insertion is an exon 20 insertion selected from the group consisting of: A767_V769dupASV, V769_D770insASV, D770_N771insNPG, D770_N771insNPY, D770_N771insSVD, D770_N771insGL, N771_H773dupNPH, N771_P772insN, N771_P772insH, N771_P772insV, P772_H773insDNP, P772_H773insPNP, H773_V774insNPH, H773_V774insH, H773_V774insPH, H773_V774insAH, and P772_H773insPNP; or any combination thereof, e.g., any two or more independently selected exon 20 insertions; e.g., any two independently selected exon 20 insertions (e.g., V769 D770insASV and D770_N771insSVD).

TABLE 1a
EGFR Protein Amino Acid Substitutions/Insertions/Deletions^{A}

| Amino Acid | Non-Limiting | Non-limiting Exemplary |
|---|---|---|
| Position(s) | Exemplary Mutations | EGFR-associated Cancer(s) |
| 62 | L62R^{11,B} | |
| 108 | R108K^{11,B} | |
| 216 | A216T^{11,B} | |
| 222 | R222C^{11,B} | |
| 252 | R252C^{11,B} | |
| 289 | A289D, A289T, A289V^{11,B} | |
| 292 | V292L^{11,B} | |
| 304 | H304Y^{11,B} | |
| 306 | S306L^{11,B} | |
| 492 | S492R^{11,B} | |
| 596 | P596L^{11,B} | |
| 598 | G598V^{11,B} | |
| 688 | L688F⁶ | Lung adenocarcinoma⁶ |
| 689 | V689L⁶ | Lung adenocarcinoma⁶ |
| 703 | L703I, L703P^{11,B} | |
| 706 | I706T⁶ | Lung adenocarcinoma⁶ |
| 709 | E709A, E709G, E709K, E709H, | Lung adenocarcinoma^{2,6}, |
| | E709V^{2,6,9} | NSCLC⁹ |
| 709-710 | E709_T710delinsD⁹ | NSCLC⁹ |
| (sometimes | | |
| also called | | |
| exon 18 | | |
| deletion or | | |
| del_18) | | |
| 714 | K714R^{11,B} | |
| 718 | L718Q^{1,11,B} | |
| 719 | G719S, G719C, G719A, G719D^{1,4} | NSCLC³ |
| 719 and 706 | G719A/I706T⁶ | Lung adenocarcinoma⁶ |
| 719 and 709 | G719S/E709K⁶, G719S/E709A^{11,B}, | Lung adenocarcinoma⁶ |
| | G719C/E709A^{11,B} | |
| 719 and exon | G719X/del_18⁹ | NSCLC⁹ |
| 18 deletion | | |
| 720 | S720F^{11,B} | |
| 735 | G735S^{11,B} | |
| 741 | P741L^{11,B} | |
| 744 | I744M^{11,B} | |
| 747 | L747S^{11,B} | |
| 749 | E749Q⁶ | Lung adenocarcinoma⁶ |
| 750 | A750P^{5,10} | NSCLC⁵, lung |
| | | adenocarcinoma¹⁰ |
| 750 and 749 | A750P/E749Q⁶ | |
| 751 | T751I^{11,B} | |
| 752 | S752F¹⁰ | Lung adenocarcinoma¹⁰ |
| 753 | P753S^{11,B} | |
| 765 | V765A³, V765M^{11,B} | NSCLC³ |
| 767 | A767V^{11,B} | |
| 768 | S768I^{2,9} | Lung adenocarcinoma², |
| | | NSCLC⁹ |
| 768 and 719 | S768I/G719A^{11,B}, S768I/G719C^{11,B}, | |
| | S768I/G719S^{11,B}, | |
| 769 | V769M³, V769L^{11,B} | |
| 771 | N771F⁶ | Lung adenocarcinoma⁶ |
| 773 | H773Y⁶, H773L^{11,B}, H773Y^{11,B} | Lung adenocarcinoma⁶ |
| 774 | V774M, V774A^{2,3} | Lung adenocarcinoma², |
| | | NSCLC³ |
| 774 and 773 | V774M/H773L¹⁰ | NSCLC¹⁰ |
| 776 | R776H, R776C⁶, R776G^{11,B} | Lung adenocarcinoma⁶ |
| 776 and 719 | R776H/G719S⁶ | Lung adenocarcinoma⁶ |
| 779 | G779S^{11,B} | |
| 783 | T783A³ | NSCLC³ |
| 784 | S784P³ | |
| 785 | T785I⁶ | Lung adenocarcinoma⁶ |
| 786 | V786M^{11,B} | |
| 790 | T790M³ | NSCLC³ |
| 790 and 719 | T790M/G719A^{11,B} | |
| 790 and 948 | T790M/V948R¹ | |
| 797 and 790 | C797S/T790M^{11,B} | |
| 798 | P798H^{11,B} | |
| 802 | V802I^{11,B} | |
| 813 | Y813H⁶ | Lung adenocarcinoma⁶ |
| 824 | G824S⁶ | Lung adenocarcinoma⁶ |
| 824 and 688 | G824S/L688F⁶ | Lung adenocarcinoma⁶ |
| 831 | R831H, R831L^{11,B} | |
| 833 | L833V^{2,8}, L833F^{11,B} | Lung adenocarcinoma², |
| | | NSCLC⁸ |
| 834 | V834L, V834M^{11,B} | |
| 835 | H835L^{11,B} | |
| 835, 833, and | H835L/L833V/R670W⁸ | NSCLC⁸ |
| 670 | | |
| 838 | L838V^{11,B} | |
| 843 | V843I³ | NSCLC³ |
| 844 | L844V¹ | |
| 845 | V845M⁶ | Lung adenocarcinoma⁶ |
| 848 | P848L^{11,B} | |
| 851 | V851I⁶ | Lung adenocarcinoma⁶ |
| 854 | T854A^{11,B} | |
| 857 | G857R⁶ | Lung adenocarcinoma⁶ |
| 857, 851, 845, | G857R/V851I/V845M/Y813H/T785I⁶ | Lung adenocarcinoma⁶ |
| 813, and 785 | | |
| 858 | L858R¹ | |
| 858 and 108 | L858R/R108K^{11,B} | |
| 858 and 289 | L858R/A289T^{11,B} | |
| 858 and 292 | L858R/V292L^{11,B} | |
| 858 and 306 | L858R/S306L^{11,B} | |
| 858 and 703 | L858R/L703I^{11,B} | |
| 858 and 709 | L858R/E709A^{11,B}, L858R/E709G^{11,B}, | |
| | L858R/E709K^{11,B}, L858R/E709V^{11,B}, | |
| 858 and 714 | L858R/K714R^{11,B} | |
| 858 and 718 | L858R/L718Q¹ | |
| 858 and 720 | L858R/S720F^{11,B} | |
| 858 and 744 | L858R/I744M^{11,B} | |
| 858 and 768 | L858R/S768I^{11,B} | |
| 858 and 769 | L858R/V769L⁶ | Lung adenocarcinoma⁶ |
| 858 and 776 | L858R/R776H⁶, L858R/R776C^{11,B}, | Lung adenocarcinoma⁶ |
| | L858R/R776G^{11,B} | |
| 858 and 790 | L858R/T790M^{11,B} | |
| 858 and 833 | L858R/L833V⁶ | Lung adenocarcinoma⁶ |
| 858 and 838 | L858R/L838V^{11,B} | |
| 858 and 843 | L858R/V843I^{11,B} | |
| 858 and 844 | L858R/L844V¹ | |
| 858 and exon | L858R/del_18⁹ | NSCLC⁹ |
| 18 deletion | | |
| 859 | A859T^{11,B} | |
| 860 | K860R^{11,B} | |
| 861 | L861Q, L861R, L681G^{1,5,6,7,9} | Lung adenocarcinoma⁶, |
| | | NSCLC^{5,9} |
| 861 and 719 | L861Q/G719X¹⁰, L861Q/G719A^{11,B} | Lung adenocarcinoma¹⁰ |
| | L861R/G719A^{11,B} | |
| 861 and 858 | L861Q/L858R^{11,B} | |
| 861, 768, and | L861Q/S768I/G719X¹⁰ | Lung adenocarcinoma¹⁰ |
| 719 | | |
| 864 | A864T^{11,B} | |
| 865 | E865K^{11,B} | |
| 870 | H870R⁶ | Lung adenocarcinoma⁶ |
| 870 and 858 | H870R/L858R | |
| 871 | A871E, A871G^{3,6}, A871T^{11,B} | Lung adenocarcinoma⁶ |
| 871 and 858 | A871G/L858R⁶ | Lung adenocarcinoma⁶ |
| 873 | G873E^{11,B} | |
| 874 | G874S^{11,B} | |
| 941 | I941R¹ | |
| 948 | V948R¹ | |
| 1118 | A1118T^{11,B} | |
| 1153 | S1153I^{11,B} | |
| Exon 19 insertion (sometimes also called ins_19) | | |
| | V738_K739insKIPVAI⁶ | Lung adenocarcinoma⁶ |
| | I744_K745insKIPVAI^{9,10} | NSCLC^{9,10} |
| | K745_E746insTPVAIK^{9,10} | NSCLC^{9,10} |
| | K745_E746insVPVAIK¹⁰ | NSCLC¹⁰ |
| | K745_E746insIPVAIK^{9,10} | NSCLC^{9,10} |
| Exon 19 deletion (sometimes also called del_19)^{1,2} | | |
| | E746_A750del¹ | |
| | E746_A750delinsP¹ | |
| | E746_A750delinsIP^{11,B} | |
| | E746_A750del/T790M^{11,B} | |
| | E746_A750del/A1118T^{11,B} | |
| | E746_T751delinsV^{11,B} | |
| | E746_S752delinsV² | Lung adenocarcinoma² |
| | E746_S752delinsV/A216T^{11,B} | |
| | L747_E749del⁵ | NSCLC⁵ |
| | L747_A750del³ | NSCLC³ |
| | L747_A750delinsP² | Lung adenocarcinoma² |
| | L747_T751del² | Lung adenocarcinoma² |
| | L747_T751delinsA¹⁰ | NSCLC¹⁰ |
| | L747_T751delinsP¹⁰ | NSCLC¹⁰ |
| | L747_T751delinsS^{11,B} | |
| | L747_T751delinsQ² | Lung adenocarcinoma² |
| | L747_S752del² | Lung adenocarcinoma² |
| | L747_P753del¹⁰ | NSCLC¹⁰ |
| | L747_P753delinsS² | Lung adenocarcinoma² |
| | L747_P753delinsQ^{11,B} | |
| | L747_P753delinsVS¹⁰ | NSCLC¹⁰ |
| | T751_I759delinsN^{11,B} | |
| | S752_I759del^{2,10} | Lung adenocarcinoma², |
| | | NSCLC¹⁰ |
| Exon 19 | Del_19 and I706T⁶ | Lung adenocarcinoma⁶ |
| deletion and | | |
| 706 | | |
| Exon 19 | Del_19 and L718Q¹ | |
| deletion and | | |
| 718 | | |
| Exon 19 | Del_19 and L844V¹ | |
| deletion and | | |
| 844 | | |
| Exon 19 | Del_19 and L858R⁶ | Lung adenocarcinoma⁶ |
| deletion and | | |
| 858 | | |
| Exon 19 | Del_19 and del_18⁹ | NSCLC⁹ |
| deletion and | | |
| Exon 18 | | |
| deletion | | |
| Exon 20 insertion (sometimes also called ins_20)¹ | | |
| | D761_E762insX⁵, e.g., | NSCLC⁵ |
| | D761_E762insEAFQ¹⁰ | |
| | A763_Y764insX⁵, e.g., | NSCLC^{5,9,10} |
| | A763_Y764insFQEA^{5,9,10} | |
| | Y764_V765insX⁵, e.g., | NSCLC⁵ |
| | V764_V765insHH¹³ | |
| | V765_M766insX⁵ | NSCLC⁵ |
| | M766_A767insASV⁶ | Lung adenocarcinoma⁶ |
| | A767_S768insX⁵; A767insASV¹⁴ | NSCLC⁵ |
| | A767_V769dupASV⁵ | NSCLC⁵ |
| | S768_V769insX¹²; S768dupSVD¹⁴ | NSCLC |
| | V769_D770insX⁵, e.g., | NSCLC^{5,8,10} |
| | V769_D770insASV^{8,9,10} | |
| | D770delinsGY^{5,10} | NSCLC^{5,10} |
| | D770_N771insX⁵, e.g., | NSCLC^{3,5,10} |
| | D770_N771insNPG^{2,5,9}, | |
| | D770_N771insG + N771T⁶, | |
| | D770_N771insNPY⁶, | |
| | D770_N771insSVD^{9,10}, | |
| | D770_N771insGL¹³ | |
| | D770_N771insX and amino acid position | Lung adenocarcinoma⁶ |
| | 773, e.g., D770_N771insNPY/H773Y⁶ | |
| | N771_P772insX⁵, e.g., N771_P772insN¹⁰, | NSCLC^{5,10} |
| | N771_P772insH¹³, N771_P772insV¹³ | |
| | N771_H773dupNPH⁵ | NSCLC⁵ |
| | N771delinsGY¹³; N771del insFH¹⁴ | |
| | P772_H773insX⁵, e.g., | NSCLC^{5,9} |
| | P772_H773insDNP⁹, P772_H773insPNP⁹ | |
| | H773_V774insX⁵, e.g., | NSCLC^{5,9,10} |
| | H773_V774insNPH⁹, H773_V774insH¹⁰, | |
| | H773_V774insPH¹⁰, H773_V774insAH¹⁰, | |
| | P772_H773insPNP¹³ | |
| | H773_dupH¹³ | |
| | V774_C775insX⁵, e.g., | NSCLC⁵ |
| | V774_C775insHV¹⁰ | |
| | A775_G776insX, e.g., | |
| | A775_G776insYVMA¹³ | |
| Kinase domain | Duplication of exons 18-25, 18-26, 14-26, | NSCLC⁹ |
| duplication | or 17-25⁹ | |
| (KDD) | | |

^{A}The EGFR mutations shown may be activating mutations and/or confer increased resistance of EGFR to an EGFR inhibitor and/or a multi-kinase inhibitor (MKI), e.g., as compared to a wild type EGFR
^{B}Potentially oncogenic variant. See, e.g., Kohsaka, Shinji, et al. Science translational medicine 9.416 (2017): eaan6566.
¹PCT Patent Application Publication No. WO2019/246541.
²Grosse A, Grosse C, Rechsteiner M, Soltermann A. Diagn Pathol. 2019; 14(1): 18. Published 2019 Feb. 11. doi: 10.1186/s13000-019-0789-1.
³Stewart E L, Tan S Z, Liu G, Tsao M S. Transl Lung Cancer Res. 2015; 4(1): 67-81. doi: 10.3978/j.issn.2218-6751.2014.11.06.
⁴Pines, Gur, Wolfgang J. Köstler, and Yosef Yarden. FEBS letters 584.12 (2010): 2699-2706.
⁵Yasuda, Hiroyuki, Susumu Kobayashi, and Daniel B. Costa. The Lancet Oncology 13.1 (2012): e23-e31.
⁶Kim E Y, Cho E N, Park H S, et al. Cancer Biol Ther. 2016; 17(3): 237-245. doi: 10.1080/15384047.2016.1139235.
⁷Shah, Riyaz, and Jason F. Lester. Clinical Lung Cancer (2019).
⁸Aran, Veronica, and Jasminka Omerovic. International journal of molecular sciences 20.22 (2019): 5701. doi: 10.3390/ijms20225701.
⁹Beau-Faller, Michele, et al. (2012): 10507-10507. doi: 10.1016/j.semcancer.2019.09.015.
¹⁰Masood, Ashiq, Rama Krishna Kancha, and Janakiraman Subramanian. Seminars in oncology. W B Saunders, 2019. doi: 10.1053/j.seminoncol.2019.08.004.
¹¹Kohsaka, Shinji, et al. Science translational medicine 9.416 (2017): eaan6566.
¹²Vyse and Huang et al. Signal Transduct Target Ther. 2019 Mar. 8; 4: 5. doi: 10.1038/s41392-019-0038-9.
¹³PCT Patent Application Publication No. WO2019/046775.
¹⁴PCT Patent Application Publication No. WO 2018/094225.

TABLE 1b
EGFR Protein Amino Acid Substitutions/Insertions/Deletions^{A}

| Amino Acid | Non-Limiting | Non-limiting Exemplary |
|---|---|---|
| Position(s) | Exemplary Mutations | EGFR-associated Cancer(s) |
| 62 | L62R^{11,B} | |
| 108 | R108K^{11,B} | |
| 216 | A216T^{11,B} | |
| 222 | R222C^{11,B} | |
| 252 | R252C^{11,B} | |
| 289 | A289D, A289T, A289V^{11,B} | |
| 292 | V292L^{11,B} | |
| 304 | H304Y^{11,B} | |
| 306 | S306L^{11,B} | |
| 492 | S492R^{11,B} | |
| 596 | P596L^{11,B} | |
| 598 | GS98V^{11,B} | |
| 688 | L688F⁶ | Lung adenocarcinoma⁶ |
| 689 | V689L⁶ | Lung adenocarcinoma⁶ |
| 703 | L703I, L703P^{11,B} | |
| 706 | I706T⁶ | Lung adenocarcinoma⁶ |
| 709 | E709A, E709G, E709K, E709H, | Lung adenocarcinoma^{2,6}, |
| | E709V^{2,6,9} | NSCLC⁹ |
| 709-710 | E709_T710delinsD⁹ | NSCLC⁹ |
| (sometimes | | |
| also called | | |
| exon 18 | | |
| deletion or | | |
| del_18) | | |
| 714 | K714R^{11,B} | |
| 718 | L718Q^{1,11,B} | |
| 719 | G719S, G719C, G719A, G719D^{1,4} | NSCLC³ |
| 719 and 706 | G719A/I706T⁶ | Lung adenocarcinoma⁶ |
| 719 and 709 | G719S/E709K⁶, G719S/E709A^{11,B}, | Lung adenocarcinoma⁶ |
| | G719C/E709A^{11,B} | |
| 719 and exon | G719X/del_18⁹ | NSCLC⁹ |
| 18 deletion | | |
| 720 | S720F^{11,B} | |
| 735 | G735S^{11,B} | |
| 741 | P741L^{11,B} | |
| 744 | I744M^{11,B} | |
| 747 | L747S^{11,B} | |
| 749 | E749Q⁶ | Lung adenocarcinoma⁶ |
| 750 | A750P^{5,10} | NSCLC⁵, lung |
| | | adenocarcinoma¹⁰ |
| 750 and 749 | A750P/E749Q⁶ | |
| 751 | T751I^{11,B} | |
| 752 | S752F¹⁰ | Lung adenocarcinoma¹⁰ |
| 753 | P753S^{11,B} | |
| 765 | V765A³, V765M^{11,B} | NSCLC³ |
| 767 | A767V^{11,B} | |
| 768 | S768I^{2,9} | Lung adenocarcinoma², |
| | | NSCLC⁹ |
| 768 and 719 | S768I/G719A^{11,B}, S7681/G719C^{11,B}, | |
| | S768I/G7198^{11,B}, | |
| 769 | V769M³, V769L^{11,B} | |
| 771 | N771F⁶ | Lung adenocarcinoma⁶ |
| 773 | H773Y⁶, H773L^{11,B}, H773Y^{11,B} | Lung adenocarcinoma⁶ |
| 774 | V774M, V774A^{2,3} | Lung adenocarcinoma², |
| | | NSCLC³ |
| 774 and 773 | V774M/H773L¹⁰ | NSCLC¹⁰ |
| 776 | R776H, R776C⁶, R776G^{11,B} | Lung adenocarcinoma⁶ |
| 776 and 719 | R776H/G719S⁶ | Lung adenocarcinoma⁶ |
| 779 | G779S^{11,B} | |
| 783 | T783A³ | NSCLC³ |
| 784 | S784P³ | |
| 785 | T785I⁶ | Lung adenocarcinoma⁶ |
| 786 | V786M^{11,B} | |
| 790 | T790M³ | NSCLC³ |
| 790 and 719 | T790M/G719A^{11,B} | |
| 790 and 948 | T790M/V948R¹ | |
| 797 and 790 | C797S/T790M^{11,B} | |
| 798 | P798H^{11,B} | |
| 802 | V802I^{11,B} | |
| 813 | Y813H⁶ | Lung adenocarcinoma⁶ |
| 824 | G824S⁶ | Lung adenocarcinoma⁶ |
| 824 and 688 | G824S/L688F⁶ | Lung adenocarcinoma⁶ |
| 831 | R831H, R831L^{11,B} | |
| 833 | L833V^{2,8}, L833F^{11,B} | Lung adenocarcinoma², |
| | | NSCLC⁸ |
| 834 | V834L, V834M^{11,B} | |
| 835 | H835L^{11,B} | |
| 835, 833, and | H835L/L833V/R670W⁸ | NSCLC⁸ |
| 670 | | |
| 838 | L838V^{11,B} | |
| 843 | V843I³ | NSCLC³ |
| 844 | L844V¹ | |
| 845 | V845M⁶ | Lung adenocarcinoma⁶ |
| 848 | P848L^{11,B} | |
| 851 | V851I⁶ | Lung adenocarcinoma⁶ |
| 854 | T854A^{11,B} | |
| 857 | G857R⁶ | Lung adenocarcinoma⁶ |
| 857, 851, 845, | G857R/V851I/V845M/Y813H/T785I⁶ | Lung adenocarcinoma⁶ |
| 813, and 785 | | |
| 858 | L858R¹ | |
| 858 and 108 | L858R/R108K^{11,B} | |
| 858 and 289 | L858R/A289T^{11,B} | |
| 858 and 292 | L858R/V292L^{11,B} | |
| 858 and 306 | L858R/S306L^{11,B} | |
| 858 and 703 | L858R/L703I^{11,B} | |
| 858 and 709 | L858R/E709A^{11,B}, L858R/E709G^{11,B}, | |
| | L858R/E709K^{11,B}, L858R/E709V^{11,B}, | |
| 858 and 714 | L858R/K714R^{11,B} | |
| 858 and 718 | L858R/L718Q¹ | |
| 858 and 720 | L858R/S720F^{11,B} | |
| 858 and 744 | L858R/I744M^{11,B} | |
| 858 and 768 | L858R/S768I^{11,B} | |
| 858 and 769 | L858R/V769L⁶ | Lung adenocarcinoma⁶ |
| 858 and 776 | L858R/R776H⁶, L858R/R776C^{11,B}, | Lung adenocarcinoma⁶ |
| | L858R/R776G^{11,B} | |
| 858 and 790 | L858R/T790M^{11,B} | |
| 858 and 833 | L858R/L833V⁶ | Lung adenocarcinoma⁶ |
| 858 and 838 | L858R/L838V^{11,B} | |
| 858 and 843 | L858R/V843I^{11,B} | |
| 858 and 844 | L858R/L844V¹ | |
| 858 and exon | L858R/del_18⁹ | NSCLC⁹ |
| 18 deletion | | |
| 859 | A859T^{11,B} | |
| 860 | K860R^{11,B} | |
| 861 | L861Q, L861R, L681G^{1,5,6,7,9} | Lung adenocarcinoma⁶, |
| | | NSCLC^{5,9} |
| 861 and 719 | L861Q/G719X¹⁰, L861Q/G719A^{11,B}, | Lung adenocarcinoma¹⁰ |
| | L861R/G719A^{11,B} | |
| 861 and 858 | L861Q/L858R^{11,B} | |
| 861, 768, and | L861Q/S7681/G719X¹⁰ | Lung adenocarcinoma¹⁰ |
| 719 | | |
| 864 | A864T^{11,B} | |
| 865 | E865K^{11,B} | |
| 870 | H870R⁶ | Lung adenocarcinoma⁶ |
| 870 and 858 | H870R/L858R | |
| 871 | A871E, A871G^{3,6}, A871T^{11,B} | Lung adenocarcinoma⁶ |
| 871 and 858 | A871G/L858R⁶ | Lung adenocarcinoma⁶ |
| 873 | G873E^{11,B} | |
| 874 | G874S^{11,B} | |
| 941 | I941R¹ | |
| 948 | V948R¹ | |
| 1118 | A1118T^{11,B} | |
| 1153 | S1153I^{11,B} | |
| Exon 19 insertion (sometimes also called ins_19) | | |
| | V738_K739insKIPVAI⁶ | Lung adenocarcinoma⁶ |
| | I744_K745insKIPVAI^{9,10} | NSCLC^{9,10} |
| | K745_E746insTPVAIK^{9,10} | NSCLC^{9,10} |
| | K745_E746insVPVAIK¹⁰ | NSCLC¹⁰ |
| | K745_E746insIPVAIK^{9,10} | NSCLC^{9,10} |
| Exon 19 deletion (sometimes also called del_19)^{1,2} | | |
| | E746_A750del¹ | |
| | E746_A750delinsP¹ | |
| | E746_A750delinsIP^{11,B} | |
| | E746_A750del/T790M^{11,B} | |
| | E746_A750del/A1118T^{11,B} | |
| | E746_T751delinsV^{11,B} | |
| | E746_S752delinsV² | Lung adenocarcinoma² |
| | E746_S752delinsV/A216T^{11,B} | |
| | L747_E749del⁵ | NSCLC⁵ |
| | L747_A750del³ | NSCLC³ |
| | L747_A750delinsP² | Lung adenocarcinoma² |
| | L747_T751del² | Lung adenocarcinoma² |
| | L747_T751delinsA¹⁰ | NSCLC¹⁰ |
| | L747_T751delinsP¹⁰ | NSCLC¹⁰ |
| | L747_T751delinsS^{11,B} | |
| | L747_T751delinsQ² | Lung adenocarcinoma² |
| | L747_S752del² | Lung adenocarcinoma² |
| | L747_P753del¹⁰ | NSCLC¹⁰ |
| | L747_P753delinsS² | Lung adenocarcinoma² |
| | L747_P753delinsQ^{11,B} | |
| | L747_P753delinsVS¹⁰ | NSCLC¹⁰ |
| | T751_I759delinsN^{11,B} | |
| | S752_I759del^{2,10} | Lung adenocarcinoma², |
| | | NSCLC¹⁰ |
| Exon 19 | Del_19 and I706T⁶ | Lung adenocarcinoma⁶ |
| deletion and | | |
| 706 | | |
| Exon 19 | Del_19 and L718Q¹ | |
| deletion and | | |
| 718 | | |
| Exon 19 | Del_19 and L844V¹ | |
| deletion and | | |
| 844 | | |
| Exon 19 | Del_19 and L858R⁶ | Lung adenocarcinoma⁶ |
| deletion and | | |
| 858 | | |
| Exon 19 | Del_19 and del_18⁹ | NSCLC⁹ |
| deletion and | | |
| Exon 18 | | |
| deletion | | |
| Exon 20 insertion (sometimes also called ins_20)¹ | | |
| | D761_E762insX⁵, e.g., | NSCLC⁵ |
| | D761_E762insEAFQ¹⁰ | |
| | A763_Y764insX⁵, e.g., | NSCLC^{5,9,10} |
| | A763_Y764insFQEA^{5,9,10} | |
| | Y764_V765insX⁵, e.g., | NSCLC⁵ |
| | V764_V765insHH¹³ | |
| | V765_M766insX⁵ | NSCLC⁵ |
| | M766_A767insASV⁶ | Lung adenocarcinoma⁶ |
| | M766delinsMASVx2¹⁵ | Pediatric bithalamic |
| | | glioma¹⁵ |
| | A767_S768insX⁵; A767insASV¹⁴ | NSCLC⁵ |
| | A767_V769dupASV⁵ | NSCLC⁵ |
| | A767delinsASVDx3; | Pediatric bithalamic |
| | A767delinsASVG¹⁵ | glioma¹⁵ |
| | S768_V769insX¹²; S768dupSVD^{14,16} | NSCLC^{12,14}; Sinonasal |
| | | squamous cell |
| | | carcinoma¹⁶ |
| | V769_D770insX⁵, e.g., | NSCLC^{5,8,10} |
| | V769_D770insASV^{8,9,10} | |
| | D770delinsGY^{5,10}; D770delinsDN; | NSCLC^{5,10}; Pediatric |
| | D770delinsDNPH¹⁵ | bithalamic glioma¹⁵ |
| | D770_N771insX^{5,16}, e.g., | NSCLC^{3,5,10}; Sinonasal |
| | D770_N771insNPG^{2,5,9}, | squamous cell |
| | D770_N771insG + N771T⁶, | carcinoma¹⁶ |
| | D770_N771insNPY⁶, | |
| | D770_N771insSVD^{9,10}, | |
| | D770_N771insGL¹³ | |
| | D770_N771insX and amino acid position | Lung adenocarcinoma⁶ |
| | 773, e.g., D770_N771insNPY/H773Y⁶ | |
| | N771_P772insX^{5,16}, e.g., N771_P772insN¹⁰, | NSCLC^{5,10} Sinonasal |
| | N771_P772insH¹³, N771_P772insV¹³ | squamous cell |
| | | carcinoma¹⁶ |
| | N771_H773dupNPH⁵ | NSCLC⁵ |
| | N771delinsGY¹³; N771del insFH¹⁴; | NSCLC^{13,14}; Pediatric |
| | N771delinsNPH¹⁵ | bithalamic glioma¹⁵ |
| | N771_H773dup¹⁶ | Sinonasal squamous |
| | | cell carcinoma¹⁶ |
| | P772_H773insX⁵, e.g., | NSCLC^{5,9} |
| | P772_H773insDNP⁹, P772_H773insPNP⁹ | |
| | H773_V774insX⁵, e.g., | NSCLC^{5,9,10} |
| | H773_V774insNPH⁹, H773_V774insH¹⁰, | |
| | H773_V774insPH¹⁰, H773_V774insAH¹⁰, | |
| | P772_H773insPNP¹³ | |
| | H773_dupH¹³ | |
| | V774_C775insX⁵, e.g., | NSCLC⁵ |
| | V774_C775insHV¹⁰ | |
| | A775_G776insX, e.g., | |
| | A775_G776insYVMA¹³ | |
| Kinase domain | Duplication of exons 18-25, 18-26, 14-26, | NSCLC⁹ |
| duplication | or 17-25⁹ | |
| (KDD) | | |

^{A}The EGFR mutations shown may be activating mutations and/or confer increased resistance of EGFR to an EGFR inhibitor and/or a multi-kinase inhibitor (MKI), e.g., as compared to a wild type EGFR
^{B}Potentially oncogenic variant. See, e.g., Kohsaka, Shinji, et al. Science translational medicine 9.416 (2017): eaan6566.
¹PCT Patent Application Publication No. WO2019/246541.
²Grosse A, Grosse C, Rechsteiner M, Soltermann A. Diagn Pathol. 2019; 14(1): 18. Published 2019 Feb. 11. doi: 10.1186/s13000-019-0789-1.
³Stewart E L, Tan S Z, Liu G, Tsao M S. Transl Lung Cancer Res. 2015; 4(1): 67-81. doi: 10.3978/j.issn.2218-6751.2014.11.06.
⁴Pines, Gur, Wolfgang J. Köstler, and Yosef Yarden. FEBS letters 584.12 (2010): 2699-2706.
⁵Yasuda, Hiroyuki, Susumu Kobayashi, and Daniel B. Costa. The Lancet Oncology 13.1 (2012): e23-e31.
⁶Kim E Y, Cho E N, Park H S, et al. Cancer Biol Ther. 2016; 17(3): 237-245. doi: 10.1080/15384047.2016.1139235.
⁷Shah, Riyaz, and Jason F. Lester. Clinical Lung Cancer (2019).
⁸Aran, Veronica, and Jasminka Omerovic. International journal of molecular sciences 20.22 (2019): 5701. doi: 10.3390/ijms20225701.
⁹Beau-Faller, Michele, et al. (2012): 10507-10507. doi: 10.1016/j.semcancer.2019.09.015.
¹⁰Masood, Ashiq, Rama Krishna Kancha, and Janakiraman Subramanian. Seminars in oncology. W B Saunders, 2019. doi: 10.1053/j.seminoncol.2019.08.004.
¹¹Kohsaka, Shinji, et al. Science translational medicine 9.416 (2017): eaan6566.
¹²Vyse and Huang et al. Signal Transduct Target Ther. 2019 Mar. 8; 4: 5. doi: 10.1038/s41392-019-0038-9.
¹³PCT Patent Application Publication No. WO2019/046775.
¹⁴PCT Patent Application Publication No. WO 2018/094225.
¹⁵Mondal, Gourish, et al. Acta Neuropathol. 2020; 139(6): 1071-1088
¹⁶Udager, Aaron M., et al. Cancer Res, 2015; 75(13): 2600-2606

In some embodiments, the dysregulation of an EGFR gene, an EGFR kinase, or expression or activity or level of any of the same, includes a splice variation in an EGFR mRNA which results in an expressed protein that is an alternatively spliced variant of EGFR having at least one residue deleted (as compared to the wild type EGFR kinase) resulting in a constitutive activity of an EGFR kinase domain.

In some embodiments, the dysregulation of an EGFR gene, an EGFR kinase, or expression or activity or level of any of the same, includes at least one point mutation in an EGFR gene that results in the production of an EGFR kinase that has one or more amino acid substitutions or insertions or deletions in an EGFR gene that results in the production of an EGFR kinase that has one or more amino acids inserted or removed, as compared to the wild type EGFR kinase. In some cases, the resulting EGFR kinase is more resistant to inhibition (e.g., inhibition of its signaling activity) by one or more first EGFR inhibitors, as compared to a wild type EGFR kinase or an EGFR kinase not including the same mutation. Such mutations, optionally, do not decrease the sensitivity of the cancer cell or tumor having the EGFR kinase to treatment with the compound of Formula (I), or a pharmaceutically acceptable salt thereof, (e.g., as compared to a cancer cell or a tumor that does not include the particular EGFR inhibitor resistance mutation).

In other embodiments, the dysregulation of an EGFR gene, an EGFR kinase, or expression or activity or level of any of the same, includes at least one point mutation in an EGFR gene that results in the production of an EGFR kinase that has one or more amino acid substitutions as compared to the wild type EGFR kinase, and which has increased resistance to the compound of Formula (I), or a pharmaceutically acceptable salt thereof, as compared to a wild type EGFR kinase or an EGFR kinase not including the same mutation. In such embodiments, an EGFR inhibitor resistance mutation can result in an EGFR kinase that has one or more of an increased Vₘₐₓ, a decreased Kₘ, and a decreased K_{D} in the presence of the compound of Formula (I), or a pharmaceutically acceptable salt thereof, as compared to a wild type EGFR kinase or an EGFR kinase not having the same mutation in the presence of the compound of Formula (I), or a pharmaceutically acceptable salt thereof.

Exemplary Sequence of Mature Human EGFR Protein
(UniProtKB entry P00533)
(SEQ ID NO: 1)
MRPSGTAGAA LLALLAALCP ASRALEEKKV CQGTSNKLTQ
LGTFEDHFLS LQRMFNNCEV VLGNLEITYV QRNYDLSFLK
TIQEVAGYVL IALNTVERIP LENLQIIRGN MYYENSYALA
VLSNYDANKT GLKELPMRNL QEILHGAVRF SNNPALCNVE
SIQWRDIVSS DFLSNMSMDF QNHLGSCQKC DPSCPNGSCW
GAGEENCQKL TKIICAQQCS GRCRGKSPSD CCHNQCAAGC
TGPRESDCLV CRKFRDEATC KDTCPPLMLY NPTTYQMDVN
PEGKYSFGAT CVKKCPRNYV VTDHGSCVRA CGADSYEMEE
DGVRKCKKCE GPCRKVCNGI GIGEFKDSLS INATNIKHFK
NCTSISGDLH ILPVAFRGDS FTHTPPLDPQ ELDILKTVKE
ITGFLLIQAW PENRTDLHAF ENLEIIRGRT KQHGQFSLAV
VSLNITSLGL RSLKEISDGD VIISGNKNLC YANTINWKKL
FGTSGQKTKI ISNRGENSCK ATGQVCHALC SPEGCWGPEP
RDCVSCRNVS RGRECVDKCN LLEGEPREFV ENSECIQCHP
ECLPQAMNIT CTGRGPDNCI QCAHYIDGPH CVKTCPAGVM
GENNTLVWKY ADAGHVCHLC HPNCTYGCTG PGLEGCPTNG
PKIPSIATGM VGALLLLLVV ALGIGLFMRR RHIVRKRTLR
RLLQERELVE PLTPSGEAPN QALLRILKET EFKKIKVLGS
GAFGTVYKGL WIPEGEKVKI PVAIKELREA TSPKANKEIL
DEAYVMASVD NPHVCRLLGI CLTSTVQLIT QLMPFGCLLD
YVREHKDNIG SQYLLNWCVQ IAKGMNYLED RRLVHRDLAA
RNVLVKTPQH VKITDFGLAK LLGAEEKEYH AEGGKVPIKW
MALESILHRI YTHQSDVWSY GVTVWELMTF GSKPYDGIPA
SEISSILEKG ERLPQPPICT IDVYMIMVKC WMIDADSRPK
FRELIIEFSK MARDPQRYLV IQGDERMHLP SPTDSNFYRA
LMDEEDMDDV VDADEYLIPQ QGFFSSPSTS RTPLLSSLSA
TSNNSTVACI DRNGLQSCPI KEDSFLQRYS SDPTGALTED
SIDDTFLPVP EYINQSVPKR PAGSVQNPVY HNQPLNPAPS
RDPHYQDPHS TAVGNPEYLN TVQPTCVNST FDSPAHWAQK
GSHQISLDNP DYQQDFFPKE AKPNGIFKGS TAENAEYLRV
APQSSEFIGA

In some embodiments, dysregulation of an EGFR gene, an EGFR kinase, or expression or activity or level of any of the same, includes at least one EGFR inhibitor resistance mutation in an EGFR gene that results in the production of an EGFR kinase that has one or more of the amino acid substitutions, insertions, or deletions as described in Table 2a and Table 2b. In some embodiments, the compound of Formula (I) and pharmaceutically acceptable salts and solvates thereof, are useful in treating subjects that develop cancers with EGFR inhibitor resistance mutations (e.g., that result in an increased resistance to a first EGFR inhibitor, e.g., a substitution at amino acid position 718, 747, 761, 790, 797, or 854 (e.g., L718Q, L747S, D761Y, T790M, C797S, T854A), and/or one or more EGFR inhibitor resistance mutations listed in Table 2a and Table 2b) by either dosing in combination or as a subsequent or additional (e.g., follow-up) therapy to existing drug treatments (e.g., other inhibitors of EGFR; e.g., first and/or second EGFR inhibitors).

TABLE 2a
EGFR Protein Amino Acid Resistance Mutations

| Amino Acid | Non-Limiting | Non-limiting Exemplary |
|---|---|---|
| Position(s) | Exemplary Mutations | EGFR-associated Cancer(s) |
| 747 | L747S^{2,4,6}, L747P⁶ | NSCLC² |
| 761 | D761Y^{2,4} | NSCLC² |
| 769 | V769M² | NSCLC² |
| 790 | T790M^{1,4} | NSCLC² |
| 792 | L792H⁶ | NSCLC⁶ |
| 796 | G796R⁶ | NSCLC⁶ |
| 797 | C7978⁵ | NSCLC⁵ |
| 797 and 790 | C797S/T790M⁵ | NSCLC⁵ |
| 843 | V843I^{2,5} | NSCLC² |
| 854 | T854A^{2,4} | NSCLC² |
| 858 and 747 | L858R/L747S⁶ | NSCLC⁶ |
| 858 and 790 | L858R/T790M¹ | |
| 858, 797, and | L858R/C797S/T790M¹ | |
| 790 | | |
| 871 | A871E² | NSCLC² |
| 941, 858, and | I941R/L858R/T790M¹ | |
| 790 | | |
| Exon 19 | Del_19 and T790M¹ | |
| deletion and | | |
| 790 | | |
| Exon 19 | Del_19 and T790M/L844V¹ | |
| deletion, 844, | | |
| and 790 | | |
| Exon 19 | Del_19 and C797S/T790M¹ | |
| deletion, 797, | | |
| and 790 | | |
| Exon 20 insertion (also called ins_20)^{2,3} | | |
| | A767_V769dupASV⁵ | NSCLC⁵ |
| | D770_N771insX³, e.g., | NSCLC^{2,3} |
| | D770_N771insNPG^{2,3,7} | |
| | N771_H771dupNPH³ | NSCLC³ |
| | P772_H773insX³ e.g., | NSCLC^{3,7} |
| | P772_H773insDNP⁷ | |
| | H773_V774insNPH⁷ | NSCLC⁷ |

¹PCT Patent Application Publication No. WO2019/246541
²Stewart E L, Tan S Z, Liu G, Tsao M S. Transl Lung Cancer Res. 2015; 4(1): 67-81. doi: 10.3978/j.issn.2218-6751.2014.11.06
³Yasuda, Hiroyuki, Susumu Kobayashi, and Daniel B. Costa. The Lancet Oncology 13.1 (2012): e23-e31.
⁴Kim E Y, Cho E N, Park H S, et al. Cancer Biol Ther. 2016; 17(3): 237-245. doi: 10.1080/15384047.2016.1139235
⁵Shah, Riyaz, and Jason F. Lester. Clinical Lung Cancer (2019).
⁶Aran, Veronica, and Jasminka Omerovic. International journal of molecular sciences 20.22 (2019): 5701. doi: 10.3390/ijms20225701.
⁷Beau-Faller, Michele, et al. (2012): 10507-10507. doi: 10.1016/j.semcancer.2019.09.015
⁸Masood, Ashiq, Rama Krishna Kancha, and Janakiraman Subramanian. Seminars in oncology. W B Saunders, 2019. doi: 10.1053/j.seminoncol.2019.08.004

TABLE 2b
EGFR Protein Amino Acid Resistance Mutations

| Amino Acid | Non-Limiting | Non-limiting Exemplary |
|---|---|---|
| Position(s) | Exemplary Mutations | EGFR-associated Cancer(s) |
| 747 | L747S^{2,4,6}, L747P⁶ | NSCLC² |
| 761 | D761Y^{2,4} | NSCLC² |
| 769 | V769M² | NSCLC² |
| 790 | T790M^{1,4} | NSCLC² |
| 792 | L792H⁶ | NSCLC⁶ |
| 796 | G796R⁶ | NSCLC⁶ |
| 797 | C797S⁵ | NSCLC⁵ |
| 797 and 790 | C797S/T790M⁵ | NSCLC⁵ |
| 843 | V843I^{2,5} | NSCLC² |
| 854 | T854A^{2,4} | NSCLC² |
| 858 and 747 | L858R/L747S⁶ | NSCLC⁶ |
| 858 and 790 | L858R/T790M¹ | |
| 858 and 797 | L858R/C797S (or C797G)⁹ | NSCLC⁹ |
| 858, 797, and | L858R/C797S/T790M¹ | |
| 790 | | |
| 871 | A871E² | NSCLC² |
| 941, 858, and | I941R/L858R/T790M¹ | |
| 790 | | |
| Exon 19 | Del_19 and T790M¹ | |
| deletion and | | |
| 790 | | |
| Exon 19 | Del_19 and C797S (or C797G)⁹ | NSCLC⁹ |
| deletion and | | |
| 797 | | |
| Exon 19 | Del_19 and T790M/L844V¹ | |
| deletion, 844, | | |
| and 790 | | |
| Exon 19 | Del_19 and C797S/T790M¹ | |
| deletion, 797, | | |
| and 790 | | |
| Exon 20 insertion (also called ins_20)^{2,3} | | |
| | A767_V769dupASV⁵ | NSCLC⁵ |
| | D770_N771insX³, e.g., | NSCLC^{2,3} |
| | D770_N771insNPG^{2,3,7} | |
| | N771_H771dupNPH³ | NSCLC³ |
| | P772_H773insX³ e.g., | NSCLC^{3,7} |
| | P772_H773insDNP⁷ | |
| | H773_V774insNPH⁷ | NSCLC⁷ |

¹PCT Patent Application Publication No. WO2019/246541
²Stewart E L, Tan S Z, Liu G, Tsao M S. Transl Lung Cancer Res. 2015; 4(1): 67-81. doi: 10.3978/j.issn.2218-6751.2014.11.06
³Yasuda, Hiroyuki, Susumu Kobayashi, and Daniel B. Costa. The Lancet Oncology 13.1 (2012): e23-e31.
⁴Kim E Y, Cho E N, Park H S, et al. Cancer Biol Ther. 2016; 17(3): 237-245. doi: 10.1080/15384047.2016.1139235
⁵Shah, Riyaz, and Jason F. Lester. Clinical Lung Cancer (2019).
⁶Aran, Veronica, and Jasminka Omerovic. International journal of molecular sciences 20.22 (2019): 5701. doi: 10.3390/ijms20225701.
⁷Beau-Faller, Michele, et al. (2012): 10507-10507. doi: 10.1016/j.semcancer.2019.09.015
⁸Masood, Ashiq, Rama Krishna Kancha, and Janakiraman Subramanian. Seminars in oncology. W B Saunders, 2019. doi: 10.1053/j.seminoncol.2019.08.004
⁹Papadimitrakopoulou, V. A., et al. Annals of Oncology 2018; 29 Supplement 8 VIII741

In some embodiments, the EGFR Protein Amino Acid Substitutions/Insertions/Deletions include any one or more, or any two or more (e.g., any two), of the EGFR Protein Amino Acid Substitutions/Insertions/Deletions delineated in Table 1a, 1b and/or Table 2a, 2b; e.g., any one or more, or any two or more (e.g., any two), of the following and independently selected EGFR Protein Amino Acid Substitutions/Insertions/Deletions: V769L; V769M; M766delinsMASVx2; A767_V769dupASV; A767delinsASVDx3; A767delinsASVG; S768_V769insX; V769_D770insX; V769_D770insASV; D770delinsDN; D770delinsDNPH; D770_N771insSV; N771delinsNPH; N771_H773dup; L858R/C797S (or C797G); or Del_19 and C797S (or C797G), or any combination thereof.

As used herein, a “first inhibitor of EGFR” or “first EGFR inhibitor” is an EGFR inhibitor as defined herein, but which does not include the compound of Formula (I), or a pharmaceutically acceptable salt thereof, as defined herein. As used herein, a “second inhibitor of EGFR” or a “second EGFR inhibitor” is an EGFR inhibitor as defined herein, but which does not include the compound of Formula (I), or a pharmaceutically acceptable salt thereof, as defined herein. When both a first and a second inhibitor of EGFR are present in a method provided herein, the first and second inhibitors of EGFR are different. In some embodiments, the first and/or second inhibitor of EGFR bind in a different location than the compound of Formula (I). For example, in some embodiments, a first and/or second inhibitor of EGFR can inhibit dimerization of EGFR, while the compound of Formula (I) can inhibit the active site. In some embodiments, a first and/or second EGFR inhibitor can be an allosteric inhibitor of EGFR, while the compound of Formula (I) can inhibit the EGFR active site.

Exemplary first and second inhibitors of EGFR are described herein. In some embodiments, a first or second inhibitor of EGFR can be selected from the group consisting of osimertinib, gefitinib, erlotinib, afatinib, lapatinib, neratinib, AZD-9291, CL-387785, CO-1686, or WZ4002.

In some embodiments, the compound of Formula (I), or pharmaceutically acceptable salts and solvates thereof, are useful for treating a cancer that has been identified as having one or more EGFR inhibitor resistance mutations (that result in an increased resistance to a first or second inhibitor of EGFR, e.g., a substitution described in Table 2a and Table 2b including substitutions at amino acid position 747, 761, 790, 797, or 854 (e.g., L718Q, L747S, D761Y, T790M, C797S, T854A)). In some embodiments, the one or more EGFR inhibitor resistance mutations occurs in a nucleic acid sequence encoding a mutant EGFR protein (e.g., a mutant EGFR protein having any of the mutations described in Table 2a and Table 2b) resulting in a mutant EGFR protein that exhibits EGFR inhibitor resistance.

The epidermal growth factor receptor (EGFR) belongs to the ErbB family of receptor tyrosine kinases (RTKs) and provides critical functions in epithelial cell physiology (Schlessinger J (2014) Cold Spring Harb Perspect Biol 6, a008912). It is frequently mutated and/or overexpressed in different types of human cancers and is the target of multiple cancer therapies currently adopted in the clinical practice (Yarden Y and Pines G (2012) Nat Rev Cancer 12, 553-563).

Accordingly, provided herein are methods for treating a subject diagnosed with (or identified as having) a cancer that include administering to the subject a therapeutically effective amount of the compound of Formula (I), or a pharmaceutically acceptable salt thereof.

Also provided herein are methods for treating a subject identified or diagnosed as having an EGFR-associated cancer that include administering to the subject a therapeutically effective amount of the compound of Formula (I), or a pharmaceutically acceptable salt thereof, or a pharmaceutical composition thereof. In some embodiments, the subject that has been identified or diagnosed as having an EGFR-associated cancer through the use of a regulatory agency-approved, e.g., FDA-approved test or assay for identifying dysregulation of an EGFR gene, an EGFR kinase, or expression or activity or level of any of the same, in a subject or a biopsy sample from the subject or by performing any of the non-limiting examples of assays described herein. In some embodiments, the test or assay is provided as a kit. In some embodiments, the cancer is an EGFR-associated cancer. For example, the EGFR-associated cancer can be a cancer that includes one or more EGFR inhibitor resistance mutations.

The term “regulatory agency” refers to a country's agency for the approval of the medical use of pharmaceutical agents with the country. For example, a non-limiting example of a regulatory agency is the U.S. Food and Drug Administration (FDA).

Also provided are methods for treating cancer in a subject in need thereof, the method comprising: (a) detecting an EGFR-associated cancer in the subject; and (b) administering to the subject a therapeutically effective amount of the compound of Formula (I), or a pharmaceutically acceptable salt thereof, or a pharmaceutical composition thereof. Some embodiments of these methods further include administering to the subject another anticancer agent (e.g., a second EGFR inhibitor, or a pharmaceutically acceptable salt thereof, or an immunotherapy). In some embodiments, the subject was previously treated with a first EGFR inhibitor or previously treated with another anticancer treatment, e.g., at least partial resection of the tumor or radiation therapy. In some embodiments, the subject is determined to have an EGFR-associated cancer through the use of a regulatory agency-approved, e.g., FDA-approved test or assay for identifying dysregulation of an EGFR gene, an EGFR kinase, or expression or activity or level of any of the same, in a subject or a biopsy sample from the subject or by performing any of the non-limiting examples of assays described herein. In some embodiments, the test or assay is provided as a kit. In some embodiments, the cancer is an EGFR-associated cancer. For example, the EGFR-associated cancer can be a cancer that includes one or more EGFR inhibitor resistance mutations.

Also provided are methods of treating a subject that include performing an assay on a sample obtained from the subject to determine whether the subject has a dysregulation of an EGFR gene, an EGFR kinase, or expression or activity or level of any of the same, and administering (e.g., specifically or selectively administering) a therapeutically effective amount of the compound of Formula (I), or a pharmaceutically acceptable salt thereof, or a pharmaceutical composition thereof to the subject determined to have a dysregulation of an EGFR gene, an EGFR kinase, or expression or activity or level of any of the same. Some embodiments of these methods further include administering to the subject another anticancer agent (e.g., a second EGFR inhibitor, or a pharmaceutically acceptable salt thereof, or immunotherapy). In some embodiments of these methods, the subject was previously treated with a first EGFR inhibitor or previously treated with another anticancer treatment, e.g., at least partial resection of a tumor or radiation therapy. In some embodiments, the subject is a subject suspected of having an EGFR-associated cancer, a subject presenting with one or more symptoms of an EGFR-associated cancer, or a subject having an elevated risk of developing an EGFR-associated cancer. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art. In some embodiments, the dysregulation of an EGFR gene, an EGFR kinase, or expression or activity or level of any of the same includes one or more EGFR inhibitor resistance mutations.

Also provided is the compound of Formula (I), or a pharmaceutically acceptable salt thereof, or a pharmaceutical composition thereof, for use in treating an EGFR-associated cancer in a subject identified or diagnosed as having an EGFR-associated cancer through a step of performing an assay (e.g., an in vitro assay) on a sample obtained from the subject to determine whether the subject has a dysregulation of an EGFR gene, an EGFR kinase, or expression or activity or level of any of the same, where the presence of a dysregulation of an EGFR gene, an EGFR kinase, or expression or activity or level of any of the same, identifies that the subject has an EGFR-associated cancer. Also provided is the use of the compound of Formula (I), or a pharmaceutically acceptable salt thereof, for the manufacture of a medicament for treating an EGFR-associated cancer in a subject identified or diagnosed as having an EGFR-associated cancer through a step of performing an assay on a sample obtained from the subject to determine whether the subject has a dysregulation of an EGFR gene, an EGFR kinase, or expression or activity or level of any of the same where the presence of dysregulation of an EGFR gene, an EGFR kinase, or expression or activity or level of any of the same, identifies that the subject has an EGFR-associated cancer. Some embodiments of any of the methods or uses described herein further include recording in the subject's clinical record (e.g., a computer readable medium) that the subject is determined to have a dysregulation of an EGFR gene, an EGFR kinase, or expression or activity or level of any of the same, through the performance of the assay, should be administered the compound of Formula (I), or a pharmaceutically acceptable salt thereof, or a pharmaceutical composition thereof. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. In some embodiments, the dysregulation of an EGFR gene, an EGFR kinase, or expression or activity or level of any of the same includes one or more EGFR inhibitor resistance mutations.

Also provided is the compound of Formula (I), or a pharmaceutically acceptable salt thereof, for use in the treatment of a cancer in a subject in need thereof or a subject identified or diagnosed as having an EGFR-associated cancer. Also provided is the use of the compound of Formula (I), or a pharmaceutically acceptable salt thereof, for the manufacture of a medicament for treating a cancer in a subject identified or diagnosed as having an EGFR-associated cancer. In some embodiments, the cancer is an EGFR-associated cancer, for example, an EGFR-associated cancer having one or more EGFR inhibitor resistance mutations. In some embodiments, a subject is identified or diagnosed as having an EGFR-associated cancer through the use of a regulatory agency-approved, e.g., FDA-approved, kit for identifying dysregulation of an EGFR gene, an EGFR kinase, or expression or activity or level of any of the same, in a subject or a biopsy sample from the subject. As provided herein, an EGFR-associated cancer includes those described herein and known in the art.

In some embodiments of any of the methods or uses described herein, the subject has been identified or diagnosed as having a cancer with a dysregulation of an EGFR gene, an EGFR kinase, or expression or activity or level of any of the same. In some embodiments of any of the methods or uses described herein, the subject has a tumor that is positive for a dysregulation of an EGFR gene, an EGFR kinase, or expression or activity or level of any of the same. In some embodiments of any of the methods or uses described herein, the subject can be a subject with a tumor(s) that is positive for a dysregulation of an EGFR gene, an EGFR kinase, or expression or activity or level of any of the same. In some embodiments of any of the methods or uses described herein, the subject can be a subject whose tumors have a dysregulation of an EGFR gene, an EGFR kinase, or expression or activity or level of any of the same. In some embodiments of any of the methods or uses described herein, the subject is suspected of having an EGFR-associated cancer (e.g., a cancer having one or more EGFR inhibitor resistance mutations). In some embodiments, provided herein are methods for treating an EGFR-associated cancer in a subject in need of such treatment, the method comprising a) detecting a dysregulation of an EGFR gene, an EGFR kinase, or the expression or activity or level of any of the same in a sample from the subject; and b) administering a therapeutically effective amount of the compound of Formula (I), or a pharmaceutically acceptable salt thereof. In some embodiments, the dysregulation of an EGFR gene, an EGFR kinase, or the expression or activity or level of any of the same includes one or more EGFR kinase protein point mutations/insertions/deletions. Non-limiting examples of EGFR kinase protein point mutations/insertions/deletions are described in Table 1a and Table 1b. In some embodiments, the EGFR kinase protein point mutations/insertions/deletions are selected from the group consisting of G719S, G719C, G719A, L747S, D761Y, T790M, T854A, L858R, L861Q, a deletion in exon 19 (e.g., L747_A750del), and an insertion in exon 20. In some embodiments, the EGFR kinase protein point mutations/insertions/deletions are selected from the group consisting of L858R, deletions in exon 19 (e.g., L747_A750del), L747S, D761Y, T790M, and T854A. In some embodiments, the dysregulation of an EGFR gene, an EGFR kinase, or the expression or activity or level of any of the same includes one or more EGFR inhibitor resistance mutations. Non-limiting examples of EGFR inhibitor resistance mutations are described in Table 2a and Table 2b. In some embodiments, the EGFR inhibitor resistance mutation is a substitution at amino acid position 718, 747, 761, 790, 797, or 854 (e.g., L718Q, L747S, D761Y, T790M, C797S, and T854A). In some embodiments, the dysregulation of an EGFR gene, an EGFR kinase, or the expression or activity or level of any of the same includes one or more point mutations/insertions/deletions in exon 20. Non-limiting examples of EGFR exon 20 mutations are described in Tables 1a, 1b, 2a and 2b. In some embodiments, the EGFR exon 20 mutation is an exon 20 insertion such as V769_D770insX, D770_N771insX, N771_P772insX, P772_H773insX, and H773_V774insX. For example, the EGFR kinase protein insertion is an exon 20 insertion selected from the group consisting of: A767_V769dupASV, V769_D770insASV, D770_N771insNPG, D770_N771insNPY, D770_N771insSVD, D770_N771insGL, N771_H773dupNPH, N771_P772insN, N771_P772insH, N771_P772insV, P772_H773insDNP, P772_H773insPNP, H773_V774insNPH, H773_V774insH, H773_V774insPH, H773_V774insAH, and P772_H773insPNP. In some embodiments, the cancer with a dysregulation of an EGFR gene, an EGFR kinase, or expression or activity or level of any of the same is determined using a regulatory agency-approved, e.g., FDA-approved, assay or kit. In some embodiments, the tumor that is positive for a dysregulation of an EGFR gene, an EGFR kinase, or expression or activity or level of any of the same is a tumor positive for one or more EGFR inhibitor resistance mutations. In some embodiments, the tumor with a dysregulation of an EGFR gene, an EGFR kinase, or expression or activity or level of any of the same is determined using a regulatory agency-approved, e.g., FDA-approved, assay or kit.

In some embodiments of any of the methods or uses described herein, the subject has a clinical record indicating that the subject has a tumor that has a dysregulation of an EGFR gene, an EGFR kinase, or expression or activity or level of any of the same (e.g., a tumor having one or more EGFR inhibitor resistance mutations). Also provided are methods of treating a subject that include administering a therapeutically effective amount of the compound of Formula (I), or a pharmaceutically acceptable salt thereof, to a subject having a clinical record that indicates that the subject has a dysregulation of an EGFR gene, an EGFR kinase, or expression or activity or level of any of the same.

In some embodiments, the methods provided herein include performing an assay on a sample obtained from the subject to determine whether the subject has a dysregulation of an EGFR gene, an EGFR protein, or expression or level of any of the same. In some such embodiments, the method also includes administering to a subject determined to have a dysregulation of an EGFR gene, an EGFR protein, or expression or activity, or level of any of the same a therapeutically effective amount of the compound of Formula (I), or a pharmaceutically acceptable salt thereof. In some embodiments, the method includes determining that a subject has a dysregulation of an EGFR gene, an EGFR protein, or expression or level of any of the same via an assay performed on a sample obtained from the subject. In such embodiments, the method also includes administering to a subject a therapeutically effective amount of the compound of Formula (I), or a pharmaceutically acceptable salt thereof. In some embodiments, the dysregulation in an EGFR gene, an EGFR kinase protein, or expression or activity or level of any of the same is one or more point mutation in the EGFR gene (e.g., any of the one or more of the EGFR point mutations described herein). The one or more point mutations in an EGFR gene can result, e.g., in the translation of an EGFR protein having one or more of the following amino acid substitutions, deletions, and insertions: G719S, G719C, G719A, L747S, D761Y, T790M, T854A, L858R, L861Q, a deletion in exon 19 (e.g., L747_A750del), and an insertion in exon 20 (e.g., V769_D770insX, D770_N771insX, N771_P772insX, P772_H773insX, and H773_V774insX). The one or more mutations in an EGI-R gene can result, e.g., in the translation of an EGFR protein having one or more of the following amino acid substitutions or deletions: L858R, deletions in exon 19 (e.g., L747_A750del), L747S, D761Y, T790M, and T854A. In some embodiments, the dysregulation in an EGFR gene, an EGFR kinase protein, or expression or activity or level of any of the same is one or more EGFR inhibitor resistance mutations (e.g., any combination of the one or more EGFR inhibitor resistance mutations described herein). In some embodiments, the dysregulation in an EGFR gene, an EGFR kinase protein, or expression or activity or level of any of the same is one or more EGFR exon 20 insertions (e.g., any of the exon 20 insertions described herein). In some embodiments, the EGFR kinase protein insertion is an exon 20 insertion selected from the group consisting of: V769_D770insX, D770_N771insX, N771_P772insX, P772_H773insX, and H773_V774insX. In some embodiments, the EGFR kinase protein insertion is an exon 20 insertion selected from the group consisting of: V769_D770insX, D770_N771insX, N771_P772insX, P772_H773insX, and H773_V774insX. In some embodiments, the EGFR kinase protein insertion is an exon 20 insertion selected from the group consisting of: A767_V769dupASV, V769_D770insASV, D770 N771insNPG, D770 N771insNPY, D770 N771insSVD, D770 N771insGL, N771_H773dupNPH, N771_P772insN, N771_P772insH, N771_P772insV, P772_H773insDNP, P772_H773insPNP, H773_V774insNPH, H773_V774insH, H773_V774insPH, H773_V774insAH, and P772_H773insPNP. Some embodiments of these methods further include administering to the subject another anticancer agent (e.g., a second EGFR inhibitor, or a pharmaceutically acceptable salt thereof, or immunotherapy).

In some embodiments of any of the methods or uses described herein, an assay used to determine whether the subject has a dysregulation of an EGFR gene, or an EGFR kinase, or expression or activity or level of any of the same, using a sample from a subject can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labelled nucleic acid probe or at least one labelled antibody or antigen-binding fragment thereof. Assays can utilize other detection methods known in the art for detecting dysregulation of an EGFR gene, an EGFR kinase, or expression or activity or levels of any of the same (see, e.g., the references cited herein). In some embodiments, the dysregulation of the EGFR gene, the EGFR kinase, or expression or activity or level of any of the same includes one or more EGFR inhibitor resistance mutations. In some embodiments, the sample is a biological sample or a biopsy sample (e.g., a paraffin-embedded biopsy sample) from the subject. In some embodiments, the subject is a subject suspected of having an EGFR-associated cancer, a subject having one or more symptoms of an EGFR-associated cancer, and/or a subject that has an increased risk of developing an EGFR-associated cancer).

In some embodiments, dysregulation of an EGFR gene, an EGFR kinase, or the expression or activity or level of any of the same can be identified using a liquid biopsy (variously referred to as a fluid biopsy or fluid phase biopsy). See, e.g., Karachialiou et al., “Real-time liquid biopsies become a reality in cancer treatment”, Ann. Transl. Med., 3(3):36, 2016. Liquid biopsy methods can be used to detect total tumor burden and/or the dysregulation of an EGFR gene, an EGFR kinase, or the expression or activity or level of any of the same. Liquid biopsies can be performed on biological samples obtained relatively easily from a subject (e.g., via a simple blood draw) and are generally less invasive than traditional methods used to detect tumor burden and/or dysregulation of an EGFR gene, an EGFR kinase, or the expression or activity or level of any of the same. In some embodiments, liquid biopsies can be used to detect the presence of dysregulation of an EGFR gene, an EGFR kinase, or the expression or activity or level of any of the same at an earlier stage than traditional methods. In some embodiments, the biological sample to be used in a liquid biopsy can include, blood, plasma, urine, cerebrospinal fluid, saliva, sputum, broncho-alveolar lavage, bile, lymphatic fluid, cyst fluid, stool, ascites, and combinations thereof. In some embodiments, a liquid biopsy can be used to detect circulating tumor cells (CTCs). In some embodiments, a liquid biopsy can be used to detect cell-free DNA. In some embodiments, cell-free DNA detected using a liquid biopsy is circulating tumor DNA (ctDNA) that is derived from tumor cells. Analysis of ctDNA (e.g., using sensitive detection techniques such as, without limitation, next-generation sequencing (NGS), traditional PCR, digital PCR, or microarray analysis) can be used to identify dysregulation of an EGFR gene, an EGFR kinase, or the expression or activity or level of any of the same.

The term “HER2-associated disease or disorder” as used herein refers to diseases or disorders associated with or having a dysregulation of a HER2 gene, a HER2 kinase, or the expression or activity or level of any (e.g., one or more) of the same (e.g., any of the types of dysregulation of a HER2 gene, a HER2 kinase, a HER2 kinase domain, or the expression or activity or level of any of the same described herein). Non-limiting examples of a HER2-associated disease or disorder include, for example, cancer.

The term “HER2-associated cancer” as used herein refers to cancers associated with or having a dysregulation of a HER2 gene, a HER2 kinase (also called herein a HER2 protein), or expression or activity, or level of any of the same. Non-limiting examples of a HER2-associated cancer are described herein.

In some embodiments, the EGFR-associated cancer is also a HER2-associated cancer. For example, an EGFR-associated cancer can also have a dysregulation of a HER2 gene, a HER2 kinase, or the expression or activity or level of any of the same.

The phrase “dysregulation of a HER2 gene, a HER2 kinase, or the expression or activity or level of any of the same” refers to a genetic mutation (e.g., a mutation in a HER2 gene that results in the expression of a HER2 protein that includes a deletion of at least one amino acid as compared to a wild type HER2 protein, a mutation in a HER2 gene that results in the expression of a HER2 protein with one or more point mutations as compared to a wild type HER2 protein, a mutation in a HER2 gene that results in the expression of a HER2 protein with at least one inserted amino acid as compared to a wild type HER2 protein, a gene duplication that results in an increased level of HER2 protein in a cell, or a mutation in a regulatory sequence (e.g., a promoter and/or enhancer) that results in an increased level of HER2 protein in a cell), an alternative spliced version of a HER2 mRNA that results in a HER2 protein having a deletion of at least one amino acid in the HER2 protein as compared to the wild-type HER2 protein), or increased expression (e.g., increased levels) of a wild type HER2 kinase in a mammalian cell due to aberrant cell signaling and/or dysregulated autocrine/paracrine signaling (e.g., as compared to a control non-cancerous cell). As another example, a dysregulation of a HER2 gene, a HER2 protein, or expression or activity, or level of any of the same, can be a mutation in a HER2 gene that encodes a HER2 protein that is constitutively active or has increased activity as compared to a protein encoded by a HER2 gene that does not include the mutation. Non-limiting examples of HER2 kinase protein fusions and point mutations/insertions/deletions are described in Tables 3-5. Such mutation and overexpression is associated with the development of a variety of cancers (Moasser. Oncogene. 2007 Oct. 4; 26(45): 6469-6487).

The compounds of Formula (I), or pharmaceutically acceptable salts or solvates thereof, are useful for treating diseases and disorders such as HER2-associated diseases and disorders, e.g., proliferative disorders such as cancers, including hematological cancers and solid tumors (e.g., advanced solid tumors).

In some embodiments, dysregulation of a HER2 gene, a HER2 kinase, or the expression or activity or level of any of the same can be caused by an activating mutation in a HER2 gene. The exemplary HER2 kinase fusions or point mutations, insertions, and deletions shown in Tables 3-5 can be caused by an activating mutation The term “activating mutation” in reference to HER2 describes a mutation in a HER2 gene that results in the expression of a HER2 kinase that has an increased kinase activity, e.g., as compared to a wild type HER2 kinase, e.g., when assayed under identical conditions. For example, an activating mutation can be a mutation in a HER2 gene (that results in the expression of a HER2 kinase that has one or more (e.g., two, three, four, five, six, seven, eight, nine, or ten) amino acid substitutions (e.g., any combination of any of the amino acid substitutions described herein) that has increased kinase activity, e.g., as compared to a wild type HER2 kinase, e.g., when assayed under identical conditions. In another example, an activating mutation can be a mutation in a HER2 gene that results in the expression of a HER2 kinase that has one or more (e.g., two, three, four, five, six, seven, eight, nine, or ten) amino acids deleted, e.g., as compared to a wild type HER2 kinase, e.g., when assayed under identical conditions. In another example, an activating mutation can be a mutation in a HER2 gene that results in the expression of a HER2 kinase that has at least one (e.g., at least 2, at least 3, at least 4, at least 5, at least 6, at least 7, at least 8, at least 9, at least 10, at least 12, at least 14, at least 16, at least 18, or at least 20) amino acid inserted as compared to a wild type HER2 kinase, e.g., the exemplary wild type HER2 kinase described herein, e.g., when assayed under identical conditions. Additional examples of activating mutations are known in the art.

The term “wild type HER2” or “wild-type HER2 kinase” describes a HER2 nucleic acid (e.g., a HER2 gene or a HER2 mRNA) or protein (e.g., a HER2 protein) that is found in a subject that does not have a HER2-associated disease, e.g., a HER2-associated cancer (and optionally also does not have an increased risk of developing a HER2-associated disease and/or is not suspected of having a HER2-associated disease), or is found in a cell or tissue from a subject that does not have a HER2-associated disease, e.g., a HER2-associated cancer (and optionally also does not have an increased risk of developing a HER2-associated disease and/or is not suspected of having a HER2-associated disease).

Provided herein is a method of treating a HER2-associated cancer (in a subject in need of such treatment, the method comprising administering to the subject a therapeutically effective amount of the compound of Formula (I), or a pharmaceutically acceptable salt thereof or a pharmaceutical composition thereof. For example, provided herein are methods for treating a HER2-associated cancer in a subject in need of such treatment, the method comprising a) detecting a dysregulation of a HER2 gene, a HER2 kinase, or the expression or activity or level of any of the same in a sample from the subject; and b) administering a therapeutically effective amount of the compound of Formula (I), or a pharmaceutically acceptable salt thereof. In some embodiments, the dysregulation of a HER2 gene, a HER2 kinase, or the expression or activity or level of any of the same includes one or more HER2 kinase protein point mutations/insertions. Non-limiting examples of HER2 kinase protein fusions and point mutations/insertions/deletions are described in Tables 3-5. In some embodiments, the HER2 kinase protein point mutations/insertions/deletions are selected from the group consisting of S310F, S310Y, R678Q, R678W, R678P, I767M, V773M, V777L, V842I, Y772_A775dup, A775_G776insYVMA, G776delinsVC, G776delinsVV, V777_G778insGSP, and P780_Y781insGSP. In some embodiments, the HER2 kinase protein point mutations/insertions/deletions are exon 20 point mutations/insertions/deletions selected from the group consisting of V773M, G776C, G776V, G776S, V777L, V777M, S779T, P780L, S783P, M774AYVM, M774del insWLV, A775_G776insYVMA, A775_G776insAVMA, A775_G776insSVMA, A775_G776insVAG, A775insV G776C, A775_G776insI, G776del insVC2, G776del insVV, G776del insLC, G776C V777insC, G776C V777insV, V777_G778insCG, G778_S779insCPG, and P780_Y781insGSP. In some embodiments, the HER2 kinase protein point mutations/insertions/deletions are exon 20 point mutations/insertions/deletions selected from the group consisting of Y772_A775dup, A775_G776insYVMA, G776delinsVC, G776delinsVV, V777_G778insGSP, and P780_Y781insGSP.

In some embodiments of any of the methods or uses described herein, the cancer (e.g., HER2-associated cancer) is selected from a hematological cancer (e.g., Hodgkin lymphoma, non-Hodgkin lymphoma, and leukemia such as acute-myelogenous leukemia (AML), chronic-myelogenous leukemia (CML), acute-promyelocytic leukemia, and acute lymphocytic leukemia (ALL)), alveolar rhabdomyosarcoma, central or peripheral nervous system tissue cancer, an endocrine or neuroendocrine cancer including multiple neuroendocrine type I and type II tumors, Li-Fraumeni tumors, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, tracheal cancer, oral cancer, oropharyngeal cancer, nasopharyngeal cancer, respiratory cancer, urogenital cancer, cancer of the vulva, colon cancer, esophageal cancer, cervical cancer, gastrointestinal carcinoid tumor, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer including pancreatic islet cell cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, parathyroid cancer, pituitary tumors, adrenal gland tumors, ureter cancer, biliary cancer, and urinary bladder cancer. In some embodiments, the cancer is selected from the group consisting of: head and neck, ovarian, cervical, bladder and oesophageal cancers, pancreatic, gastrointestinal cancer, gastric, breast, endometrial and colorectal cancers, hepatocellular carcinoma, glioblastoma, bladder, lung cancer, e.g., non-small cell lung cancer (NSCLC), bronchioloalveolar carcinoma. In some embodiments, the cancer is pancreatic cancer, head and neck cancer, melanoma, colon cancer, renal cancer, leukemia, lung cancer, or breast cancer. In some cases, the cancer is melanoma, colon cancer, renal cancer, leukemia, or breast cancer.

In some such embodiments, the compounds provided herein are useful for treating a primary brain tumor or metastatic brain tumor. For example, the compounds can be used in the treatment of one or more of gliomas such as glioblastoma (also known as glioblastoma multiforme), astrocytomas, oligodendrogliomas, ependymomas, and mixed gliomas, meningiomas, medulloblastomas, gangliogliomas, schwannomas (neurilemmomas), and craniopharyngiomas (see, for example, Liu et al. J Exp Clin Cancer Res. 2019 May 23; 38(1):219); and Ding et al. Cancer Res. 2003 Mar. 1; 63(5):1106-13). In some embodiments, the brain tumor is a primary brain tumor. In some embodiments, the brain tumor is a metastatic brain tumor, e.g., a metastatic brain tumor from lung cancer, melanoma, breast cancer, ovarian cancer, colorectal cancer, kidney cancer, bladder cancer, or undifferentiated carcinoma. In some embodiments, the brain tumor is a metastatic brain tumor from lung cancer (e.g., non-small cell lung cancer). In some embodiments, the compounds provided herein exhibit brain and/or central nervous system (CNS) penetrance. In some embodiments, the patient has previously been treated with another anticancer agent, e.g., another EGFR and/or HER2 inhibitor (e.g., a compound that is not a compound of Formula I) or a multi-kinase inhibitor.

In some embodiments, the cancer is a cancer of B cell origin. In some embodiments, the cancer is a lineage dependent cancer. In some embodiments, the cancer is a lineage dependent cancer where HER2 or the dysregulation of an HER2 gene, an HER2 kinase, or expression or activity or level of any of the same, plays a role in the initiation and/or development of the cancer.

Also provided herein is a method for treating a subject diagnosed with or identified as having a HER2-associated cancer, e.g., any of the exemplary HER2-associated cancers disclosed herein, comprising administering to the subject a therapeutically effective amount of the compound of Formula (I), or a pharmaceutically acceptable salt thereof, or a pharmaceutical composition thereof. as defined herein.

In some embodiments, the dysregulation of a HER2 gene, a HER2 kinase, or expression or activity or level of any of the same, includes one or more deletions (e.g., deletion of an amino acid at position 12), insertions, or point mutation(s) in a HER2 kinase. In some embodiments, the dysregulation of a HER2 gene, a HER2 kinase, or expression or activity or level of any of the same, includes a deletion of one or more residues from the HER2 kinase, resulting in increased signaling activity of HER2.

In some embodiments, the dysregulation of a HER2 gene, a HER2 kinase, or expression or activity or level of any of the same, includes at least one point mutation in a HER2 gene that results in the production of a HER2 kinase that has one or more amino acid substitutions, insertions, or deletions as compared to the wild-type HER2 kinase (see, for example, the point mutations listed in Table 3). In some embodiments, dysregulation of a HER2 gene, a HER2 kinase, or expression or activity or level of any of the same, includes at least one point mutation in a HER2 gene that results in the production of a HER2 kinase that has one or more of the amino acid substitutions, insertions, or deletions in Table 3.

In some embodiments, the dysregulation of an HER2 gene, an HER2 kinase, or expression or activity or level of any of the same, includes an insertion of one or more residues in exon 20 of the HER2 gene (e.g., any of the exon 20 insertions described in Table 1a and Table 1b). Exon 20 of HER2 has two major regions, the c-helix (residues 770-774) and the loop following the c-helix (residues 775-783). In some embodiments, the dysregulation of an HER2 gene, an HER2 kinase, or expression or activity or level of any of the same, includes an insertion of one or more residues in exon 20 selected from the group consisting of: Y772_A775dup, A775_G776insYVMA, G776delinsVC, G776delinsVV, V777_G778insGSP, and P780_Y781insGSP.

TABLE 3
HER2 Protein Amino Acid Substitutions/Insertions/Deletions^{A}

| Amino Acid | Non-Limiting | Non-limiting Exemplary HER2- |
|---|---|---|
| Position(s) | Exemplary Mutations | associated Cancer(s) |
| 122 | P122L¹¹ | Metastatic Colorectal Cancer |
| 263 | I263T⁷ | Colorectal Cancer⁷ |
| 265 | E265K¹¹ | Metastatic Colorectal Cancer |
| 270 | A270S⁶ | Breast Cancer |
| 292 | G292R¹¹ | Metastatic Colorectal Cancer |
| 309 | G309A³, G309E¹⁵ | Breast Cancer |
| 310 | S310F⁷, S310Y⁸ | Colorectal Cancer⁷ |
| 311 | C311R⁸ | |
| 313 | L313V¹¹ | Metastatic Colorectal Cancer |
| 429 | S429R¹⁵, S429H¹⁵ | |
| 466 | A466T⁷ | Colorectal Cancer⁷ |
| 648 | A648V¹⁰ | Urinary Cancer |
| 650 | P650L¹⁰, P650S¹⁰ | Melanoma, Uterine Cancer |
| 651 | L651V¹⁰ | Breast Cancer, Cervical Cancer |
| 652 | T652M¹⁰, T652R¹⁰ | Lung Cancer, Colorectal Cancer, |
| | | Liver Cancer, Head And Neck |
| | | Cancer, Endometrial Cancer, |
| | | Ovarian Cancer |
| 653 | S653C¹⁰, S653P¹⁰ | Breast Cancer¹⁰, Urinary Cancer, |
| | | Breast Cancer, Colorectal Cancer, |
| | | Liver Cancer, Metastatic |
| | | Colorectal Cancer¹¹ |
| 654 | I654T¹⁰, I654M¹⁰, I654L¹⁰ | Lower Gastrointestinal Cancer, |
| | | Neuroendocrine Cancer, Breast |
| | | Cancer, Esophageal Cancer, Soft |
| | | Tissue Cancer |
| 655 | I655M¹⁰, I655V¹⁴ | Lung Cancer, Colorectal Cancer, |
| | | Ovarian Cancer, Urinary Cancer |
| 656 | S656C¹⁰ | Esophageal Cancer |
| 657 | A657V¹⁰ | Prostate Cancer, Colorectal Cancer |
| 659 | V659E¹⁰, V659D¹⁰, V659L¹⁰, | Lung cancer¹⁰, Biliary Cancer, |
| | V659F¹⁰, | Colorectal Cancer, Breast Cancer, |
| | | Metastatic Colorectal Cancer¹¹ |
| 659-661 | V659_I661 > VVEGI¹⁰ | Lung Cancer |
| 659-660 | V659_G660 > ER¹⁰ | Lung Cancer |
| 660 | G660D¹⁰ | Biliary Cancer¹⁰, Lung Cancer, |
| | | Urinary Cancer, Colorectal Cancer, |
| | | Glioma, Lower Gastrointestinal |
| | | Cancer, Gastric Cancer, Liver |
| | | Cancer, Metastatic Colorectal |
| | | Cancer¹¹ |
| 661 | I661V¹⁰ | Colorectal Cancer |
| 662 | L662V¹⁰ | Cervical Cancer, Lung Cancer, |
| | | Breast Cancer |
| 663 | L663P¹⁰ | Soft Tissue Cancer |
| 664 | V664F¹⁰, V664I¹⁰ | Lung Cancer, Breast Cancer, |
| | | Gastric Cancer |
| 665 | V665M¹⁰ | Prostate Cancer, Colorectal Cancer |
| 665-666 | V665_V666del¹⁰ | Breast Cancer |
| 666 | V666I¹⁰ | Colorectal Cancer |
| 667 | L667*¹⁰, L667S¹⁰ | Breast Cancer, Soft Tissue Cancer |
| 668 | G668E¹⁰, G668R¹⁰ | Glioma, Lung Cancer |
| 669 | V669A¹⁰, V669L¹⁰ | Biliary Cancer, Breast Cancer, |
| | | Glioma |
| 672 | G672R¹⁰ | SSC other, Lung Cancer |
| 673 | I673F¹⁰, I673M¹⁰, I673V¹⁰ | Colorectal Cancer, Lung Cancer |
| 674 | L674V¹⁰, L674I¹⁰ | Colorectal Cancer, Lung Cancer |
| 675 | I675M¹⁰, I675T¹⁰, I675L¹⁰ | Lung Cancer, Urinary Cancer, |
| | | Thyroid Cancer, |
| 676 | K676M¹⁰ | Lung Cancer |
| 677 | R677*¹⁰, R677L¹⁰, R677Q¹⁰ | Lung Cancer, Endometrial Cancer, |
| | | Pancreatic Cancer, Neuroendocrine |
| | | Cancer, Colorectal Cancer, |
| | | Glioma, Myeloma |
| 678 | R678Q⁷, R678W¹⁰, R678P¹⁰ | Colorectal Cancer⁷, Gastric |
| | | Cancer¹⁰, Biliary Cancer¹⁰, Urinary |
| | | Cancer¹⁰, Ovarian Cancer¹⁰, |
| | | Endometrial Cancer¹⁰, Carcinoma |
| | | of Unknown Primary¹⁰, Lung |
| | | Cancer¹⁰, Appendicidal Cancer¹⁰, |
| | | Pancreatic Cancer¹⁰, Breast Cancer¹⁰, |
| | | Neuroendocrine Cancer¹⁰, |
| | | Cervical Cancer¹⁰, Lower |
| | | Gastrointestinal Cancer¹⁰, Prostate |
| | | Cancer¹⁰, Liver Cancer¹⁰, Central |
| | | Nervous System Cancer (Non- |
| | | Glioma)¹⁰, Melanoma¹⁰, Salivary |
| | | Gland Cancer¹⁰, Metastatic |
| | | Colorectal Cancer¹¹ |
| 679 | Q679E¹⁰, Q679H¹⁰ | Pancreatic Cancer, Colorectal |
| | | Cancer |
| 680 | Q680del¹⁰ | Cervical Cancer |
| 681 | K681N¹⁰ | Lung Cancer |
| 682 | I682T¹⁰, I682M¹⁰ | Endometrial Cancer, Colorectal |
| | | Cancer |
| 683 | R683W¹⁰, R683Q¹⁰ | Breast Cancer, Pancreatic Cancer, |
| | | Endometrial Cancer, Bone Cancer, |
| | | Colorectal Cancer, |
| 684 | K684N¹⁰ | Colorectal Cancer |
| 685 | Y685H¹⁰ | Colorectal Cancer, Carcinoma of |
| | | Unknown Primary |
| 686 | T686M¹⁰, T686A¹⁰, T686R¹⁰ | Colorectal Cancer, Urinary Cancer, |
| | | Ovarian Cancer, Endometrial |
| | | Cancer |
| 688 | R688L¹⁰, R688W¹⁰, R688Q¹⁰ | Skin Cancer, Colorectal Cancer, |
| | | Melanoma |
| 689 | R689K¹⁰, R689I¹⁰ | Lung Cancer, Endometrial Cancer |
| 691 | L691R¹⁰ | Endometrial Cancer |
| 693 | E693K¹⁰, E693G¹⁰ | Ovarian Cancer, Melanoma, |
| | | Colorectal Cancer, Breast Cancer, |
| | | Carcinoma of Unknown Primary |
| 694 | T694M¹⁰, T694S¹⁰ | Carcinoma of Unknown Primary, |
| | | Colorectal Cancer, Pancreatic |
| | | Cancer |
| 695 | E695K¹⁰ | Melanoma, Urinary Cancer |
| 697 | V697L¹⁰, V697M¹⁰, | Breast Cancer, Lung Cancer, |
| | V697del¹⁰ | Ovarian Cancer, Lower |
| | | Gastrointestinal Cancer, Colorectal |
| | | Cancer, Skin Cancer, Pancreatic |
| | | Cancer, Salivary Gland Cancer, |
| | | Carcinoma of Unknown Primary, |
| | | Cervical Cancer, Endometrial |
| | | Cancer, Gastric Cancer, |
| 699 | P699del¹⁰, P699S | Endometrial Cancer |
| 701 | T701I¹⁰ | Ovarian Cancer |
| 702 | P702S¹⁰, P702L¹⁰ | Gastric Cancer, Lower |
| | | Gastrointestinal Cancer, |
| | | Carcinoma of Unknown Primary, |
| | | Endometrial Cancer, Breast |
| | | Cancer, Ovarian Cancer, Liver |
| | | Cancer |
| 704 | G704E¹⁰, G704R¹⁰ | Glioma, Colorectal Cancer |
| 705 | A705V¹⁰ | Colorectal Cancer, Soft Tissue |
| | | Cancer |
| 706 | M706V¹⁰ | Breast Cancer |
| 707 | P707L¹⁰ | Soft Tissue Cancer |
| 709 | Q709L¹⁰, Q709K¹⁰ | Glioma, Lung Cancer, Lower |
| | | Gastrointestinal Cancer |
| 710 | A710V¹⁰ | Lung Cancer |
| 711 | Q711H¹⁰ | Breast Cancer, Lung Cancer |
| 712 | M712L¹⁰ | Neuroendocrine Cancer, |
| | | Esophageal Cancer |
| 713 | R713Q¹⁰, R713W¹⁰, R713L¹⁰ | Prostate Cancer, Lung Cancer, |
| | | Endometrial Cancer |
| 726 | L726I¹⁵, L726F¹⁵ | |
| 733 | 733I¹¹ | Metastatic Colorectal Cancer¹¹ |
| 755 | L755S^{2,7}, L755W³, L755P⁸, | Lung Adenocarcinoma², Breast |
| | L755F¹⁴ | Cancer³, Bone Metastases From |
| | | Breast Cancer⁵, Colorectal |
| | | Cancer⁷, Metastatic Colorectal |
| | | Cancer¹¹ |
| 755-759 | del. 755-759³ | Breast Cancer³ |
| 760 | S760A¹⁵ | |
| 767 | I767M¹¹ | Metastatic Colorectal Cancer¹¹ |
| 769 | D769H², D769Y³, D769N¹¹ | Lung Adenocarcinoma², Breast |
| | | Cancer³, Metastatic Colorectal |
| | | Cancer¹¹ |
| 773 | V773M¹¹ | Metastatic Colorectal Cancer¹¹ |
| 776 | G776C², G776V⁸, G776S¹¹ | Lung Adenocarcinoma, Metastatic |
| | | Colorectal Cancer¹¹ |
| 777 | V777L^{2,7}, V777M² | Lung Adenocarcinoma², Breast |
| | | Cancer³, Bone Metastases From |
| | | Breast Cancer⁵, Colorectal |
| | | Cancer⁷, Metastatic Colorectal |
| | | Cancer¹¹ |
| 779 | S779T⁸ | Breast Cancer |
| 780 | P780ins³, P780L¹⁵ | Breast Cancer³ |
| 783 | S783P2 | Breast Cancer |
| 784 | R784G¹² | Metastatic Colorectal Cancer¹² |
| 785 | L785F¹⁵ | Breast Cancer |
| 798 | T798I⁴, T798M¹⁵ | Breast Cancer |
| 835 | Y835F¹⁵ | |
| 838 | R838Q¹⁵ | |
| 842 | V842I^{3,7} | Breast Cancer³, Colorectal |
| | | Cancer⁷, Metastatic Colorectal |
| | | Cancer¹¹ |
| 862 | T862A¹¹ | Metastatic Colorectal Cancer¹¹ |
| 866 | 866M⁷ | Colorectal Cancer⁷ |
| 868 | R868W⁷ | Colorectal Cancer⁷ |
| 869 | L869R⁴ | Breast Cancer |
| 869 + 798 | L869R + T798I⁴ | Breast Cancer |
| 878 | H878Y¹¹ | Metastatic Colorectal Cancer¹¹ |
| 887 | M887I¹⁵ | |
| 896 | R896C³, R896H¹¹ | Breast Cancer³, Metastatic |
| | | Colorectal Cancer¹¹ |
| 896 + 755 | R896C + L755W³ | Breast Cancer |
| 1136 | Q1136fs*5¹¹ | Metastatic Colorectal Cancer¹¹ |
| 1170 | P1170fs*88+¹¹ | Metastatic Colorectal Cancer¹¹ |
| 1189 | G1189fs*9¹¹ | Metastatic Colorectal Cancer¹¹ |
| 1201 | G120IV¹⁵ | |
| 1219 | N1219S⁷ | Colorectal Cancer⁷ |
| 1232 | A1232fs*25+¹¹ | Metastatic Colorectal Cancer¹¹ |
| Exon 16 | Deletion¹¹ | Metastatic Colorectal Cancer¹¹ |

Exon 20 deletions and insertions

| | |
|---|---|
| M774AYVM¹³ | Non-Small Cell Lung Cancer¹³ |
| M774delinsWLV⁹ | Non-Small Cell Lung Cancer⁹ |
| A775_G776insYVMA (c. | Lung Adenocarcinoma |
| 2324_2325ins12)¹ | |
| A775_G776insAVMA (c. | Lung Adenocarcinoma |
| 2324_2325ins12)¹⁴ | |
| A775_G776insSVMA⁹ | Non-Small Cell Lung Cancer⁹ |
| A775_G776insVAG¹⁴ | Lung Adenocarcinoma |
| A775insV G776C⁸ | |
| A775_G776insI⁹ | Non-Small Cell Lung Cancer⁹ |
| G776delinsVC^{2,8}; | Lung Adenocarcinoma |
| G776delinsVV⁸; G776delinsLC⁹ | |
| G776C V777insC⁸; G776C | |
| V777insV⁸ | |
| V777_G778insCG², | Lung Adenocarcinoma², Non- |
| V777_G778insGSP¹⁶ | Small Cell Lung Cancer¹⁶ |
| G778_S779insCPG⁹ | Non-Small Cell Lung Cancer⁹ |
| P780_Y781insGSP^{2,8} | Lung Adenocarcinoma |

^{A}The HER2 mutations shown may be activating mutations and/or confer increased resistance of HER2 to a HER2 inhibitor and/or a multi-kinase inhibitor (MKI), e.g., as compared to a wildtype HER2.
¹Li et al. J Thorac Oncol. 2016 March; 11(3): 414-9.
²Arcila et al. Clin Cancer Res. 2012 Sep. 15; 18(18): 10.1158/1078-0432.CCR-12-0912.
³Bose et al. Cancer Discov. 2013 February; 3(2): 224-37.
⁴Hanker et al. Cancer Discov. 2017 June; 7(6): 575-585.
⁵Christgen et al. Virchows Arch. 2018 November; 473(5): 577-582.
⁶Si et al. Cancer Biomark. 2018; 23(2): 165-171.
⁷Kavuri et al. Cancer Discov. 2015 August; 5(8): 832-841.
⁸Robichaux et al. Nat Med. 2018 May; 24(5): 638-646.
⁹Kosaka et al. Cancer Res. 2017 May 15; 77(10): 2712-2721.
¹⁰Pahuja et al. Cancer Cell. 2018 Nov. 12; 34(5): 792-806.e5.
¹¹Ross et al. Cancer. 2018 Apr. 1; 124(7): 1358-1373.
¹²Gharib et al. J Cell Physiol. 2019 August; 234(8): 13137-13144.
¹³Krawczyk et al. Oncol Lett. 2013 October; 6(4): 1063-1067.
¹⁴Lai et al. Eur J Cancer. 2019 March; 109: 28-35.
¹⁵Sun et al. J Cell Mol Med. 2015 December; 19(12): 2691-2701.
¹⁶Xu et al. Thorac Cancer. 2020 March; 11(3): 679-685.

In some embodiments, the dysregulation of a HER2 gene, a HER2 kinase, or expression or activity or level of any of the same, includes a splice variation in a HER2 mRNA which results in an expressed protein that is an alternatively spliced variant of HER2 having at least one residue deleted (as compared to the wild-type HER2 kinase) resulting in a constitutive activity of a HER2 kinase domain. In some embodiments, the splice variant of HER2 is Δ16HER-3 or p95HER-2. See, e.g., Sun et al. J Cell Mol Med. 2015 December; 19(12): 2691-2701.

In some embodiments, dysregulation of an HER2 gene, an HER2 kinase, or the expression or activity or level of any of the same can be caused by a splice variation in a HER2 mRNA that results in the expression of an altered HER2 protein that has increased resistance to inhibition by an HER2 inhibitor, a tyrosine kinase inhibitor (TKI), and/or a multi-kinase inhibitor (MKI), e.g., as compared to a wild type HER2 kinase (e.g., the HER2 variants described herein). See, e.g., Rexer and Arteaga. Crit Rev Oncog. 2012; 17(1): 1-16.

In some embodiments, the dysregulation of a HER2 gene, a HER2 kinase, or expression or activity or level of any of the same, includes one or more chromosome translocations or inversions resulting in HER2 gene fusions, respectively. In some embodiments, the dysregulation of a HER2 gene, a HER2 kinase, or expression or activity or level of any of the same, is a result of genetic translocations in which the expressed protein is a fusion protein containing residues from a non-HER2 partner protein and HER2, and include a minimum of a functional HER2 kinase domain, respectively.

TABLE 4
Exemplary HER2 Fusion Proteins and Cancers

| Non-limiting Exemplary | Non-limiting Exemplary |
|---|---|
| HER2 Fusions | HER2-associated Cancer(s) |
| ZNF207_ex2/HER2_ex18¹ | Gastric Cancer |
| MDK_ex4/HER2_ex11¹ | Gastric Cancer |
| NOS2_ex2/HER2_ex2¹ | Gastric Cancer |

¹Yu et al. J Transl Med. 2015: 13: 116.

In some embodiments, the dysregulation of a HER2 gene, a HER2 kinase, or expression or activity or level of any of the same, includes at least one point mutation in a HER2 gene that results in the production of a HER2 kinase that has one or more amino acid substitutions or insertions or deletions in a HER2 gene that results in the production of a HER2 kinase that has one or more amino acids inserted or removed, as compared to the wild-type HER2 kinase.

In other embodiments, the dysregulation of a HER2 gene, a HER2 kinase, or expression or activity or level of any of the same, includes at least one point mutation in a HER2 gene that results in the production of a HER2 kinase that has one or more amino acid substitutions as compared to the wild-type HER2 kinase, and which has increased resistance to a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, as compared to a wild type HER2 kinase or a HER2 kinase not including the same mutation.

Exemplary Sequence of Mature Human HER2 Protein
(UniProtKB entry P04626)
(SEQ ID NO: 2)
MELAALCRWG LLLALLPPGA ASTQVCTGTD MKLRLPASPE
THLDMLRHLY QGCQVVQGNL ELTYLPTNAS LSFLQDIQEV
QGYVLIAHNQ VRQVPLQRLR IVRGTQLFED NYALAVLDNG
DPLNNTTPVT GASPGGLREL QLRSLTEILK GGVLIQRNPQ
LCYQDTILWK DIFHKNNQLA LTLIDTNRSR ACHPCSPMCK
GSRCWGESSE DCQSLTRTVC AGGCARCKGP LPTDCCHEQC
AAGCTGPKHS DCLACLHFNH SGICELHCPA LVTYNTDTFE
SMPNPEGRYT FGASCVTACP YNYLSTDVGS CTLVCPLHNQ
EVTAEDGTQR CEKCSKPCAR VCYGLGMEHL REVRAVTSAN
IQEFAGCKKI FGSLAFLPES FDGDPASNTA PLQPEQLQVF
ETLEEITGYL YISAWPDSLP DLSVFQNLQV IRGRILHNGA
YSLTLQGLGI SWLGLRSLRE LGSGLALIHH NTHLCFVHTV
PWDQLFRNPH QALLHTANRP EDECVGEGLA CHQLCARGHC
WGPGPTQCVN CSQFLRGQEC VEECRVLQGL PREYVNARHC
LPCHPECQPQ NGSVTCFGPE ADQCVACAHY KDPPFCVARC
PSGVKPDLSY MPIWKFPDEE GACQPCPINC THSCVDLDDK
GCPAEQRASP LTSIISAVVG ILLVVVLGVV FGILIKRRQQ
KIRKYTMRRL LQETELVEPL TPSGAMPNQA QMRILKETEL
RKVKVLGSGA FGTVYKGIWI PDGENVKIPV AIKVLRENTS
PKANKEILDE AYVMAGVGSP YVSRLLGICL TSTVQLVTQL
MPYGCLLDHV RENRGRLGSQ DLLNWCMQIA KGMSYLEDVR
LVHRDLAARN VLVKSPNHVK ITDFGLARLL DIDETEYHAD
GGKVPIKWMA LESILRRRFT HQSDVWSYGV TVWELMTFGA
KPYDGIPARE IPDLLEKGER LPQPPICTID VYMIMVKCWM
IDSECRPRFR ELVSEFSRMA RDPQRFVVIQ NEDLGPASPL
DSTFYRSLLE DDDMGDLVDA EEYLVPQQGF FCPDPAPGAG
GMVHHRHRSS STRSGGGDLT LGLEPSEEEA PRSPLAPSEG
AGSDVFDGDL GMGAAKGLQS LPTHDPSPLQ RYSEDPTVPL
PSETDGYVAP LTCSPQPEYV NQPDVRPQPP SPREGPLPAA
RPAGATLERP KTLSPGKNGV VKDVFAFGGA VENPEYLTPQ
GGAAPQPHPP PAFSPAFDNL YYWDQDPPER GAPPSTFKGT
PTAENPEYLG LDVPV

In some embodiments, dysregulation of an HER2 gene, an HER2 kinase, or expression or activity or level of any of the same, includes at least one HER2 inhibitor resistance mutation in an HER2 gene that results in the production of an HER2 kinase that has one or more of the amino acid substitutions, insertions, or deletions as described in Table 5. In some embodiments, the compound of Formula (I) and pharmaceutically acceptable salts and solvates thereof, are useful in treating subjects that develop cancers with HER2 inhibitor resistance mutations (e.g., that result in an increased resistance to a first HER2 inhibitor, e.g., a substitution at amino acid position 755 or 798 (e.g., L755S, L755P, T798I, and T798M), and/or one or more HER2 inhibitor resistance mutations listed in Table 5) by either dosing in combination or as a subsequent or additional (e.g., follow-up) therapy to existing drug treatments (e.g., other inhibitors of HER2; e.g., first and/or second HER2 inhibitors).

TABLE 5
HER2 Protein Amino Acid Resistance Mutations

| Amino Acid | Non-Limiting | Non-limiting Exemplary |
|---|---|---|
| Position(s) | Exemplary Mutations | HER2-associated Cancer(s) |
| 726 | L726I, L726F² | Breast Cancer |
| 755 | L755S², L755P² | Breast Cancer |
| 780 | P780L² | Breast Cancer |
| 783 | S783P² | Breast Cancer |
| 785 | L785F² | Breast Cancer |
| 798 | T798I¹, T798M² | Breast Cancer |

¹Hanker et al. Cancer Discov. 2017 June; 7(6): 575-585.
²Sun et al. J Cell Mol Med. 2015 December; 19(12): 2691-2701.

As used herein, a “first inhibitor of HER2” or “first HER2 inhibitor” is a HER2 inhibitor as defined herein, but which does not include the compound of Formula (I), or a pharmaceutically acceptable salt thereof as defined herein. As used herein, a “second inhibitor of HER2” or a “second HER2 inhibitor” is a HER2 inhibitor as defined herein, but which does not include the compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, as defined herein. When both a first and a second inhibitor of HER2 are present in a method provided herein, the first and second inhibitors of HER2 are different. In some embodiments, the first and/or second inhibitor of HER2 bind in a different location than the compound of Formula (I). For example, in some embodiments, a first and/or second inhibitor of HER2 can inhibit dimerization of HER2, while the compound of Formula (I) can inhibit the active site. In some embodiments, a first and/or second inhibitor of HER2 can be an allosteric inhibitor of HER2, while the compound of Formula (I) can inhibit the HER2 active site.

Exemplary first and second inhibitors of HER2 are described herein. In some embodiments, a first or second inhibitor of HER2 can be selected from the group consisting of: trastuzumab (e.g., TRAZIMERA™, HERCEPTIN®), pertuzumab (e.g., PERJETA®), trastuzumab emtansine (T-DM1 or ado-trastuzumab emtansine, e.g., KADCYLA®), lapatinib, KU004, neratinib (e.g., NERLYNX®), dacomitinib (e.g., VIZIMPRO®), afatinib (GILOTRIF®), tucatinib (e.g., TUKYSA™), erlotinib (e.g., TARCEVA®), pyrotinib, poziotinib, CP-724714, CUDC-101, sapitinib (AZD8931), tanespimycin (17-AAG), IPI-504, PF299, pelitinib, S-22261 1, and AEE-788.

In some embodiments, compounds of Formula (I), or pharmaceutically acceptable salts and solvates thereof are useful for treating a cancer that has been identified as having one or more HER2 inhibitor resistance mutations (that result in an increased resistance to a first or second inhibitor of HER2, e.g., a substitution described in Table 5 including substitutions at amino acid position 755 or 798 (e.g., L755S, L755P, T798I, and T798M)). In some embodiments, the one or more HER2 inhibitor resistance mutations occurs in a nucleic acid sequence encoding a mutant HER2 protein (e.g., a mutant HER2 protein having any of the mutations described in Table 3) resulting in a mutant HER2 protein that exhibits HER2 inhibitor resistance.

Like EGFR, the epidermal growth factor receptor 2 (HER2) belongs to the ErbB family of receptor tyrosine kinases (RTKs) and provides critical functions in epithelial cell physiology (Schlessinger J (2014) Cold Spring Harb Perspect Biol 6, a008912; and Moasser. Oncogene. 2007 Oct. 4; 26(45): 6469-6487). It is frequently mutated and/or overexpressed in different types of human cancers and is the target of multiple cancer therapies currently adopted in the clinical practice (Moasser. Oncogene. 2007 Oct. 4; 26(45): 6469-6487).

Accordingly, provided herein are methods for treating a subject identified or diagnosed as having a HER2-associated cancer that include administering to the subject a therapeutically effective amount of a compound of Formula (I), or a pharmaceutically acceptable salt thereof, or a pharmaceutical composition thereof. In some embodiments, the subject that has been identified or diagnosed as having a HER2-associated cancer through the use of a regulatory agency-approved, e.g., FDA-approved test or assay for identifying dysregulation of a HER2 gene, a HER2 kinase, or expression or activity or level of any of the same, in a subject or a biopsy sample from the subject or by performing any of the non-limiting examples of assays described herein. In some embodiments, the test or assay is provided as a kit. In some embodiments, the cancer is a HER2-associated cancer. Also provided are methods for treating cancer in a subject in need thereof, the method comprising: (a) detecting a HER2-associated cancer in the subject; and (b) administering to the subject a therapeutically effective amount of a compound of Formula (I), or a pharmaceutically acceptable salt thereof, or a pharmaceutical composition thereof. Some embodiments of these methods further include administering to the subject another anticancer agent (e.g., a second HER2 inhibitor, or a pharmaceutically acceptable salt thereof, or an immunotherapy). In some embodiments, the subject was previously treated with a first HER2 inhibitor or previously treated with another anticancer treatment, e.g., at least partial resection of the tumor or radiation therapy. In some embodiments, the subject is determined to have a HER2-associated cancer through the use of a regulatory agency-approved, e.g., FDA-approved test or assay for identifying dysregulation of a HER2 gene, a HER2 kinase, or expression or activity or level of any of the same, in a subject or a biopsy sample from the subject or by performing any of the non-limiting examples of assays described herein. In some embodiments, the test or assay is provided as a kit. In some embodiments, the cancer is a HER2-associated cancer.

Also provided are methods of treating a subject that include performing an assay on a sample obtained from the subject to determine whether the subject has a dysregulation of a HER2 gene, a HER2 kinase, or expression or activity or level of any of the same, and administering (e.g., specifically or selectively administering) a therapeutically effective amount of a compound of Formula (I), or a pharmaceutically acceptable salt thereof, or a pharmaceutical composition thereof, to the subject determined to have a dysregulation of a HER2 gene, a HER2 kinase, or expression or activity or level of any of the same. Some embodiments of these methods further include administering to the subject another anticancer agent (e.g., a second HER2 inhibitor, or a pharmaceutically acceptable salt thereof, or immunotherapy). In some embodiments of these methods, the subject was previously treated with a first HER2 inhibitor or previously treated with another anticancer treatment, e.g., at least partial resection of a tumor or radiation therapy. In some embodiments, the subject is a subject suspected of having a HER2-associated cancer, a subject presenting with one or more symptoms of a HER2-associated cancer, or a subject having an elevated risk of developing a HER2-associated cancer. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art.

As used herein, a “first inhibitor of HER2” or “first HER2 inhibitor” is a HER2 inhibitor as defined herein, which does not include the compound of Formula (I), or a pharmaceutically acceptable salt thereof, as defined herein. As used herein, a “second inhibitor of HER2” or a “second HER2 inhibitor” is an inhibitor of HER2 as defined herein, which does not include the compound of Formula (I), or a pharmaceutically acceptable salt thereof as defined herein. When both a first and a second HER2 inhibitor are present in a method provided herein, the first and second HER2 inhibitors are different.

Also provided is the compound of Formula (I), or a pharmaceutically acceptable salt thereof or a pharmaceutical composition thereof, for use in treating a HER2-associated cancer in a subject identified or diagnosed as having a HER2-associated cancer through a step of performing an assay (e.g., an in vitro assay) on a sample obtained from the subject to determine whether the subject has a dysregulation of a HER2 gene, a HER2 kinase, or expression or activity or level of any of the same, where the presence of a dysregulation of a HER2 gene, a HER2 kinase, or expression or activity or level of any of the same, identifies that the subject has a HER2-associated cancer. Also provided is the use of a compound of Formula (I), or a pharmaceutically acceptable salt thereof for the manufacture of a medicament for treating a HER2-associated cancer in a subject identified or diagnosed as having a HER2-associated cancer through a step of performing an assay on a sample obtained from the subject to determine whether the subject has a dysregulation of a HER2 gene, a HER2 kinase, or expression or activity or level of any of the same where the presence of dysregulation of a HER2 gene, a HER2 kinase, or expression or activity or level of any of the same, identifies that the subject has a HER2-associated cancer. Some embodiments of any of the methods or uses described herein further include recording in the subject's clinical record (e.g., a computer readable medium) that the subject is determined to have a dysregulation of a HER2 gene, a HER2 kinase, or expression or activity or level of any of the same, through the performance of the assay, should be administered the compound of Formula (I), or a pharmaceutically acceptable salt thereof, or a pharmaceutical composition thereof. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy.

Also provided is the compound of Formula (I), or a pharmaceutically acceptable salt thereof, for use in the treatment of a cancer in a subject in need thereof, or a subject identified or diagnosed as having a HER2-associated cancer. Also provided is the use of the compound of Formula (I), or a pharmaceutically acceptable salt thereof, for the manufacture of a medicament for treating a cancer in a subject identified or diagnosed as having a HER2-associated cancer. In some embodiments, a subject is identified or diagnosed as having a HER2-associated cancer through the use of a regulatory agency-approved, e.g., FDA-approved, kit for identifying dysregulation of a HER2 gene, a HER2 kinase, or expression or activity or level of any of the same, in a subject or a biopsy sample from the subject. As provided herein, a HER2-associated cancer includes those described herein and known in the art.

In some embodiments of any of the methods or uses described herein, the subject has been identified or diagnosed as having a cancer with a dysregulation of a HER2 gene, a HER2 kinase, or expression or activity or level of any of the same. In some embodiments of any of the methods or uses described herein, the subject has a tumor that is positive for a dysregulation of a HER2 gene, a HER2 kinase, or expression or activity or level of any of the same. In some embodiments of any of the methods or uses described herein, the subject can be a subject with a tumor(s) that is positive for a dysregulation of a HER2 gene, a HER2 kinase, or expression or activity or level of any of the same. In some embodiments of any of the methods or uses described herein, the subject can be a subject whose tumors have a dysregulation of a HER2 gene, a HER2 kinase, or expression or activity or level of any of the same. In some embodiments of any of the methods or uses described herein, the subject is suspected of having a HER2-associated cancer. In some embodiments, provided herein are methods for treating a HER2-associated cancer in a subject in need of such treatment, the method comprising a) detecting a dysregulation of a HER2 gene, a HER2 kinase, or the expression or activity or level of any of the same in a sample from the subject; and b) administering a therapeutically effective amount of the compound of Formula (I), or a pharmaceutically acceptable salt thereof. In some embodiments, the dysregulation of a HER2 gene, a HER2 kinase, or the expression or activity or level of any of the same includes one or more HER2 kinase protein point mutations/insertions/deletions. Non-limiting examples of HER2 kinase protein fusions and point mutations/insertions/deletions are described in Tables 3-5. In some embodiments, the HER2 kinase protein point mutations/insertions/deletions are selected from the group consisting of a point mutation at amino acid position 310, 678, 755, 767, 773, 777, or 842 (e.g., S310F, S310Y, R678Q, R678W, R678P, I767M, V773M, V777L, and V842I) and/or an insertion or deletion at amino acid positions 772, 775, 776, 777, and 780 (e.g., Y772_A775dup, A775_G776insYVMA, G776delinsVC, G776delinsVV, V777_G778insGSP, and P780_Y781insGSP). In some embodiments, the HER2 kinase protein point mutation/insertion/deletion is an exon 20 point mutation/insertion/deletion. In some embodiments, the HER2 exon 20 point mutation/insertion/deletion is a point mutation at amino acid position 773, 776, 777, 779, 780, and 783 (e.g., V773M, G776C, G776V, G776S, V777L, V777M, S779T, P780L, and S783P) and/or an exon 20 insertion/deletion such as an insertion/deletion at amino acid positions 774, 775, 776, 777, 778, and 780. In some embodiments, the HER2 kinase protein insertion is an exon 20 insertion selected from the group consisting of: A775_G776insYVMA, A775_G776insAVMA, A775 G776insSVMA, A775 G776insVAG, A775insV G776C, A775_G776insI, G776del insVC2, G776del insVV, G776del insLC, G776C V777insC, G776C V777insV, V777_G778insCG, G778_S779insCPG, and P780_Y781insGSP. In some embodiments, the HER2 kinase protein mutation/insertion/deletion is an exon 20 insertion/deletion selected from the group consisting of: is Y772_A775dup, A775_G776insYVMA, G776delinsVC, G776delinsVV, V777_G778insGSP, or P780_Y781insGSP. In some embodiments, the cancer with a dysregulation of a HER2 gene, a HER2 kinase, or expression or activity or level of any of the same is determined using a regulatory agency-approved, e.g., FDA-approved, assay or kit. In some embodiments, the tumor that is positive for a dysregulation of a HER2 gene, a HER2 kinase, or expression or activity or level of any of the same is a tumor positive for one or more HER2 inhibitor resistance mutations. In some embodiments, the tumor with a dysregulation of a HER2 gene, a HER2 kinase, or expression or activity or level of any of the same is determined using a regulatory agency-approved, e.g., FDA-approved, assay or kit.

In some embodiments of any of the methods or uses described herein, the subject has a clinical record indicating that the subject has a tumor that has a dysregulation of a HER2 gene, a HER2 kinase, or expression or activity or level of any of the same. Also provided are methods of treating a subject that include administering a therapeutically effective amount of the compound of Formula (I), or a pharmaceutically acceptable salt thereof to a subject having a clinical record that indicates that the subject has a dysregulation of a HER2 gene, a HER2 kinase, or expression or activity or level of any of the same.

In some embodiments, the methods provided herein include performing an assay on a sample obtained from the subject to determine whether the subject has a dysregulation of a HER2 gene, a HER2 kinase, or expression or level of any of the same. In some such embodiments, the method also includes administering to a subject determined to have a dysregulation of a HER2 gene, a HER2 kinase, or expression or activity, or level of any of the same a therapeutically effective amount of the compound of Formula (I), or a pharmaceutically acceptable salt thereof. In some embodiments, the method includes determining that a subject has a dysregulation of a HER2 gene, a HER2 kinase, or expression or level of any of the same via an assay performed on a sample obtained from the subject. In such embodiments, the method also includes administering to a subject a therapeutically effective amount of the compound of Formula (I), or a pharmaceutically acceptable salt thereof. In some embodiments, the dysregulation in a HER2 gene, a HER2 kinase, or expression or activity or level of any of the same is one or more point mutation in the HER2 gene (e.g., any of the one or more of the HER2 point mutations described herein). The one or more point mutations in a HER2 gene can result, e.g., in the translation of a HER2 protein having one or more of the following amino acid substitutions: S310F, S310Y, R678Q, R678W, R678P, I767M, V773M, V777L, and V842I. The one or more point mutations in a HER2 gene can result, e.g., in the translation of a HER2 protein having one or more of the following exon 20 amino acid substitutions: V773M, G776C, G776V, G776S, V777L, V777M, S779T, P780L, and S783P. In some embodiments, the dysregulation in a HER2 gene, a HER2 kinase, or expression or activity or level of any of the same is one or more insertions in the HER2 gene (e.g., any of the one or more of the HER2 insertions described herein). The one or more insertions in a HER2 gene can result, e.g., in the translation of a HER2 protein having one or more of the following exon 20 insertions: M774AYVM, M774del insWLV, A775_G776insYVMA, A775_G776insAVMA, A775_G776insSVMA, A775_G776insVAG, A775insV G776C, A775_G776insI, G776del insVC2, G776del insVV, G776del insLC, G776C V777insC, G776C V777insV, V777_G778insCG, G778_S779insCPG, and P780 Y781insGSP. In some embodiments, the one or more insertions in a HER2 gene can result, e.g., in the translation of a HER2 protein having one or more of the following exon 20 insertions: Y772_A775dup, A775_G776insYVMA, G776delinsVC, G776delinsVV, V777_G778insGSP, and P780_Y781insGSP. Some embodiments of these methods further include administering to the subject another anticancer agent (e.g., a second HER2 inhibitor, or a pharmaceutically acceptable salt thereof, or immunotherapy).

In some embodiments of any of the methods or uses described herein, an assay used to determine whether the subject has a dysregulation of a HER2 gene, a HER2 kinase, or expression or activity or level of any of the same, using a sample from a subject can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labelled nucleic acid probe or at least one labelled antibody or antigen-binding fragment thereof. Assays can utilize other detection methods known in the art for detecting dysregulation of a HER2 gene, a HER2 kinase, or expression or activity or levels of any of the same (see, e.g., the references cited herein). In some embodiments, the sample is a biological sample or a biopsy sample (e.g., a paraffin-embedded biopsy sample) from the subject. In some embodiments, the subject is a subject suspected of having a HER2-associated cancer, a subject having one or more symptoms of a HER2-associated cancer, and/or a subject that has an increased risk of developing a HER2-associated cancer.

In some embodiments, dysregulation of a HER2 gene, a HER2 kinase, or the expression or activity or level of any of the same can be identified using a liquid biopsy (variously referred to as a fluid biopsy or fluid phase biopsy). See, e.g., Karachialiou et al., “Real-time liquid biopsies become a reality in cancer treatment”, Ann. Transl. Med., 3(3):36, 2016. Liquid biopsy methods can be used to detect total tumor burden and/or the dysregulation of a HER2 gene, a HER2 kinasev, or the expression or activity or level of any of the same. Liquid biopsies can be performed on biological samples obtained relatively easily from a subject (e.g., via a simple blood draw) and are generally less invasive than traditional methods used to detect tumor burden and/or dysregulation of a HER2 gene, a HER2 kinase, or the expression or activity or level of any of the same. In some embodiments, liquid biopsies can be used to detect the presence of dysregulation of a HER2 gene, a HER2 kinase, or the expression or activity or level of any of the same at an earlier stage than traditional methods. In some embodiments, the biological sample to be used in a liquid biopsy can include, blood, plasma, urine, cerebrospinal fluid, saliva, sputum, broncho-alveolar lavage, bile, lymphatic fluid, cyst fluid, stool, ascites, and combinations thereof. In some embodiments, a liquid biopsy can be used to detect circulating tumor cells (CTCs). In some embodiments, a liquid biopsy can be used to detect cell-free DNA. In some embodiments, cell-free DNA detected using a liquid biopsy is circulating tumor DNA (ctDNA) that is derived from tumor cells. Analysis of ctDNA (e.g., using sensitive detection techniques such as, without limitation, next-generation sequencing (NGS), traditional PCR, digital PCR, or microarray analysis) can be used to identify dysregulation of a HER2 gene, a HER2 kinase, or the expression or activity or level of any of the same.

Also provided is a method for inhibiting EGFR activity in a cell, comprising contacting the cell with the compound of Formula (I), or a pharmaceutically acceptable salt thereof. Also provided is a method for inhibiting HER2 activity in a cell, comprising contacting the cell with the compound of Formula (I), or a pharmaceutically acceptable salt thereof. Further provided herein is a method for inhibiting EGFR and HER2 activity in a cell, comprising contacting the cell with the compound of Formula (I), or a pharmaceutically acceptable salt thereof. In some embodiments, the contacting is in vitro. In some embodiments, the contacting is in vivo. In some embodiments, the contacting is in vivo, wherein the method comprises administering an effective amount of the compound of Formula (I), or a pharmaceutically acceptable salt thereof, to a subject having a cell having aberrant EGFR activity and/or HER2 activity. In some embodiments, the cell is a cancer cell. In some embodiments, the cancer cell is any cancer as described herein. In some embodiments, the cancer cell is an EGFR-associated cancer cell. In some embodiments, the cancer cell is a HER2-associated cancer cell. As used herein, the term “contacting” refers to the bringing together of indicated moieties in an in vitro system or an in vivo system. For example, “contacting” an EGFR kinase with a compound provided herein includes the administration of a compound provided herein to an individual or subject, such as a human, having an EGFR kinase, as well as, for example, introducing a compound provided herein into a sample containing a cellular or purified preparation containing the EGFR kinase.

Also provided herein is a method of inhibiting cell proliferation, in vitro or in vivo, the method comprising contacting a cell with an effective amount of the compound of Formula (I), or a pharmaceutically acceptable salt thereof, or a pharmaceutical composition thereof as defined herein.

Further provided herein is a method of increase cell death, in vitro or in vivo, the method comprising contacting a cell with an effective amount of the compound of Formula (I)), or a pharmaceutically acceptable salt thereof, or a pharmaceutical composition thereof as defined herein. Also provided herein is a method of increasing tumor cell death in a subject. The method comprises administering to the subject an effective compound of the compound of Formula (I), or a pharmaceutically acceptable salt thereof, in an amount effective to increase tumor cell death.

The phrase “therapeutically effective amount” means an amount of compound that, when administered to a subject in need of such treatment, is sufficient to (i) treat an EGFR kinase-associated disease or disorder or a HER2 kinase-associated disease or disorder, (ii) attenuate, ameliorate, or eliminate one or more symptoms of the particular disease, condition, or disorder, or (iii) delay the onset of one or more symptoms of the particular disease, condition, or disorder described herein. The amount of the compound of Formula (I), or a pharmaceutically acceptable salt thereof. that will correspond to such an amount will vary depending upon factors such as the particular compound, disease condition and its severity, the identity (e.g., weight) of the subject in need of treatment, but can nevertheless be routinely determined by one skilled in the art.

When employed as pharmaceuticals, the compound of Formula (I), including pharmaceutically acceptable salts or solvates thereof, can be administered in the form of pharmaceutical compositions as described herein.

Also provided herein is a method of treating a subject having a cancer, wherein the method comprises:
(a) determining that a cancer cell in a sample obtained from a subject having a cancer and previously administered one or more doses of a first EGFR inhibitor has one or more EGFR inhibitor resistance mutations that confer increased resistance to a cancer cell or tumor to treatment with the first EGFR inhibitor that was previously administered to the subject; and
(b) administering a therapeutically effective amount of the compound of Formula (I), or a pharmaceutically acceptable salt thereof, as a monotherapy or in conjunction with another anticancer agent to the subject.

Further provided herein is a method of treating a subject having a cancer, wherein the method comprises:
(a) determining that a cancer cell in a sample obtained from a subject having a cancer and previously administered one or more doses of a first EGFR inhibitor does not have one or more EGFR inhibitor resistance mutations that confer increased resistance to a cancer cell or tumor to treatment with the first EGFR inhibitor that was previously administered to the subject; and
(b) administering additional doses of the first EGFR inhibitor to the subject.

### Combinations

In the field of medical oncology it is normal practice to use a combination of different forms of treatment to treat each subject with cancer. In medical oncology the other component(s) of such conjoint treatment or therapy in addition to compositions provided herein may be, for example, surgery, radiotherapy, and chemotherapeutic agents, such as other kinase inhibitors, signal transduction inhibitors and/or monoclonal antibodies. For example, a surgery may be open surgery or minimally invasive surgery. The compound of Formula (I), or pharmaceutically acceptable salts or solvates thereof, therefore may also be useful as adjuvants to cancer treatment, that is, they can be used in combination with one or more additional therapies or therapeutic agents, for example, a chemotherapeutic agent that works by the same or by a different mechanism of action. In some embodiments, the compound of Formula (I), or a pharmaceutically acceptable salt thereof, can be used prior to administration of an additional therapeutic agent or additional therapy. For example, a subject in need thereof can be administered one or more doses of the compound of Formula (I), or a pharmaceutically acceptable salt thereof, for a period of time and then undergo at least partial resection of the tumor. In some embodiments, the treatment with one or more doses of the compound of Formula (I), or a pharmaceutically acceptable salt thereof reduces the size of the tumor (e.g., the tumor burden) prior to the at least partial resection of the tumor. In some embodiments, a subject in need thereof can be administered one or more doses of the compound of Formula (I), or a pharmaceutically acceptable salt thereof for a period of time and under one or more rounds of radiation therapy. In some embodiments, the treatment with one or more doses of the compound of Formula (I), or a pharmaceutically acceptable salt thereof, reduces the size of the tumor (e.g., the tumor burden) prior to the one or more rounds of radiation therapy.

In some embodiments, a subject has a cancer (e.g., a locally advanced or metastatic tumor) that is refractory or intolerant to standard therapy (e.g., administration of a chemotherapeutic agent, such as a first EGFR inhibitor, a first HER2 inhibitor, or a multi-kinase inhibitor, immunotherapy, or radiation (e.g., radioactive iodine)). In some embodiments, a subject has a cancer (e.g., a locally advanced or metastatic tumor) that is refractory or intolerant to prior therapy (e.g., administration of a chemotherapeutic agent, such as a first EGFR inhibitor, a first HER2 inhibitor, or a multi-kinase inhibitor, immunotherapy, or radiation (e.g., radioactive iodine)). In some embodiments, a subject has a cancer (e.g., a locally advanced or metastatic tumor) that has no standard therapy. In some embodiments, a subject is EGFR inhibitor naïve. For example, the subject is naïve to treatment with a selective EGFR inhibitor. In some embodiments, a subject is not EGFR inhibitor naïve. In some embodiments, a subject is HER2 inhibitor naïve. For example, the subject is naïve to treatment with a selective HER2 inhibitor. In some embodiments, a subject is not HER2 inhibitor naïve. In some embodiments, a subject has undergone prior therapy. For example, treatment with a multi-kinase inhibitor (MKI), an EGFR tyrosine kinase inhibitor (TKI), osimertinib, gefitinib, erlotinib, afatinib, lapatinib, neratinib, AZD-9291, CL-387785, CO-1686, or WZ4002.

In some embodiments of any the methods described herein, the compound of Formula (I) (or a pharmaceutically acceptable salt thereof) is administered in combination with a therapeutically effective amount of at least one additional therapeutic agent selected from one or more additional therapies or therapeutic (e.g., chemotherapeutic) agents.

Non-limiting examples of additional therapeutic agents include: other EGFR-targeted therapeutic agents (i.e., a first or second EGFR inhibitor), other HER2-targeted therapeutic agents (i.e., a first or second HER2 inhibitor), RAS pathway targeted therapeutic agents, PARP inhibitors, other kinase inhibitors (e.g., receptor tyrosine kinase-targeted therapeutic agents (e.g., Trk inhibitors or multi-kinase inhibitors)), farnesyl transferase inhibitors, signal transduction pathway inhibitors, checkpoint inhibitors, modulators of the apoptosis pathway (e.g., obataclax); cytotoxic chemotherapeutics, angiogenesis-targeted therapies, immune-targeted agents, including immunotherapy, and radiotherapy.

In some embodiments, the other EGFR-targeted therapeutic is a multi-kinase inhibitor exhibiting EGFR inhibition activity. In some embodiments, the other EGFR-targeted therapeutic inhibitor is selective for an EGFR kinase.

Non-limiting examples of EGFR-targeted therapeutic agents (e.g., a first EGFR inhibitor or a second EGFR inhibitor) include an EGFR-selective inhibitor, a panHER inhibitor, and an anti-EGFR antibody. In some embodiments, the EGFR inhibitor is a covalent inhibitor. In some embodiments, the EGFR-targeted therapeutic agent is osimertinib (AZD9291, merelectinib, TAGRISSO™), erlotinib (TARCEVA®), gefitinib (IRESSA®), cetuximab (ERBITUX®), necitumumab (PORTRAZZA™, IMC-11F8), neratinib (HKI-272, NERLYNX®), lapatinib (TYKERB®), panitumumab (ABX-EGF, VECTIBIX®), vandetanib (CAPRELSA®), rociletinib (CO-1686), olmutinib (OLITA™, HM61713, BI-1482694), naquotinib (ASP8273), nazartinib (EGF816, NVS-816), PF-06747775, icotinib (BPI-2009H), afatinib (BIBW 2992, GILOTRIF®), dacomitinib (PF-00299804, PF-804, PF-299, PF-299804), avitinib (AC0010), ACOO10MA EA1045, matuzumab (EMD-7200), nimotuzumab (h-R3, BIOMAb EGFR®), zalutumab, MDX447, depatuxizumab (humanized mAb 806, ABT-806), depatuxizumab mafodotin (ABT-414), ABT-806, mAb 806, canertinib (CI-1033), shikonin, shikonin derivatives (e.g., deoxyshikonin, isobutyrylshikonin, acetylshikonin, β,β-dimethylacrylshikonin and acetylalkannin), poziotinib (NOV120101, HM781-36B), AV-412, ibrutinib, WZ4002, brigatinib (AP26113, ALUNBRIG®), pelitinib (EKB-569), tarloxotinib (TH-4000, PR610), BPI-15086, Hemay022, ZN-e4, tesevatinib (KD019, XL647), YH25448, epitinib (HMPL-813), CK-101, MM-151, AZD3759, ZD6474, PF-06459988, varlintinib (ASLAN001, ARRY-334543), AP32788, HLX07, D-0316, AEE788, HS-10296, avitinib, GW572016, pyrotinib (SHR1258), SCT200, CPGJ602, Sym004, MAb-425, Modotuximab (TAB-H49), futuximab (992 DS), zalutumumab, KL-140, R05083945, IMGN289, JNJ-61186372, LY3164530, Sym013, AMG 595, BDTX-189, avatinib, Disruptin, CL-387785, EGFRBi-Armed Autologous T Cells, and EGFR CAR-T Therapy. In some embodiments, the EGFR-targeted therapeutic agent is selected from osimertinib, gefitinib, erlotinib, afatinib, lapatinib, neratinib, AZD-9291, CL-387785, CO-1686, or WZ4002.

Additional EGFR-targeted therapeutic agents (e.g., a first EGFR inhibitor or a second EGFR inhibitor) include those disclosed in WO 2019/246541; WO 2019/165385; WO 2014/176475; and U.S. Pat. No. 9,029,502, each of which is incorporated by reference in its entirety.

In some embodiments, the other HER2-targeted therapeutic is a multi-kinase inhibitor exhibiting HER2 inhibition activity. In some embodiments, the other HER2-targeted therapeutic inhibitor is selective for a HER2 kinase.

Non-limiting examples of HER2-targeted therapeutic agents (e.g., a first HER2 inhibitor or a second HER2 inhibitor) include a HER2-selective inhibitor, a panHER inhibitor, and an anti-HER2 antibody. Exemplary HER2-targeted therapeutic agents include trastuzumab (e.g., TRAZIMERA™, HERCEPTIN®), pertuzumab (e.g., PERJETA®), trastuzumab emtansine (T-DM1 or ado-trastuzumab emtansine, e.g., KADCYLA®), lapatinib, KU004, neratinib (e.g., NERLYNX®), dacomitinib (e.g., VIZIMIPRO®), afatinib (GILOTRIF®), tucatinib (e.g., TUKYSA™), erlotinib (e.g., TARCEVA®), pyrotinib, poziotinib, CP-724714, CUDC-101, sapitinib (AZD8931), tanespimycin (17-AAG), IPI-504, PF299, pelitinib, S-22261 1, and AEE-788.

Additional HER2-targeted therapeutic agents (e.g., a first HER2 inhibitor or a second HER2 inhibitor) include those disclosed in WO 2019/246541; WO 2019/165385; WO 2014/176475; and U.S. Pat. No. 9,029,502, each of which is incorporated by reference in its entirety.

A “RAS pathway targeted therapeutic agent” as used herein includes any compound exhibiting inactivation activity of any protein in a RAS pathway (e.g., kinase inhibition, allosteric inhibition, inhibition of dimerization, and induction of degradation). Non-limiting examples of a protein in a RAS pathway include any one of the proteins in the RAS-RAF-MAPK pathway or PI3K/AKT pathway such as RAS (e.g., KRAS, HRAS, and NRAS), RAF, BRAF, MEK, ERK, PI3K, AKT, and mTOR. In some embodiments, a RAS pathway modulator can be selective for a protein in a RAS pathway, e.g., the RAS pathway modulator can be selective for RAS (also referred to as a RAS modulator). In some embodiments, a RAS modulator is a covalent inhibitor. In some embodiments, a RAS pathway targeted therapeutic agent is a “KRAS pathway modulator.” A KRAS pathway modulator includes any compound exhibiting inactivation activity of any protein in a KRAS pathway (e.g., kinase inhibition, allosteric inhibition, inhibition of dimerization, and induction of degradation). Non-limiting examples of a protein in a KRAS pathway include any one of the proteins in the KRAS-RAF-MAPK pathway or PI3K/AKT pathway such as KRAS, RAF, BRAF, MEK, ERK, PI3K, AKT, and mTOR. In some embodiments, a KRAS pathway modulator can be selective for a protein in a RAS pathway, e.g., the KRAS pathway modulator can be selective for KRAS (also referred to as a KRAS modulator). In some embodiments, a KRAS modulator is a covalent inhibitor. Non-limiting examples of a KRAS-targeted therapeutic agents (e.g., KRAS inhibitors) include BI 1701963, AMG 510, ARS-3248, ARS1620, AZD4785, SML-8-73-1, SML-10-70-1, VSA9, AA12, and MRTX-849.

Further non-limiting examples of RAS-targeted therapeutic agents include BRAF inhibitors, MEK inhibitors, ERK inhibitors, PI3K inhibitors, AKT inhibitors, and mTOR inhibitors. In some embodiments, the BRAF inhibitor is vemurafenib (ZELBORAF®), dabrafenib (TAFINLAR®), and encorafenib (BRAFTOVI™), BMS-908662 (XL281), sorafenib, LGX818, PLX3603, RAF265, R05185426, GSK2118436, ARQ 736, GDC-0879, PLX-4720, AZ304, PLX-8394, HM95573, RO5126766, LXH254, or a combination thereof.

In some embodiments, the MEK inhibitor is trametinib (MEKINIST®, GSK1120212), cobimetinib (COTELLIC®), binimetinib (MEKTOVI®, MEK162), selumetinib (AZD6244), PD0325901, MSC1936369B, SHR7390, TAK-733, RO5126766, CS3006, WX-554, PD98059, CI1040 (PD184352), hypothemycin, or a combination thereof.

In some embodiments, the ERK inhibitor is FRI-20 (ON-01060), VTX-Ile, 25-OH-D3-3-BE (B3CD, bromoacetoxycalcidiol), FR-180204, AEZ-131 (AEZS-131), AEZS-136, AZ-13767370, BL-EI-001, LY-3214996, LTT-462, KO-947, KO-947, MK-8353 (SCH900353), SCH772984, ulixertinib (BVD-523), CC-90003, GDC-0994 (RG-7482), ASN007, FR148083, 5-7-Oxozeaenol, 5-iodotubercidin, GDC0994, ONC201, or a combination thereof.

In some embodiments, PI3K inhibitor is selected from buparlisib (BKM120), alpelisib (BYL719), WX-037, copanlisib (ALIQOPA™, BAY80-6946), dactolisib (NVP-BEZ235, BEZ-235), taselisib (GDC-0032, RG7604), sonolisib (PX-866), CUDC-907, PQR309, ZSTK474, SF1126, AZD8835, GDC-0077, ASN003, pictilisib (GDC-0941), pilaralisib (XL147, SAR245408), gedatolisib (PF-05212384, PKI-587), serabelisib (TAK-117, MLN1117, INK 1117), BGT-226 (NVP-BGT226), PF-04691502, apitolisib (GDC-0980), omipalisib (GSK2126458, GSK458), voxtalisib (XL756, SAR245409), AMG 511, CH5132799, GSK1059615, GDC-0084 (RG7666), VS-5584 (SB2343), PKI-402, wortmannin, LY294002, PI-103, rigosertib, XL-765, LY2023414, SAR260301, KIN-193 (AZD-6428), GS-9820, AMG319, GSK2636771, or a combination thereof.

In some embodiments, the AKT inhibitor is selected from miltefosine (IMPADIVO®), wortmannin, NL-71-101, H-89, GSK690693, CCT128930, AZD5363, ipatasertib (GDC-0068, RG7440), A-674563, A-443654, AT7867, AT13148, uprosertib, afuresertib, DC120, 2-[4-(2-aminoprop-2-yl)phenyl]-3-phenylquinoxaline, MK-2206, edelfosine, miltefosine, perifosine, erucylphophocholine, erufosine, SR13668, OSU-A9, PH-316, PHT-427, PIT-1, DM-PIT-1, triciribine (Triciribine Phosphate Monohydrate), API-1, N-(4-(5-(3-acetamidophenyl)-2-(2-aminopyridin-3-yl)-3H-imidazo[4,5-b]pyridin-3-yl)benzyl)-3-fluorobenzamide, ARQ092, BAY 1125976, 3-oxo-tirucallic acid, lactoquinomycin, boc-Phe-vinyl ketone, Perifosine (D-21266), TCN, TCN-P, GSK2141795, ONC201, or a combination thereof.

In some embodiments, the mTOR inhibitor is selected from MLN0128, AZD-2014, CC-223, AZD2014, CC-115, everolimus (RAD001), temsirolimus (CCI-779), ridaforolimus (AP-23573), sirolimus (rapamycin), or a combination thereof.

Non-limiting examples of farnesyl transferase inhibitors include lonafarnib, tipifarnib, BMS-214662, L778123, L744832, and FTI-277.

In some embodiments, a chemotherapeutic agent includes an anthracycline, cyclophosphamide, a taxane, a platinum-based agent, mitomycin, gemcitabine, eribulin (HALAVEN™), or combinations thereof.

Non-limiting examples of a taxane include paclitaxel, docetaxel, abraxane, and taxotere.

In some embodiments, the anthracycline is selected from daunorubicin, doxorubicin, epirubicin, idarubicin, and combinations thereof.

In some embodiments, the platinum-based agent is selected from carboplatin, cisplatin, oxaliplatin, nedplatin, triplatin tetranitrate, phenanthriplatin, picoplatin, satraplatin and combinations thereof.

Non-limiting examples of PARP inhibitors include olaparib (LYNPARZA®), talazoparib, rucaparib, niraparib, veliparib, BGB-290 (pamiparib), CEP 9722, E7016, iniparib, IMP4297, NOV1401, 2X-121, ABT-767, RBN-2397, BMN 673, KU-0059436 (AZD2281), BSI-201, PF-01367338, INO-1001, and JPI-289.

Non-limiting examples of immunotherapy include immune checkpoint therapies, atezolizumab (TECENTRIQ®), albumin-bound paclitaxel. Non-limiting examples of immune checkpoint therapies include inhibitors that target CTLA-4, PD-1, PD-L1, BTLA, LAG-3, A2AR, TIM-3, B7-H3, VISTA, IDO, and combinations thereof. In some embodiments the CTLA-4 inhibitor is ipilimumab (YERVOY®). In some embodiments, the PD-1 inhibitor is selected from pembrolizumab (KEYTRUDA®), nivolumab (OPDIVO®), cemiplimab (LIBTAYO®), or combinations thereof. In some embodiments, the PD-L1 inhibitor is selected from atezolizumab (TECENTRIQ®), avelumab (BAVENCIO®), durvalumab (IMFINZI®), or combinations thereof. In some embodiments, the LAG-3 inhibitor is IMP701 (LAG525). In some embodiments, the A2AR inhibitor is CPI-444. In some embodiments, the TIM-3 inhibitor is MBG453. In some embodiments, the B7-H3 inhibitor is enoblituzumab. In some embodiments, the VISTA inhibitor is JNJ-61610588. In some embodiments, the IDO inhibitor is indoximod. See, for example, Marin-Acevedo, et al., J Hematol Oncol. I1: 39 (2018).

In some embodiments, the additional therapy or therapeutic agent is a combination of atezolizumab and nab-paclitaxel.

Accordingly, also provided herein is a method of treating cancer, comprising administering to a subject in need thereof a pharmaceutical combination for treating cancer which comprises (a) a compound of Formula (I), or a pharmaceutically acceptable salt thereof, (b) an additional therapeutic agent, and (c) optionally at least one pharmaceutically acceptable carrier for simultaneous, separate or sequential use for the treatment of cancer, wherein the amounts of the compound of Formula (I), or a pharmaceutically acceptable salt thereof, and the additional therapeutic agent are together effective in treating the cancer.

In some embodiments, the additional therapeutic agent(s) includes any one of the above listed therapies or therapeutic agents which are standards of care in cancers wherein the cancer has a dysregulation of an EGFR gene, an EGFR protein, or expression or activity, or level of any of the same.

In some embodiments, the additional therapeutic agent(s) includes any one of the above listed therapies or therapeutic agents which are standards of care in cancers wherein the cancer has a dysregulation of a HER2 gene, a HER2 kinase, or expression or activity, or level of any of the same.

These additional therapeutic agents may be administered with one or more doses of the compound of Formula (I), or a pharmaceutically acceptable salt thereof, or pharmaceutical composition thereof, as part of the same or separate dosage forms, via the same or different routes of administration, and/or on the same or different administration schedules according to standard pharmaceutical practice known to one skilled in the art.

Also provided herein is (i) a pharmaceutical combination for treating a cancer in a subject in need thereof, which comprises (a) a compound of Formula (I), or a pharmaceutically acceptable salt thereof, (b) at least one additional therapeutic agent (e.g., any of the exemplary additional therapeutic agents described herein or known in the art), and (c) optionally at least one pharmaceutically acceptable carrier for simultaneous, separate or sequential use for the treatment of cancer, wherein the amounts of the compound of Formula (I), or pharmaceutically acceptable salt thereof, and of the additional therapeutic agent are together effective in treating the cancer; (ii) a pharmaceutical composition comprising such a combination; (iii) the use of such a combination for the preparation of a medicament for the treatment of cancer; and (iv) a commercial package or product comprising such a combination as a combined preparation for simultaneous, separate or sequential use; and to a method of treatment of cancer in a subject in need thereof. In some embodiments, the cancer is an EGFR-associated cancer. For example, an EGFR-associated cancer having one or more EGFR inhibitor resistance mutations. In some embodiments, the cancer is a HER2-associated cancer. For example, a HER2-associated cancer having one or more HER2 inhibitor resistance mutations.

The term “pharmaceutical combination”, as used herein, refers to a pharmaceutical therapy resulting from the mixing or combining of more than one active ingredient and includes both fixed and non-fixed combinations of the active ingredients. The term “fixed combination” means that a compound of Formula (I), or a pharmaceutically acceptable salt thereof, and at least one additional therapeutic agent (e.g., a chemotherapeutic agent), are both administered to a subject simultaneously in the form of a single composition or dosage. The term “non-fixed combination” means that a compound of Formula (I), or a pharmaceutically acceptable salt thereof, and at least one additional therapeutic agent (e.g., chemotherapeutic agent) are formulated as separate compositions or dosages such that they may be administered to a subject in need thereof simultaneously, concurrently or sequentially with variable intervening time limits, wherein such administration provides effective levels of the two or more compounds in the body of the subject. These also apply to cocktail therapies, e.g., the administration of three or more active ingredients.

Accordingly, also provided herein is a method of treating a cancer, comprising administering to a subject in need thereof a pharmaceutical combination for treating cancer which comprises (a) the compound of Formula (I), or pharmaceutically acceptable salt thereof, and (b) an additional therapeutic agent, wherein the compound of Formula (I) and the additional therapeutic agent are administered simultaneously, separately or sequentially, wherein the amounts of the compound of Formula (I), or pharmaceutically acceptable salt thereof, and the additional therapeutic agent are together effective in treating the cancer. In some embodiments, the compound of Formula (I), or a pharmaceutically acceptable salt thereof, and the additional therapeutic agent are administered simultaneously as separate dosages. In some embodiments, the compound of Formula (I), or pharmaceutically acceptable salt thereof, and the additional therapeutic agent are administered as separate dosages sequentially in any order, in jointly therapeutically effective amounts, e.g., in daily or intermittently dosages. In some embodiments, the compound of Formula (I), or pharmaceutically acceptable salt thereof, and the additional therapeutic agent are administered simultaneously as a combined dosage. In some embodiments, the cancer is an EGFR-associated cancer. For example, an EGFR-associated cancer having one or more EGFR inhibitor resistance mutations. In some embodiments, the cancer is a HER2-associated cancer. For example, a HER2-associated cancer having one or more HER2 inhibitor resistance mutations.

In some embodiments, the presence of one or more EGFR inhibitor resistance mutations in a tumor causes the tumor to be more resistant to treatment with a first EGFR inhibitor. Methods useful when an EGFR inhibitor resistance mutation causes the tumor to be more resistant to treatment with a first EGFR inhibitor are described below. For example, provided herein are methods of treating a subject having a cancer that include: identifying a subject having a cancer cell that has one or more EGFR inhibitor resistance mutations; and administering to the identified subject the compound of Formula (I), or a pharmaceutically acceptable salt thereof. In some embodiments, the compound of Formula (I), or a pharmaceutically acceptable salt thereof is administered in combination with the first EGFR inhibitor. Also provided are methods of treating a subject identified as having a cancer cell that has one or more EGFR inhibitor resistance mutations that include administering to the subject a compound of Formula (I), or a pharmaceutically acceptable salt thereof. In some embodiments, the compound of Formula (I), or a pharmaceutically acceptable salt thereof, is administered in combination with the first EGFR inhibitor. In some embodiments, the one or more EGFR inhibitor resistance mutations confer increased resistance to a cancer cell or tumor to treatment with the first EGFR inhibitor. In some embodiments, the one or more EGFR inhibitor resistance mutations include one or more EGFR inhibitor resistance mutations listed in Table 2a and Table 2b. For example, the one or more EGFR inhibitor resistance mutations can include a substitution at amino acid position 718, 747, 761, 790, 797, or 854 (e.g., L718Q, L747S, D761Y, T790M, C797S, and T854A).

For example, provided herein are methods for treating an EGFR-associated cancer in a subject in need of such treatment, the method comprising (a) detecting a dysregulation of an EGFR gene, an EGFR kinase, or the expression or activity or level of any of the same in a sample from the subject; and (b) administering to the subject a therapeutically effective amount of a first EGFR inhibitor, wherein the first EGFR inhibitor is selected from the group consisting of osimertinib, gefitinib, erlotinib, afatinib, lapatinib, neratinib, AZD-9291, CL-387785, CO-1686, or WZ4002. In some embodiments, the methods further comprise (after (b)) (c) determining whether a cancer cell in a sample obtained from the subject has at least one EGFR inhibitor resistance mutation; and (d) administering the compound of Formula (I), or a pharmaceutically acceptable salt thereof, as a monotherapy or in conjunction with another anticancer agent to the subject if the subject has been determined to have a cancer cell that has at least one EGFR inhibitor resistance mutation; or (e) administering additional doses of the first EGFR inhibitor of step (b) to the subject if the subject has not been determined to have a cancer cell that has at least one EGFR inhibitor resistance mutation.

Methods useful when a HER2 activating mutation is present in a tumor are described herein. For example, provided herein are methods of treating a subject having a cancer that include: identifying a subject having a cancer cell that has one or more HER2 activating mutations; and administering to the identified subject the compound of Formula (I), or a pharmaceutically acceptable salt thereof. Also provided are methods of treating a subject identified as having a cancer that has one or more HER2 activating mutations that include administering to the subject the compound of Formula (I), or a pharmaceutically acceptable salt thereof. In some embodiments, the one or more HER2 activating mutations include one or more HER2 activating mutations listed in Tables 3-5.

Methods useful when an activating mutation (e.g., HER2 activating mutation) is present in a tumor in a subject are described herein. For example, provided herein are methods of treating a subject having a cancer that include: identifying a subject having a cancer cell that has one or more HER2 activating mutations; and administering to the identified subject a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof.

### Compound Preparation

The compound disclosed herein can be prepared in a variety of ways using commercially available starting materials, compounds known in the literature, or from readily prepared intermediates, by employing standard synthetic methods and procedures either known to those skilled in the art, or in light of the teachings herein. The synthesis of the compound disclosed herein can be achieved by generally following the scheme below, with modification for specific desired substituents.

Standard synthetic methods and procedures for the preparation of organic molecules and functional group transformations and manipulations can be obtained from the relevant scientific literature or from standard textbooks in the field. Although not limited to any one or several sources, classic texts such as R. Larock, Comprehensive Organic Transformations, VCH Publishers (1989); L. Fieser and M. Fieser, Fieser and Fieser's Reagents for Organic Synthesis, John Wiley and Sons (1994); Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. The following descriptions of synthetic methods are designed to illustrate, but not to limit, general procedures for the preparation of compound of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

In some embodiments, the compound described herein can be generated, e.g., by oxidation of compound 362 in WO 2022/066734 or a stereoisomeric mixture thereof:

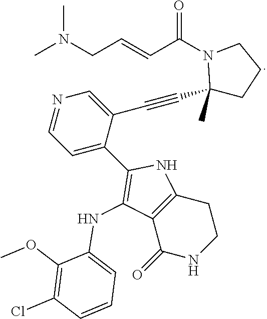 [SMILES: COc1c(Cl)cccc1Nc1c(-c2ccncc2C#C[C@@]2(C)CCCN2C(=O)/C=C/CN(C)C)[nH]c2c1C(=O)NCC2]

In certain embodiments, the compound described herein can be generated, e.g., in vivo, e.g., from compound 362 in WO 2022/066734 or a stereoisomer thereof or a stereoisomeric mixture thereof. For example, the compound described herein can be generated, in vivo in a mammal (e.g., a human) from compound 362 in WO 2022/066734 or a stereoisomer thereof or a stereoisomeric mixture thereof.

While not wishing to be bound by theory, it is believed that the in vivo transformation is facilitated by the presence of oxidative endogenous proteins (e.g., enzymes), e.g., oxidative human endogenous proteins (e.g., enzymes). A non-limiting example is cytochrome P450. In other embodiments, the compound described herein can be generated, e.g., in vitro, by hepatocyte incubation from compound 362 in WO 2022/066734 or a stereoisomer thereof or a stereoisomeric mixture thereof.

### Compound Preparation

### Synthesis of the Compound of the Compound of Formula (I):

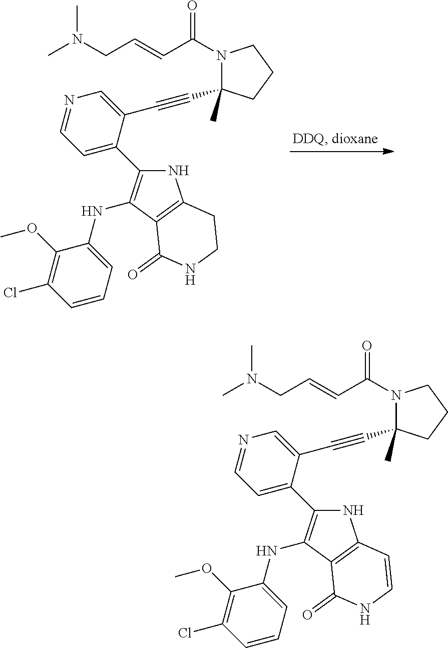 [SMILES: COc1c(Cl)cccc1Nc1c(-c2ccncc2C#C[C@@]2(C)CCCN2C(=O)/C=C/CN(C)C)[nH]c2c1C(=O)NCC2 ; COc1c(Cl)cccc1Nc1c(-c2ccncc2C#C[C@@]2(C)CCCN2C(=O)/C=C/CN(C)C)[nH]c2cc[nH]c(=O)c12]

A mixture of 3-[(3-chloro-2-methoxyphenyl)amino]-2-(3-{2-[(2R)-1-[(2E)-4-(dimethylamino)but-2-enoyl]-2-methylpyrrolidin-2-yl]ethynyl}pyridin-4-yl)-1H,5H,6H,7H-pyrrolo[3,2-c]pyridin-4-one (100 mg, 0.170 mmol, 1 equiv)1 and DDQ (57.99 mg, 0.255 mmol, 1.5 equiv) in MeCN (4 mL) was stirred for 2 h at 100° C. under nitrogen atmosphere. The resulting mixture was washed with 2×10 mL of EtOAc. The residue was purified by reverse flash chromatography with the following conditions: column, silica gel; mobile phase, MeCN in water, 10% to 50% gradient in 10 min; detector, UV 254 nm to crude product as a brown solid. The crude product was purified by reverse phase flash with the following conditions (Column: XBridge Prep OBD C18 Column, 30*150 mm, 5 m; Mobile Phase A: Water(10 mmol/L NH₄HCO₃), Mobile Phase B: ACN; Flow rate: 60 mL/min; Gradient: 30% B to 60% B in 7 min; Wave Length: 254 nm; RT1 (min): 6.5) to afford 3-[(3-chloro-2-methoxyphenyl)amino]-2-(3-{2-[(2R)-1-[(2E)-4-(dimethylamino)but-2-enoyl]-2-methylpyrrolidin-2-yl]ethynyl}pyridin-4-yl)-1H,5H-pyrrolo[3,2-c]pyridin-4-one (11.6 mg, 11.07%) as a light yellow solid. 1 This starting material can be prepared using procedures described in WO 2022/066734.

LC-MS: (M+H)⁺ found 585.10.

¹H NMR (400 MHz, Chloroform-d) δ 11.60 (s, 1H), 9.35 (s, 1H), 8.52 (s, 1H), 8.15 (s, 1H), 7.59 (d, J=5.4 Hz, 1H), 7.45 (s, 1H), 7.12-6.91 (m, 3H), 6.78-6.68 (m, 1H), 6.62 (t, J=8.1 Hz, 2H), 6.26-6.19 (m, 1H), 4.14 (s, 3H), 3.92-3.81 (m, 2H), 3.46 (s, 2H), 2.56 (s, 6H), 2.20 (s, 1H), 2.25-2.13 (m, 4H), 1.78-1.72 (m, 3H).

### Bioactivity

### Example A. EGFR Activity

Cell lines are generated by transducing Ba/F3 cells with retroviruses containing vectors with EGFR WT, EGFR exon 20 NPG Ins D770_N771, EGFR exon 20 ASV Ins V769_D770, EGFR exon 20 SVD Ins D770_N771, or EGFR exon 20 FQEA Ins A763_V764 genes and a puromycin selection marker. Transduced cells are selected with puromycin for 7 days and are then be transferred into culture media without Interleukin 3 (IL3). EGFR WT cells are maintained with supplemental EGF. Surviving cells are confirmed to express EGFR by Western blot and maintained as a pool. The IC50 data are included in Table 6.

### Study Design

### 1 Cell Seeding

1.1 Cells are harvested from flask into cell culture medium and the cell number counted.
1.2 Cells are diluted with culture medium to the desired density and 40 NL of cell suspension is added into each well of 384-well cell culture plate and the seeding density is 600 cells/well.

### 2 Compound Preparation and Treatment

2.1 Test compounds are dissolved to 10 mM in a DMSO stock solution. 45 μL of stock solution is transferred to a 384 polypropylene plate (pp-plate). Perform 3-fold, 10-point dilution via transferring 15 μL compound into 30 μL DMSO using a TECAN (EVO200) liquid handler.
2.2 Spin plates at room temperature at 1,000 RPM for 1 minute.
2.3 Transfer 120 nL of diluted compound from compound source plate into the cell plate.
2.4 After compound treatment for 72 hours, perform CTG detection for compound treatment plates as described in “Detection” section.

### 3 Detection

3.1 Plates are removed from incubators and equilibrated at room temperature for 15 minutes.
3.2 Thaw the CellTiter Glo reagents and allow to equilibrate to room temperature before the experiment.
3.3 Add 40 μL of CellTiter-Glo reagent into each well (at 1:1 to culture medium). Then place the plates at room temperature for 30 min followed by reading on EnVision.

### 4 Data Analysis

4.1 Inhibition activity is calculated following the formula below:

$$\% Inhibition = 100 \times ( LumHC - LumSample ) / ( LumHC - LumLC )$$

where HC is obtained from cells treated with 0.1% DMSO only; and LC is obtained from culture medium only.
4.2.2. Calculate the IC₅₀ by fitting the Curve using Xlfit (v5.3.1.3), equation 201:

$$Y = Bottom + ( Top - Bottom ) / ( 1 + 10 \hat{} ( ( Log {IC}_{50} - X ) * HillSlope ) )$$

The IC50 date are included in Table 6.

### Example B. Inhibitor Activity on EGFR Phosphorylation (pEGFR)

EGFR mutant Ba/F3 cells were generated by transduction with retrovirus containing vectors expressing EGFR exon 20 NPG Ins D770_N771, EGFR exon 20 ASV Ins V769_D770, or EGFR exon 20 SVD Ins D770_N771 genes along with a puromycin selection marker. Transduced cells are selected with puromycin for 7 days and are then be transferred into culture media without Interleukin 3 (IL3). Surviving cells are confirmed to express EGFR by Western blot and maintained as a pool. CUTO14 cells were obtained from Dr. Robert C. Doebele at the University of Colorado. The IC50 data are included in Table 6.

### Study Design

### 1 Cell Seeding

1.1 Cells are harvested from flask into cell culture medium and the cell number counted.
1.2 Cells are diluted with culture medium to the desired density and 40 μL of cell suspension is added into each well of 384-well cell culture plate and the seeding density is 50K cells/well (Ba/F3) or 12.5K cells/well (CUTO14).

### 2 Compound Preparation and Treatment

2.1 Test compounds are dissolved to 10 mM in a DMSO stock solution. 45 μL of stock solution is transferred to a 384 polypropylene plate (pp-plate). Perform 3-fold, 10-point dilution via transferring 15 μL compound into 30 μL DMSO using a TECAN (EV0200) liquid handler.
2.2 Spin plates at room temperature at 1,000 RPM for 1 minute.
2.3 Transfer 5 nL of diluted compound from compound source plate into the cell plate.
2.4 After compound treatment for 2 hours, perform pEGFR detection by AlphaLISA for compound treatment plates as described in “Detection” section.
   3 Detection by pEGFR AlphaLISA (Perkin-Elmer)
3.1 Plates are removed from incubators and equilibrated at room temperature for 10 minutes, and media was removed
3.2 10 μL of lysis buffer is added and plates shaken at 600 rpm for 1 hr.
3.3 Prepare acceptor mix just before use and dispense 5 μL of acceptor mix to all the wells. Shake 350 rpm for 1 hr in the dark
3.4 Prepare donor mix under low light conditions prior to use. Dispense 5 L of donor mix to all the wells. Mix well on the shaker, seal and wrap in aluminum foil and incubate 1.5 hrs at room temperature in the dark
3.5 Transfer 18.5 μL mixture to OptiPlate 384, and read using an Envision.

TABLE 6
IC50 Data for EGFR Activity and Inhibitor
Activity on EGFR phosphorylation (pEGFR)¹

| | BaF3 ASV | BaF3 SVD | BaF3 NPG | BaF3 WT | CUTO14 IC50 |
|---|---|---|---|---|---|
| Comp. | IC50 | IC50 | IC50 | IC50 | (pEGFR) |
| (I) | +++ | +++ | +++ | + | +++ |
| Compound 635 | ++ | ++ | +++ | + | +++ |
| WO | | | | | |
| 2022/066734 | | | | | |

¹“+++” indicates that IC50 < 100 nM;
“++” indicates that 100 nM <= IC50 < 1000 nM;
“+” indicates that IC50 >= 1000 nM.
“NA” indicates that the IC50 data is not available for this compound;

A number of embodiments of the invention have been described. Accordingly, other embodiments are within the scope of the following claims.

1-109. (canceled)

110. A compound having formula (I), or a pharmaceutically acceptable salt thereof:

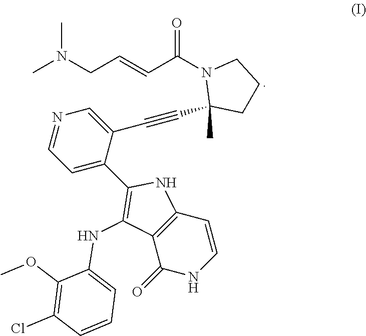 [SMILES: COc1c(Cl)cccc1Nc1c(-c2ccncc2C#C[C@@]2(C)CCCN2C(=O)/C=C/CN(C)C)[nH]c2cc[nH]c(=O)c12]

111. A pharmaceutical composition comprising a compound of claim 110, or a pharmaceutically acceptable salt thereof, and pharmaceutically acceptable diluent or carrier.

112. A method for treating cancer in a subject in need thereof, the method comprising administering to the subject a therapeutically effective amount of a compound of claim 110.

113. The method of claim 112, wherein the subject has been identified or diagnosed as having an EGFR-associated cancer.

114. The method of claim 113, wherein the EGFR-associated cancer is due to a dysregulation in an EGFR gene, an EGFR kinase protein, or expression or activity or level of any of the same is one or more point mutations in the EGFR gene.

115. The method of claim 114, wherein the one or more point mutations in an EGFR gene results in the translation of an EGFR protein having one or more amino acid substitutions at one or more of the following amino acid positions exemplified in Table 1a and Table 1 b.

116. The method of claim 114, wherein the one or more point mutations is selected from the mutations in Table 1a and Table 1b (e.g., L858R, G719S, G719C, G719A, L861Q, a deletion in exon 19 and/or an insertion in exon 20).

117. The method of claim 114, wherein the one or more point mutations is an EGFR inhibitor resistance mutation (e.g., L718Q, L747S, D761Y, T790M, C797S, T854A).

118. The method of claim 114, wherein the one or more point mutations in an EGFR gene include a deletion in exon 19 of a human EGFR gene.

119. The method of claim 114, wherein the one or more mutations is an EGFR insertion in exon 20 of a human EGFR gene.

120. The method of claim 119, wherein the insertion in exon 20 of a human EGFR gene is selected from: V769_D770insX, D770_N771 insX, N771_P772insX, P772_H773insX, and H773_V774insX.

121. The method of claim 119, wherein the insertion in exon 20 of a human EGFR gene is selected from: Y772_A775dup, A775_G776insYVMA, G776delinsVC, G776delinsVV, V777_G778insGSP, and P780_Y781 insGSP.

122. The method of claim 113, wherein the EGFR-associated cancer is selected from the group consisting of: oral cancer, oropharyngeal cancer, nasopharyngeal cancer, respiratory cancer, urogenital cancer, gastrointestinal cancer, central or peripheral nervous system tissue cancer, an endocrine or neuroendocrine cancer, a hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma Li-Fraumeni tumor, thyroid cancer, parathyroid cancer, pituitary tumors, adrenal gland tumors, osteogenic sarcoma tumors, breast cancer, lung cancer such as non-small cell lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer, and skin cancer.

123. The method of claim 113, wherein the EGFR-associated cancer is selected from the group consisting of: lung cancer such as non-small cell lung cancer, pancreatic cancer, head and neck cancer, melanoma, colon cancer, renal cancer, leukemia, glioblastoma, and breast cancer.

124. The method of claim 112, wherein the cancer is a HER2-associated cancer.

125. The method of claim 124, wherein the HER2-associated cancer is associated with a dysregulation of a HER2 gene, a HER2 kinase, or expression or activity or level of any of the same.

126. The method of claim 124, wherein determining that the cancer in the subject is a HER2-associated cancer includes performing an assay to detect dysregulation in a HER2 gene, a HER2 kinase protein, or expression or activity or level of any of the same in a sample from the subject.

127. The method of claim 125, wherein the dysregulation in a HER2 gene, a HER2 kinase protein, or expression or activity or level of any of the same is one or more point mutations in the HER2 gene.

128. The method of claim 127, wherein the one or more point mutations in a HER2 gene results in the translation of a HER2 protein having one or more amino acid substitutions at one or more of the following amino acid positions exemplified in Table 3.

129. The method of claim 128, wherein the one or more point mutations is selected from the mutations in Table 3 (e.g., S310F, S310Y, R678Q, R678W, R678P, I767M, V773M, V777L, and V842I).

130. The method of claim 124, wherein the cancer is selected from the group consisting of: non-small cell lung cancer, pancreatic cancer, and colorectal cancer.

131. The method of claim 112, further comprising administering an additional therapy or therapeutic agent to the subject, for example selected from: radiotherapy, cytotoxic chemotherapeutics, kinase targeted-therapeutics such as a tyrosine kinase inhibitor, in particular a second EGFR inhibitor, apoptosis modulators, signal transduction inhibitors, immune-targeted therapies, and angiogenesis-targeted therapies; especially osimertinib, gefitinib, erlotinib, afatinib, lapatinib, neratinib, AZD-9291, CL-387785, CO-1686, WZ4002, and combinations thereof; or a HER2 inhibitor such as trastuzumab, pertuzumab, trastuzumab emtansine, lapatinib, KU004, neratinib, dacomitinib, afatinib, tucatinib, erlotinib, pyrotinib, poziotinib, CP-724714, CUDC-101, sapitinib (AZD8931), tanespimycin (17-AAG), IPI-504, PF299, pelitinib, S-22261 1, and AEE-788.

132. A method for treating cancer in a subject in need thereof, the method comprising administering to the subject a therapeutically effective amount of a pharmaceutical composition according to claim 111.


SEQUENCE LISTING (2 sequences)

SEQ ID NO: 1 - protein, 1210 aa, Homo sapiens [sequence omitted]

SEQ ID NO: 2 - protein, 1255 aa, Homo sapiens [sequence omitted]